SEQUENCE LISTING (47 sequences)

SEQ ID NO: 1 - DNA, 13870 bp, Homo sapiens
gctctctaactcacagcgagctcgctgcccaaagtcctgctccgggggcttcctgggtggacctgaccgcgttcgggtgcacgtggggcgactcacacctgacaagtaaagcgggtgaggccgcgcctgtgaagggcgcctggctcctccgcaggacggtgcggcgcggcgcccccggctggaaccaggtgtaactgcagagaccctgggatcgcaggaacggctggcggcaggactgtccctacctcgagaaggtgacggggtttcctgcgctgccagccgatgaggcggccgtgacgcagcccgccgtgcagagtccccgtcggccgacaggcgtgcagagctctgcagaggacccttccgccctctgggcagcctgccaagccgtggcacccccaacccccagcactgggcacttgggagcattgcagccgccctggctcgtaccggtgccggtgctttgggcacctgggctggtttggacatgggtgccccgggcagagtccatttatgcaggtcagaatcagtgtgtggagcctgcatagacttgccctggagcggctgcctgtgctggggtggggaggagtagagggcgagaagttggtggggaagggaagcggcgccaaaagaatacccacaacatcttgcacctggaaggcaaagcagagggcagtgatctctgcagacttgcgggggcgacgcctgaagcaaacagggacatacaagctggtgccttctgtggttgtgcatggggtcttcatgcttcctgtctgagttcccagaagcttgtctctgcttttctaggcagctgccacagcctgtcacaaacagctcctggttctccacttctcatagtctcgatttcaaaatccattgcctcaccctccacctcctctccacctccacccctcctagcacctcctgactgcttgtgttctgtgtctccccactgtctcccaacctggggtggggttgggggggatgtctttcctcctgtctgctctttgatgtccagctgaagtgtcacctcctacaggcagcctcccctggctatgccagcttgtactgattgccctctcctctgaattctgtaagcatttcctatgtgtacctgcccctgggcaaggtgggcctgacttgttagagtgttagagttttaccctgttcctctaggagggcctggtaccaccacagcccagcatggtgtggtgcctcagcaggaggcatctggttacaatcaacacaagctgttccagccaatttaaagaaacttcaggaggaatagggttttaggagggcatggggaccctcctgcacccgaagccaggatgtgccaccaatcataaggaggcaggggcctccttccgctgctccctgggactctcyaggtgtccgtggcctcagcccccctctgcacacctgcatcttccttctcatcagcttcctctgctttaagcgtaaacatggatgcccaggacctggcctcaatcttccgagtctggtacttatggtgtactgacagtgtgagaccctactcctctgatcaatcccctgggttggtgacttccctgtgcaatcaatggaagccagcgaggcagggtcacatgccccgtttagaggtgcagacttggagaaggaacgtgggcaagtcttcccaggaacaggtagggcagggaggaaaggggggcatctctggtgcagcccggttcggagcaggaagacgcttaataaatgctgatagactgcaggacacaggcaaaggtgctgagctggaccctttatttctgcccttctcccttctggcaccccggccaggaaattgctgcagcctttctggaatcccgttcatttttcttactggtccacaaaaggggccaaatggaagcagcaagacctgagttcaaattaaatctgccaactaccagctcagtgaatctgggcgagtaacacaaaacttgagtgtccttacctgaaaaatagaggttagagggatgctatgtgccattgtgtgtgtgtgttgggggtggggattgggggtgatttgtgagcaattggaggtgagggtggagcccagtgcccagcacctatgcactggggacccaaaaaggagcatcttctcatgattttatgtatcagaaattgggatggcatgtcattgggacagcgtcttttttcttgtatggtggcacataaatacatgtgtcttataattaatggtattttagatttgacgaaatatggaatattacctgttgtgctgatcttgggcaaactataatatctctgggcaaaaatgtccccatctgaaaaacagggacaacgttcctccctcagccagccactatggggctaaaatgagaccacatctgtcaagggttttgccctcacctccctccctgctggayggcatccttggtrggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgwacttgcagtatctccacgacccgcccctgtgagtccccctccaggcaggtctatgaggggtgtggagggagggctgcccccgggagaagagagctaggtggtgatgagggctgaatcctccagccagggtgctcaacaagcctgagcttggggtaaaaggacacaaggccctccacaggccaggcctggcagccacagtctcaggtccctttgccatgcgcctccctctttccaggccaagggtccccagggcccagggccattccaacagacagtttggagcccaggaccctccattctccccaccccacttccacctttgggggtgtcggatttgaacaaatctcagaagcggcctcagagggagtcggcaagaatggagagcagggtccggtagggtgtgcagagggccacgtggcctatccactggggagggttccttgatctctggccaccagggctatctctgtggccttttggagcacctggtggtttggggcaggggttgaatttccaggcctaaaaccacacaggcctggccttgagtcctggctctgcgagtaatgcatggatgtaaacatggagacccaggaccttgcctcagtcttccgagtctggtgcctgcagtgtactgatrgtgtgagaccctactcctggaggatgggggacagaatctgatcgatcccctgggttggtgacttccctgtgcaatcaacggagaccagcaagggttggatttttaataaaccacttaactcctccgagtctcagtttccccctctatgaaatggggttgacagcattaataactacctcttgggtggttgtgagccttaactgaagtcataatatctcatgtttactgagcatgagctatgtgcaaagcctgttttgagagctttatgtggactaactcctttaattctcacaacaccctttaaggcacagatacaccacgttattccatccattttacaaatgaggaaactgaggcatggagcagttaagcatcttgcccaacattgccctccagtaagtgctggagctggaatttgcaccgtgcagtctggcttcatggcctgccctgtgaatcctgtaaaaattgtttgaaagacaccatgagtgtccaatcaacgttagctaatattctcagcccagtcatcagaccggcagaggcagccaccccactgtccccagggaggacacaaacatcctggcaccctctccactgcattctggagctgctttctaggcaggcagtgtgagctcagccccacgtagagcgggcagccgaggccttctgaggctatgtctctagcgaacaaggaccctcaatyccagcttccgccctgacggccagcacacagggacagccctttcattccgcttccacctgggggtgcaggcagagcagcagcgggggtaggcactgcccggagctcagaagtcctcctcagacaggtgccagtgcctccagaatgtggcagctcacaagcctcctgctgttcgtggccacctggggaatttccggcacaccagctcctcttggtaaggccaccccacccctaccccgggacccttgtggcctctacaaggcctggtggcatctgcccaggccttcacagcttccaccatctctctgagccctgggtgaggtgaggggcagatgggaatggcaggaatcaactgacaagtcccaggtaggccagctgccagagtgccacacaggggctgccagggcaggcatgcgtgatggcagggagccccgcgatgacctcctaaagctccctcctccacacggggatggtcacagagtcccctgggccttccctctccacccactcactccctcaactgtgaagaccccaggcccaggctaccgtccacactatccagcacagcctcccctactcaaatgcacactggcctcacggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgcctttycccccatcccttcttgctcacacccccaacttgatctctccctcctaactgtgccctgcacccaagasagacacttcacaragcccaggagacacctggggacccttcctgggtgataggtctgtctatcctccaggtgtccctgcccaaggggagaagcatggggaatacttggttgggggaggaraggaagactggggggatgtgtcaagatggggctgcaygtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaaggtaaagacactggctcaaggtcacacagagatcggggtggggttcacagggaggcctgtccatctcagagcaaggcttcgtcctccaactgccatctgcttcctggggaggaaaagagcagaggacccctgcgccaagccatgacctagaattagaatgagtcttgagggggcggagacaagaccttcccaggctctcccagctctgcttcctcagaccccctcatggccccagcccctcttaggcccctccaccaaggtgagctccccctccctccaaaaccagactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacagtaaggccaccatgggtccagaggatgaggctcaggggcgagctggtaaccagcaggggcctcgaggagcaggtggggactcaatgctgaggccctcttaggagttgtgggggtggctgagtggagcgattaggatgctggccctatgatgtcggccaggcacatgtgactgcaagaaacagaattcaggaagaagctccaggaaagagtgtggggtgaccctaggtggggactcccaccagccacagtgtaggtggttcagtccaccctccagccactgctgagcaccactgcctccccrtcccacctcacaaagaggggacctaaagaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtcctttggagcccccagagtcctgtggacgtggccctaggtagtagggtgagcttggtaacggggctggcttcctgagacaaggctcagacccgctctgtccctggggatcgcttcagccacyaggacctgaaaattgtgcacggcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacrccacagtgactttctgcaggcacatatctgatcacatcaagtccccaccgtgctcccacctcacccatggtctctcagccccagcaggccttggctggcctctctgatggagcaggcatcaggcacaggccgtgggtctcaacgtgggctgggtggtcctggaccagcagcagccgccgcagcagcaaccctggtacctggttaggaacgcagaccctctgcccccatcctcccaactctgaaaaacactggcttagggaaaggcgcgatgctcaggggtcccccaaagcccgcaggcagagggagtgatgggactggaaggaggccgagtgacttggtgagggattcgggtcccttgcatgccagaggctgctgtgggagcrgacagtcgcgagagcagcactgcagctgcatggggagagggtgttgctccagggacgtgggatggaggctgggcgcgggcgggtggcgctggagggcgggggaggggcagggagcaccagctcctagcagccaacgaccatcgggcgtcgatccctgtttgtctggaagccctcccctcccctgcccgctcacccgctgccctgccccacccgggcgcgccccctccgcacaccggctgcaggagcctgacgctgcccgctctctccgcagctggccttctggtccaagcacgtcggtgagtgcgttctagatccccggctggactaccggcgcccgcgcccctcgggatctctggccgctgaccccctaccccgccttgtgtcgcagacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcggtgagggggagaggtggatgctggcgggcggcggggcggggctggggccgggttgggggcgcggcaccagcaccagctgcccgcgccctcccctgcccgcagaggtgagcttcctcaattgctcgctggacaacggcggctgcacgcattactgcctagaggaggtgggctggcggcgctgtagctgtgcgcctggctacaagctgggggacgacctcctgcagtgtcaccccgcaggtgagaagcccccaatacatcgcccaggaatcacgctgggtgcggggtgggcaggcccctgacggggcgcggcgcggggggctcaggagggtttctagggagggagcgaggaacagagttgagccttggggcagcggcagacgcgccccaacaccggggccactgttagcgcaatcagcccgggagctgggcgcgccctccgctttccctgcttcctttcttcctggcgtccccgccttcctccgggcgccccctgcgcacctggggccacctcctggagcgcaagcccagtggtggctccgctccccagtctgagcgtatctggggcgaggcgtgcagcgtcctcctccatgtagcctggctgcgtttttctctgacgttgtccggcgtgcatcgcatttccctctttacccccttgcttccttgaggagagaacagaatcccgattctgccttcttctatattttcctttttatgcattttaatcaaatttatatatgtatgaaactttaaaaatcagagttttacaactyttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagttttattgtctcttctatttcccatctctttgactttgtgttttctttcagggaactttcttttttttctttttttttgagatggagtttcactcttgttgtcccaggctggagtgcaatgacgtgatctcagctcaccacaacctccgcctcctggattcaagcgattctcctgccgcagcctcccgagtagctgggattacaggcatgcgccaccacgcccagctaattttgtgtttttagtagagaaggggtttctccgtgttggtcaagctggtcttgaactcctgacctcaggtgatccacctgccttggcctcctaaagtgctgggattacaggcgtgagccaccgcgcccagcctctttcagggaactttctacaactttataattcaattcttctgcagaaaaaaatttttggccaggctcagtagctcagaccaataattccagcactttgagaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccktgggaaggccctgtcattggcagaaccccagatcgtgagggctttccttttaggctgctttctaagaggactcctccaagctcttggaggatggaagacgctcacccatggtgttcggcccctcagagcagggtggggcaggggagctggtgcctgtgcaggctgtggacatttgcatgactccctgtggtcagctaagagcaccactccttcctgaagcggggcctgaagtccctagtcagagcctctggttcaccttctgcaggcagggagaggggagtcmagtcagtgaggagggctttcgcagtttctcttacaaactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcagcttctggaattgttgctgttgtgccctttccattcttttgttatgatgcagctcccctgctgacgacgtcccattgctcttttaagtctagatatctggactgggcattcaaggcccattttgagcagagtcgggccgacctttcagccctcagttctccatggagtatgcgctctcttcttggcagggaggcctcacaaacatgccatgcctattgtaggagctctccaagaatgctcacctccttctccctgtaattcctttcctctgtgaggagctcagcagcatcccattatgagaccttactaatcccagggatcacccccaacagccctggggtacaatgagcttttaagaagtttaaccacctatgtaaggagacacaggcagtgggcgatgctgcctggcctgactcttgccattgggtggtactgtttgttgactgactgactgactgactggagggggtttgtaatttgtatctcagggattacccccaacagccctggggtacaatgagccttcaagaagtttaacaacctatgtaaggacacacagccagtgggtgatgctgcctggtctgactcttgccattcagtggcactgtttgttgactgactgactgactgactggctgactggagggggttcatagctaatattaatggagtggtctaagtatcattggttccttgaaccctgcactgtggcaaagtggcccacaggctggaggaggaccaagacaggagggcagtctcgggaggagtgcctggcaggcccctcaccacctctgcctacctcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtgggaggcgaggcagcaccggctgctcacgtgctgggtccgggatcactgagtccatcctggcagctatgctcagggtgcagaaaccgagagggaagcgctgccattgcgtttgggggatgatgaaggtgggggatgcttcagggaaagatggacgcaacctgaggggagaggagcagccagggtgggtgaggggaggggcatgggggcatggaggggtctgcaggagggagggttacagtttctaaaaagagctggaaagacactgctctgctggcgggattttaggcagaagccctgctgatgggagagggctaggagggagggccgggcctgagtacccctccagcctccacatgggaactgacacttactgggttcccctctctgccaggcatgggggagataggaaccaacaagtgggagtatttgccctggggactcagactctgcaagggtcaggaccccaaagacccggcagcccagtgggaccacagccaggacggcccttcaagataggggctgagggaggcccaaggggaacatccaggcagcctgggggccacaaagtcttcctggaagacacaaggcctggccaagcctctaaggatgagaggagctcgctgggcgatgttgggtgtggctgagggtgactgaaacagtatgaacagtgcaggaacagcatgggcaaaggcaggaagacaccctgggacaggctgacactgtaaaatgggcaaaaatagaaaacgccagaaagggcctaagcctatgcccatatgaccagggaacccaggaaagtgcatatgaaacccaggtgccctggactggaggctgtcaggaggcagccctgtgatgtcatcatcccaccccattccaggtggtcctgctggactcaaagaagaagctggcctgcggggcagtgctcatccacccctcctgggtgctgacagcggcccactgcatggatgagtccaagaagctccttgtcaggcttggtatgggctggagccaggcagaagggggctgccagaggcctgggtagggggaccaggcaggctgttcaggtttgggggaccccgctccccaggtgcttaagcaagaggcttcttgagctccacagaaggtgtttggggggaagaggcctatgtgcccccaccctgcccacccatgtacacccagtattttgcagtagggggttctctggtgccctcttcgaatctgggcacaggtacctgcacacacatgtttgtgaggggctacacagaccttcacctctccactcccactcatgaggagcaggctgtgtgggcctcagcacccttgggtgcagagaccagcaaggcctggcctcagggctgtgcctcccacagactgacagggatggagctgtacagagggagccctagcatctgccaaagccacaagctgcttccctagcaggctgggggcacctatgcattggccccgatctatggcaatttctggagggggggtctggctcaactctttatgccaaaaagaaggcaaagcatattgagaaaggccaaattcacatttcctacagcataatctatggccagtggccccccgtggggcttggcttagaattcccaggtgctcttcccagggaaccatcagtctggactgagaggaccttctctctcaggtgggacccggccctgtcctccctggcagtgccgtgttctgggggtcctcctctctgggtctcactgcccctggggtctctccagctacctttgctccaygttcctttgtggctctggtctgtgtctggggtttccaggggtctcgggcttccctgctgcccattccttctctggtctcacggctccgtgactcctgaaaaccaaccagcatcctaccyctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctcaggaaagtgccactggggagaggctccccgcagcccactctgactgtgccctctgccctgcaggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaagccactagagccttttccagggctttgggaagagcctgtgcaagccggggatgctgaaggtgaggcttgaccagctttccagctagcccagctatgaggtagacatgtttagctcatatcacagaggaggaaactgaggggtctgaaaggtttacatggtggagccaggattcaaatctaggtctgactccaaaacccaggtgcttttttctgttctccactgtcctggaggacagctgtttcgacggtgctcagtgtggaggccactattagctctgtagggaagcagccagagacccagaaagtgttggttcagcccagaatgagctcacagtgtcgcgggggaagctgtttaagaacaatgttacaccatcatgaacagcagtaagaaagaggctctggcttaacctggcctgataggcctaattgaatgagacagaaataagtcaaggatgctctgatttgaaatcatgaagtacctgatgaaaagaaatggtggtgagataaagctg

SEQ ID NO: 2 - DNA, 7199 bp, Homo sapiens
tagagaagcgagaccacatctctagtaaaaataaaaaaaaaatagctaggcgtggtggcacagtggcacgtacctttagtctcagctactcgggtggttgaggtgggagaatcacttgagcccgggaggtcaagcctacaattagctgtgattgcttcactgcactatagcctgggcaacagagctagaccctgtctcaaaaaaataataataaattttatatatatatatgaggatgaaattacatatgtattatttgaacagaagtgaaatcttttctttttttttttcaaaaaaaattttgccgcatgccccaggctaaaatgcagtggtgtgatctgggccctctgaaacctccacctcccgggttcaagggattctcatgcctcggtctcccaagtagctgggattacaggcatgcaccaccatgcccagctaatttttgtatttttcgtagagacgttcgccatattggccaggctggtctcaaactcctggcctcaagtgatctgcccacctcggcctcccaaagtgccagcagcatgctcggaggagtgactttaaagcttttctacttgcttcctagagtaagggacgcattttacactgctatccaaaactcatcatagaaacatacacacacaaaaccaaagcacacatatacaactgagcaaatatttcatgacataacactttctcttactaagggtgacgcgctgaaattttgtattctgtcctatttcattttttaaaaatggtaaccatgacctgctaaattgatttcattgtccactaataaattatgacctcagtttcaaaaagattgctttaggtaaccaatcatcttctgagatttatacagattgctcataattctctcctattttttaaaaacatgctgcagtgaactgctttacactcattttatgactacttctgagaccaagatcccggattatgtaattgttatttacttaaaattctggtaaaatgtagccattatactggaaaactaaattttaatcttggatctgtcaccaccatgatatataaactttgggcaagtccctgcacctctctggacctcaatctccccatcagcaacctgctgatcctactcccaggagtgtgctctaagttgaaagtagatgccccaccccctgagtcagcgccggcaggacttctcaccaagcccttctcccccttttccgctccctgttcctggttcctaggaagcagcccaaggagaagggaaaaggcaggtctgggcaggagggagcaatgaagggcggggcagagggagggcaggagggaggccggccccctagtaggaaatgagacacagtagaaataacactttataagcctcttcctcctcccatctcctggcctccttccatcctcctctgcccagactccgcccctcccagacggtcctcacttctcttttccctagactgcagccagcggagcccgcagccggcccgagccaggaacccaggtccggagcctcaacttcaggatgttgacaacattgctgccgatactgctgctgtctggctgggccttttgtagccaagacgcctcagatggtgagtcgggggcacatctcctgcctcaggatggttctggagaatctcagtctatctgggcacatggcaagaccacaggagagcttatctcacagcatctgtgtctgcagctggctagatctctctacagggcaggcagagtcttggggactggttcgtgtcccaaagccaaggtgagttagtacatttaagcccctgaaaagggggagatgaaagaggctaggggaaacaggatgactggaaacatgagaaagaaaccagcagagagggtaggagaatcagccccagggagaggggagaaaggggaactgagggtgatggtagataggggtacatctaggggagacgggaagaggctcagaagagaagagaaatggagggaatgggaagaccctgggaaaactgatggaagaagtgggggaagagtggggcagagagaggttaggggaggctagggaaaatggaaggagactggtcgcagctggtggaactggggagaaagagatgctgtgcctaatagaacttatgggcgatcaggctactgaagtggccctgtttaagcagaaaagggagttattaccctccattataattgcacaggggcctcctttcccctctctcacaatccccgtaacttcagtctccccctcagagaggcagcaaataataaccagtattcaatgagtgctcactatggttaatacatgtattgacccatttaacttgcacaaacccctaaaggtgggtaatattattactatctccattttatgaggaggaaactgggtcacagagtagttaaggaccatgtctagggttatccataaatatacttattcacatctgcagatacaaagcacaacttctcaaatgcaaacacagacaggacccactcacacacacagatttacaaccccggactcatccaaatgtgctctgggcatcaactctgtgccagcctcttttctgggtgtaggaagcagagattaccaagcatggttccatagcctagaggagtccagtgtggcctgtgtgtgtttggagacagccaggtagtatcccgtgagatacacactaatatatggtggtctgggatcactgaaacagacacactgtgtctcgtggggcatcagaaaaaaatttccaagaagagggcaactgagctgggtctttttttctttgcttttctttcttttttcttttttttttttttttttttttttgagatggagtcttgtgctgtcacccaggctggaatgcagtggcacaatttcagctaactgtaacctccaactcccaggttcaggcgattctcctgcctcagcctcctgagtagctgggactacaggcatgtaccaccacgcctggctaatatttgtayttttagtacagatggggtttcgccatgttggccaggctggtcttgaatccctgacctcaagtgatccgcccgcctcggcctcccaaagtrctgggattacaggcatgagccaccgcgcccagtctctgagctgggtcttaaatcatgaataaacttcgccaggcagaaaaagggaggcagagcaatcctgacatgctattcatgtgtcagccaaaggcagcatgaggaatcccaactagtttgatatataagcagcgggaagcggccagaaaaggcagcaggggccaggtctctagcagccttgaatgccaggctaaagactctggacttgatcctgtggggaggcagtgtagcagaatggctgagtgctggacttgactgcctacgtgcaaaccttggctctgctacactatctctgtctcagtttcscatgtagactggggttaataatagtagctattgcattaagccactggggaaaggcacaaagataataatgtatgtaaagcccattgcccaggttataataagcactgaatcgacattggctatgattatttttgattaatgaaggggagggggttatggcactggaagattttaagtaggaaaaggacatgatctcatccctgggtcaggtggaggtcggaatagagaacggggagatgaagtagaaagttactaccccagtctagatgagacggatgaatcctgaatcagggcagtggaagaggagatggagaacaggcgatggaattggaattttattcaggtcaggatttgttaaccatttgttccgttggttaacaggaaacggggggagggagagccgagggtgaaaaaggaggcagaaaggagtgtctcttccactgcaggcctcagtttcctcatctgtaaaacggagataataatccctgtcctgtcctcctggcagagttactgtcagcgtcaaacgggagaagcggtgggagggcacattatagtttatgaagggtcgagaaggcgggcggccagcctcgaggtagggggttattatcttccgctgcccgccgccccctcccacgccggcccaggctgaagytgactctgcccgcaggcctccaaagacttcatatgctccagatctcctacttccgcgacccctatcacgtgtggtaccagggcaacgcgtcgctggggggacacctaacgcacgtgctggaaggcccagacaccaacaccacgatcattcagctgcagcccttgcaggagcccgagagctgggcgcgcacgcagagtggcctgcagtcctacctgctccagttccacggcctcgtgcgcctggtgcaccaggagcggaccttggcctgtgagtaggcgcgcagcgggggcggggtctgggcggggctagtgggggcggggcctggcgggtgggggcggggcctggcggatggaggcgggctgggacttgcagggacccggcagccactggagctcggtggcgcctgggcctttgaagattgctgggtgggggctggagagaggcagttgtccccgctaagaaagccccgactcgggcggtcgtcctgctggcataacctcttgggatagaccctgttggaaggccctgacaccgtgacgtcgaaggtccccagaaaactcctcacccctcgcctcacagtcctccaactccttttcttcatagatctccgtccttcccttcccacagcccccagcacttcaccccccaccctccagccacttctcatacaagctgatgacttcgctcttagctccactcatgacccgaactcttcccccaaagaccccaagttcttctctcaaagccccactccttccccgtcacaaccctaactccttcttctcaaagaccccaatttcttttctcaaagcaccaagcaccactccgtcccccttcccccaccatcatggcctttaactcctttctctcctagtcccccaccccacccccyttttttttttttttttttttttttgagacggagtcttgctctgtcgtccaggctggagtgcagtggcgcgatctcggctcactgcaacttccgcctcccgggttcaagcgattctcctgcctcagcctcccaagcagctgggactacaggcacccgccaccacgcccggctaattttttgtatttttagtagagacggggtttcgccatgttggccaggctggtctcgaactcctgacctcaggcgatccacaagcctggcctcccaaagtgctgggattacaggcgtgagctgccgcccctgccccagcctcaccccctgttttttttttctattacagttgaacaaggcctgacaattcccttttttcatcacagtccctggccccttctttcttagcctctaacaggctaaccccaaacccctcctcacagccccaggcccttctccccatagttccctgacctagactcccctctcctcacagcactgactcttgccttctcatgttcttttccccttggtgggcctcgcccacacctggcaccctctctgcacagtcccctgaycctgactgtctatccacagttcctctgaccatccgctgcttcctgggctgtgagctgcctcccgagggctctagagcccatgtcttcttcgaagtggctgtgaatgggagctcctttgtgagtttccggccggagagagccttgtggcaggcagacacccaggtcacctccggagtggtcaccttcaccctgcagcagctcaatgcctacaaccgcactcggtatgaactgcgggaattcctggaggacacctgtgtgcagtatgtgcagaaacatatttccgcggaaaacacgaaaggtatgatgggacggggcccaggcctgcaagctggggagagggcgggttccagacaaatggatggacctgaaggatggatgcctagagcaacaagaggcccacagctgggggtttgggacagaacacacgcagcttcagtcagttggtaaacgggtccctttcctctggggcagaaacgctttggggtttgactcaaatcatggactccttgggggcctattcttcgggctaactctttgcatgttctgcagggagccaaacaagccgctcctacacttcgctggtcctgggcgtcctggtgggcagtttcatcattgctggtgtggctgtaggcatcttcctgtgcacaggtggacggcgatgttaattactctccagccccstcagaaggggctggattgatggaggctggcaagggaaagtttcagctcactgtgaagccagactccccaactgaaacaccagaaggtttggagtgacagctcctttcttctcccacatctgcccactgaagatttgagggaggggagatggagaggagaggtggacaaagtacttggtttgctaagaacctaagaacgtgtatgctttgctgaattagtctgataagtgaatgtttatctatctttgtggaaaacagataatggagttggggcaggaagcctatggcccatcctccaaagacagacagaatcacctgaggcgttcaaaagatataaccaaataaacaagtcatccacaatcaaaatacaacattcaatacttccaggtgtgtcagacttgggatgggacgctgatataatagggtagaaagaagtaacacgaagaagtggtggaaatgtaaaatccaagtcatatggcagtgatcaattattaatcaattaataatattaataaatttcttatatttaaggcattgttatctcctccactttgcaaaatttctggaaaagtaacctatacccatttcttctgcttccttatttctcactcattctttttttttttttttttttttttgagacagagtcttgctctgttgcctaggctggagtgcaatggtgtgatctcagctcactgcaacctctgcctcccggttcaagcaattctcctgcctcagcctcccaagcagctgggattacagatgcatgccaccacacccagctaatttttgtatttttagtagagatggggtttcaccacgttggccatcctgacctcgtgatccgcctacctcggcctccccaagtgctgggattagacgtgagccactgcgcctggtcttctcactcattcttagacccagtgcaatctgacttctctataaactactctgagatcaccagtaacctctaattgtcaaaccatcaccctacatggtatctg

SEQ ID NO: 3 - DNA, 400 bp, Homo sapiens
ttccttgatctctggccaccagggctatctctgtggccttttggagcacctggtggtttggggcaggggttgaatttccaggcctaaaaccacacaggcctggccttgagtcctggctctgcgagtaatgcatggatgtaaacatggagacccaggaccttgcctcagtcttccgagtctggtgcctgcagtgtactgatrgtgtgagaccctactcctggaggatgggggacagaatctgatcgatcccctgggttggtgacttccctgtgcaatcaacggagaccagcaagggttggatttttaataaaccacttaactcctccgagtctcagtttccccctctatgaaatggggttgacagcattaataactacctcttgggtggttgtgagcctta

SEQ ID NO: 4 - DNA, 400 bp, Homo sapiens
cccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgcctttycccccatcccttcttgctcacacccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggagaggaagactggg

SEQ ID NO: 5 - DNA, 400 bp, Homo sapiens
ggcccctaaagccacaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagasagacacttcacagagcccaggagacacctggggacccttcctgggtgataggtctgtctatcctccaggtgtccctgcccaaggggagaagcatggggaatacttggttgggggaggagaggaagactggggggatgtgtcaagatggggctgcacgtggtgtactggcagaagagtgagaggatttaacttggcagcc

SEQ ID NO: 6 - DNA, 400 bp, Homo sapiens
acaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacaragcccaggagacacctggggacccttcctgggtgataggtctgtctatcctccaggtgtccctgcccaaggggagaagcatggggaatacttggttgggggaggaraggaagactggggggatgtgtcaagatggggctgcaygtggtgtactggcagaagagtgagaggatttaacttggcagcctttacagcagcag

SEQ ID NO: 7 - DNA, 400 bp, Homo sapiens
ggagttgtgggggtggctgagtggagcgattaggatgctggccctatgatgtcggccaggcacatgtgactgcaagaaacagaattcaggaagaagctccaggaaagagtgtggggtgaccctaggtggggactcccaccagccacagtgtaggtggttcagtccaccctccagccactgctgagcaccactgcctccccrtcccacctcacaaagaggggacctaaagaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtc

SEQ ID NO: 8 - DNA, 400 bp, Homo sapiens
tcagccacyaggacctgaaaattgtgcacggcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacrccacagtgactttctgcaggcacatatctgatcacatcaagtccccaccgtgctcccacctcacccatggtctctcagccccagcaggccttggctggcctctctgatggagcaggcatcaggcacaggccgtgggtctcaacgtgggctgggtggtcctggaccagcagcagccgccgcagcagcaaccctggtacct

SEQ ID NO: 9 - DNA, 400 bp, Homo sapiens
cagcaaccctggtacctggttaggaacgcagaccctctgcccccatcctcccaactctgaaaaacactggcttagggaaaggcgcgatgctcaggggtcccccaaagcccgcaggcagagggagtgatgggactggaaggaggccgagtgacttggtgagggattcgggtcccttgcatgccagaggctgctgtgggagcrgacagtcgcgagagcagcactgcagctgcatggggagagggtgttgctccagggacgtgggatggaggctgggcgcgggcgggtggcgctggagggcgggggaggggcagggagcaccagctcctagcagccaacgaccatcgggcgtcgatccctgtttgtctggaagccctcccctcccctgcccgctcacccgctg

SEQ ID NO: 10 - DNA, 400 bp, Homo sapiens
ggcccctgacggggcgcggcgcggggggctcaggagggtttctagggagggagcgaggaacagagttgagccttggggcagcggcagacgcgccccaacaccggggccactgttagcgcaatcagcccgggagctgggcgcgccctccgctttccctgcttcctttcttcctggcgtccccgccttcctccgggcgccccctgcgcacctggggccacctcctggagcgcaagcccagtggtggctccgctccccagtctgagcgtatctggggcgaggcgtgcagcgtcctcctccatgtagcctggctgcgtttttctctgacgttgtccggcgtgcatcgcatttccctctttacccccttgcttccttgaggagagaacagaatcccgattctgcc

SEQ ID NO: 11 - DNA, 400 bp, Homo sapiens
cgtgcagcgtcctcctccatgtagcctggctgcgtttttctctgacgttgtccggcgtgcatcgcatttccctctttacccccttgcttccttgaggagagaacagaatcccgattctgccttcttctatattttcctttttatgcattttaatcaaatttatatatgtatgaaactttaaaaatcagagttttacaactyttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagtttt

SEQ ID NO: 12 - DNA, 400 bp, Homo sapiens
gaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccktgggaaggccctgtcattggcagaaccccagatcgtgagggctttccttttaggctgctttctaagaggactcctccaagctcttggaggatggaagacgctcacccatggtgttcggcccctcagagcagggtggggcaggggagctggtgcctgtgcaggctgtggacatttgcatgactccctgtggtcagctaag

SEQ ID NO: 13 - DNA, 400 bp, Homo sapiens
cttggaggatggaagacgctcacccatggtgttcggcccctcagagcagggtggggcaggggagctggtgcctgtgcaggctgtggacatttgcatgactccctgtggtcagctaagagcaccactccttcctgaagcggggcctgaagtccctagtcagagcctctggttcaccttctgcaggcagggagaggggagtcmagtcagtgaggagggctttcgcagtttctcttacaaactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcag

SEQ ID NO: 14 - DNA, 400 bp, Homo sapiens
ctccctggcagtgccgtgttctgggggtcctcctctctgggtctcactgcccctggggtctctccagctacctttgctccaygttcctttgtggctctggtctgtgtctggggtttccaggggtctcgggcttccctgctgcccattccttctctggtctcacggctccgtgactcctgaaaaccaaccagcatcctaccyctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctcaggaaagtgccactgggg

SEQ ID NO: 15 - DNA, 401 bp, Homo sapiens
tgcttttctttcttttttcttttttttttttttttttttttttgagatggagtcttgtgctgtcacccaggctggaatgcagtggcacaatttcagctaactgtaacctccaactcccaggttcaggcgattctcctgcctcagcctcctgagtagctgggactacaggcatgtaccaccacgcctggctaatatttgtayttttagtacagatggggtttcgccatgttggccaggctggtcttgaatccctgacctcaagtgatccgcccgcctcggcctcccaaagtgctgggattacaggcatgagccaccgcgcccagtctctgagctgggtcttaaatcatgaataaacttcgccaggcagaaaaagggaggcagagcaatcctgacatgctatt

SEQ ID NO: 16 - DNA, 400 bp, Homo sapiens
tttcagctaactgtaacctccaactcccaggttcaggcgattctcctgcctcagcctcctgagtagctgggactacaggcatgtaccaccacgcctggctaatatttgtacttttagtacagatggggtttcgccatgttggccaggctggtcttgaatccctgacctcaagtgatccgcccgcctcggcctcccaaagtrctgggattacaggcatgagccaccgcgcccagtctctgagctgggtcttaaatcatgaataaacttcgccaggcagaaaaagggaggcagagcaatcctgacatgctattcatgtgtcagccaaaggcagcatgaggaatcccaactagtttgatatataagcagcgggaagcggccagaaaaggcagcaggggccagg

SEQ ID NO: 17 - DNA, 400 bp, Homo sapiens
atcccaactagtttgatatataagcagcgggaagcggccagaaaaggcagcaggggccaggtctctagcagccttgaatgccaggctaaagactctggacttgatcctgtggggaggcagtgtagcagaatggctgagtgctggacttgactgcctacgtgcaaaccttggctctgctacactatctctgtctcagtttcscatgtagactggggttaataatagtagctattgcattaagccactggggaaaggcacaaagataataatgtatgtaaagcccattgcccaggttataataagcactgaatcgacattggctatgattatttttgattaatgaaggggagggggttatggcactggaagattttaagtaggaaaaggacatgatctcatc

SEQ ID NO: 18 - DNA, 400 bp, Homo sapiens
agtttcctcatctgtaaaacggagataataatccctgtcctgtcctcctggcagagttactgtcagcgtcaaacgggagaagcggtgggagggcacattatagtttatgaagggtcgagaaggcgggcggccagcctcgaggtagggggttattatcttccgctgcccgccgccccctcccacgccggcccaggctgaagytgactctgcccgcaggcctccaaagacttcatatgctccagatctcctacttccgcgacccctatcacgtgtggtaccagggcaacgcgtcgctggggggacacctaacgcacgtgctggaaggcccagacaccaacaccacgatcattcagctgcagcccttgcaggagcccgagagctgggcgcgcacgcagagtgg

SEQ ID NO: 19 - DNA, 400 bp, Homo sapiens
ctccactcatgacccgaactcttcccccaaagaccccaagttcttctctcaaagccccactccttccccgtcacaaccctaactccttcttctcaaagaccccaatttcttttctcaaagcaccaagcaccactccgtcccccttcccccaccatcatggcctttaactcctttctctcctagtcccccaccccacccccyttttttttttttttttttttttttgagacggagtcttgctctgtcgtccaggctggagtgcagtggcgcgatctcggctcactgcaacttccgcctcccgggttcaagcgattctcctgcctcagcctcccaagcagctgggactacaggcacccgccaccacgcccggctaattttttgtatttttagtagagacggg

SEQ ID NO: 20 - DNA, 400 bp, Homo sapiens
tcatcacagtccctggccccttctttcttagcctctaacaggctaaccccaaacccctcctcacagccccaggcccttctccccatagttccctgacctagactcccctctcctcacagcactgactcttgccttctcatgttcttttccccttggtgggcctcgcccacacctggcaccctctctgcacagtcccctgaycctgactgtctatccacagttcctctgaccatccgctgcttcctgggctgtgagctgcctcccgagggctctagagcccatgtcttcttcgaagtggctgtgaatgggagctcctttgtgagtttccggccggagagagccttgtggcaggcagacacccaggtcacctccggagtggtcaccttcaccctgcagcagc

SEQ ID NO: 21 - DNA, 400 bp, Homo sapiens
ggggtttgactcaaatcatggactccttgggggcctattcttcgggctaactctttgcatgttctgcagggagccaaacaagccgctcctacacttcgctggtcctgggcgtcctggtgggcagtttcatcattgctggtgtggctgtaggcatcttcctgtgcacaggtggacggcgatgttaattactctccagccccstcagaaggggctggattgatggaggctggcaagggaaagtttcagctcactgtgaagccagactccccaactgaaacaccagaaggtttggagtgacagctcctttcttctcccacatctgcccactgaagatttgagggaggggagatggagaggagaggtggacaaagtacttggtttgctaagaacctaagaacgt

SEQ ID NO: 22 - DNA, 30 bp, Homo sapiens
gcagaggacccttcsgccctctgggcagcc

SEQ ID NO: 23 - DNA, 28 bp, Homo sapiens
tcctctggaaacagsccctccttcatat

SEQ ID NO: 24 - DNA, 25 bp, Homo sapiens
cacagccaaaaaagygtgaacacaa

SEQ ID NO: 25 - DNA, 32 bp, Homo sapiens
tggggaactcactcwatttccatgctatctct

SEQ ID NO: 26 - DNA, 26 bp, Homo sapiens
tttcagttatatccrtatttccttga

SEQ ID NO: 27 - DNA, 35 bp, Homo sapiens
tagggtcattatttygaaactaaaagcagacctgg

SEQ ID NO: 28 - DNA, 29 bp, Homo sapiens
acctctcgtgtatayactctggtagggcc

SEQ ID NO: 29 - DNA, 30 bp, Homo sapiens
gaagcgacccagctyacctcagcagcttca

SEQ ID NO: 30 - DNA, 34 bp, Homo sapiens
gacaaatctcttgamatcagtatatggctggttt

SEQ ID NO: 31 - DNA, 35 bp, Homo sapiens
ttgcagttttattaygatgtagttaggtgtagatt

SEQ ID NO: 32 - DNA, 35 bp, Homo sapiens
ttttgtgtataatamgtacatatgaaaaacttaaa

SEQ ID NO: 33 - DNA, 35 bp, Homo sapiens
tatactctgcagtgrgggagatgggataatggaca

SEQ ID NO: 34 - DNA, 35 bp, Homo sapiens
ggaacataagatgartaaggcatggattctgcatt

SEQ ID NO: 35 - DNA, 35 bp, Homo sapiens
agatgcagggcaggygccccagtgcttcttgggaa

SEQ ID NO: 36 - DNA, 35 bp, Homo sapiens
cacccagcatgtgaytccactatctgaagacacag

SEQ ID NO: 37 - DNA, 35 bp, Homo sapiens
ctgacagagtggttmtaaggagagaaaccgaatag

SEQ ID NO: 38 - DNA, 35 bp, Homo sapiens
tctttctcctgggtrtcctgctagagtctgagcca

SEQ ID NO: 39 - DNA, 35 bp, Homo sapiens
agagatttcctctcygggcctaaaggtcaaacaac

SEQ ID NO: 40 - DNA, 35 bp, Homo sapiens
gtaagaattgcgggragcgcggctctagctcagct

SEQ ID NO: 41 - DNA, 35 bp, Homo sapiens
aaagggaaaggaccsggttcacgcttcccattccc

SEQ ID NO: 42 - DNA, 35 bp, Homo sapiens
taaacaagtcatccmcaatcaaaatacaacattca

SEQ ID NO: 43 - DNA, 35 bp, Homo sapiens
cccacccaaacaaamaacaaaacccattattttat

SEQ ID NO: 44 - DNA, 35 bp, Homo sapiens
agattagatttggtytgtggaattccagggaacag

SEQ ID NO: 45 - DNA, 35 bp, Homo sapiens
acattaaaaaaaaawtatttgtttagggtctgtcc

SEQ ID NO: 46 - DNA, 13869 bp, Homo sapiens
gctctctaactcacagcgagctcgctgcccaaagtcctgctccgggggcttcctgggtggacctgaccgcgttcgggtgcacgtggggcgactcacacctgacaagtaaagcgggtgaggccgcgcctgtgaagggcgcctggctcctccgcaggacggtgcggcgcggcgcccccggctggaaccaggtgtaactgcagagaccctgggatcgcaggaacggctggcggcaggactgtccctacctcgagaaggtgacggggtttcctgcgctgccagccgatgaggcggccgtgacgcagcccgccgtgcagagtccccgtcggccgacaggcgtgcagagctctgcagaggacccttccgccctctgggcagcctgccaagccgtggcacccccaacccccagcactgggcacttgggagcattgcagccgccctggctcgtaccggtgccggtgctttgggcacctgggctggtttggacatgggtgccccgggcagagtccatttatgcaggtcagaatcagtgtgtggagcctgcatagacttgccctggagcggctgcctgtgctggggtggggaggagtagagggcgagaagttggtggggaagggaagcggcgccaaaagaatacccacaacatcttgcacctggaaggcaaagcagagggcagtgatctctgcagacttgcgggggcgacgcctgaagcaaacagggacatacaagctggtgccttctgtggttgtgcatggggtcttcatgcttcctgtctgagttcccagaagcttgtctctgcttttctaggcagctgccacagcctgtcacaaacagctcctggttctccacttctcatagtctcgatttcaaaatccattgcctcaccctccacctcctctccacctccacccctcctagcacctcctgactgcttgtgttctgtgtctccccactgtctcccaacctggggtggggttgggggggatgtctttcctcctgtctgctctttgatgtccagctgaagtgtcacctcctacaggcagcctcccctggctatgccagcttgtactgattgccctctcctctgaattctgtaagcatttcctatgtgtacctgcccctgggcaaggtgggcctgacttgttagagtgttagagttttaccctgttcctctaggagggcctggtaccaccacagcccagcatggtgtggtgcctcagcaggaggcatctggttacaatcaacacaagctgttccagccaatttaaagaaacttcaggaggaatagggttttaggagggcatggggaccctcctgcacccgaagccaggatgtgccaccaatcataaggaggcaggggcctccttccgctgctccctgggactctcyaggtgtccgtggcctcagcccccctctgcacacctgcatcttccttctcatcagcttcctctgctttaagcgtaaacatggatgcccaggacctggcctcaatcttccgagtctggtacttatggtgtactgacagtgtgagaccctactcctctgatcaatcccctgggttggtgacttccctgtgcaatcaatggaagccagcgaggcagggtcacatgccccgtttagaggtgcagacttggagaaggaacgtgggcaagtcttcccaggaacaggtagggcagggaggaaaggggggcatctctggtgcagcccggttcggagcaggaagacgcttaataaatgctgatagactgcaggacacaggcaaaggtgctgagctggaccctttatttctgcccttctcccttctggcaccccggccaggaaattgctgcagcctttctggaatcccgttcatttttcttactggtccacaaaaggggccaaatggaagcagcaagacctgagttcaaattaaatctgccaactaccagctcagtgaatctgggcgagtaacacaaaacttgagtgtccttacctgaaaaatagaggttagagggatgctatgtgccattgtgtgtgtgtgttgggggtggggattgggggtgatttgtgagcaattggaggtgagggtggagcccagtgcccagcacctatgcactggggacccaaaaaggagcatcttctcatgattttatgtatcagaaattgggatggcatgtcattgggacagcgtcttttttcttgtatggtggcacataaatacatgtgtcttataattaatggtattttagatttgacgaaatatggaatattacctgttgtgctgatcttgggcaaactataatatctctgggcaaaaatgtccccatctgaaaaacagggacaacgttcctccctcagccagccactatggggctaaaatgagaccacatctgtcaagggttttgccctcacctccctccctgctggayggcatccttggtrggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgwacttgcagtatctccacgacccgcccctgtgagtccccctccaggcaggtctatgaggggtgtggagggagggctgcccccgggagaagagagctaggtggtgatgagggctgaatcctccagccagggtgctcaacaagcctgagcttggggtaaaaggacacaaggccctccacaggccaggcctggcagccacagtctcaggtccctttgccatgcgcctccctctttccaggccaagggtccccagggcccagggccattccaacagacagtttggagcccaggaccctccattctccccaccccacttccacctttgggggtgtcggatttgaacaaatctcagaagcggcctcagagggagtcggcaagaatggagagcagggtccggtagggtgtgcagagggccacgtggcctatccactggggagggttccttgatctctggccaccagggctatctctgtggccttttggagcacctggtggtttggggcaggggttgaatttccaggcctaaaaccacacaggcctggccttgagtcctggctctgcgagtaatgcatggatgtaaacatggagacccaggaccttgcctcagtcttccgagtctggtgcctgcagtgtactgatrgtgtgagaccctactcctggaggatgggggacagaatctgatcgatcccctgggttggtgacttccctgtgcaatcaacggagaccagcaagggttggatttttaataaaccacttaactcctccgagtctcagtttccccctctatgaaatggggttgacagcattaataactacctcttgggtggttgtgagccttaactgaagtcataatatctcatgtttactgagcatgagctatgtgcaaagcctgttttgagagctttatgtggactaactcctttaattctcacaacaccctttaaggcacagatacaccacgttattccatccattttacaaatgaggaaactgaggcatggagcagttaagcatcttgcccaacattgccctccagtaagtgctggagctggaatttgcaccgtgcagtctggcttcatggcctgccctgtgaatcctgtaaaaattgtttgaaagacaccatgagtgtccaatcaacgttagctaatattctcagcccagtcatcagaccggcagaggcagccaccccactgtccccagggaggacacaaacatcctggcaccctctccactgcattctggagctgctttctaggcaggcagtgtgagctcagccccacgtagagcgggcagccgaggccttctgaggctatgtctctagcgaacaaggaccctcaatyccagcttccgccctgacggccagcacacagggacagccctttcattccgcttccacctgggggtgcaggcagagcagcagcgggggtaggcactgcccggagctcagaagtcctcctcagacaggtgccagtgcctccagaatgtggcagctcacaagcctcctgctgttcgtggccacctggggaatttccggcacaccagctcctcttggtaaggccaccccacccctaccccgggacccttgtggcctctacaaggcctggtggcatctgcccaggccttcacagcttccaccatctctctgagccctgggtgaggtgaggggcagatgggaatggcaggaatcaactgacaagtcccaggtaggccagctgccagagtgccacacaggggctgccagggcaggcatgcgtgatggcagggagccccgcgatgacctcctaaagctccctcctccacacggggatggtcacagagtcccctgggccttccctctccacccactcactccctcaactgtgaagaccccaggcccaggctaccgtccacactatccagcacagcctcccctactcaaatgcacactggcctcacggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgcctttycccccatcccttcttgctcacacccccaacttgatctctccctcctaactgtgccctgcacccaagasagacacttcacaragcccaggagacacctggggacccttcctgggtgataggtctgtctatcctccaggtgtccctgcccaaggggagaagcatggggaatacttggttgggggaggaraggaagactggggggatgtgtcaagatggggctgcaygtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaaggtaaagacactggctcaaggtcacacagagatcggggtggggttcacagggaggcctgtccatctcagagcaaggcttcgtcctccaactgccatctgcttcctggggaggaaaagagcagaggacccctgcgccaagccatgacctagaattagaatgagtcttgagggggcggagacaagaccttcccaggctctcccagctctgcttcctcagaccccctcatggccccagcccctcttaggcccctccaccaaggtgagctccccctccctccaaaaccagactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacagtaaggccaccatgggtccagaggatgaggctcaggggcgagctggtaaccagcaggggcctcgaggagcaggtggggactcaatgctgaggccctcttaggagttgtgggggtggctgagtggagcgattaggatgctggccctatgatgtcggccaggcacatgtgactgcaagaaacagaattcaggaagaagctccaggaaagagtgtggggtgaccctaggtggggactcccaccagccacagtgtaggtggttcagtccaccctccagccactgctgagcaccactgcctccccrtcccacctcacaaagaggggacctaaagaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtcctttggagcccccagagtcctgtggacgtggccctaggtagtagggtgagcttggtaacggggctggcttcctgagacaaggctcagacccgctctgtccctggggatcgcttcagccacyaggacctgaaaattgtgcacggcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacrccacagtgactttctgcaggcacatatctgatcacatcaagtccccaccgtgctcccacctcacccatggtctctcagccccagcaggccttggctggcctctctgatggagcaggcatcaggcacaggccgtgggtctcaacgtgggctgggtggtcctggaccagcagcagccgccgcagcagcaaccctggtacctggttaggaacgcagaccctctgcccccatcctcccaactctgaaaaacactggcttagggaaaggcgcgatgctcaggggtcccccaaagcccgcaggcagagggagtgatgggactggaaggaggccgagtgacttggtgagggattcgggtcccttgcatgccagaggctgctgtgggagcrgacagtcgcgagagcagcactgcagctgcatggggagagggtgttgctccagggacgtgggatggaggctgggcgcgggcgggtggcgctggagggcgggggaggggcagggagcaccagctcctagcagccaacgaccatcgggcgtcgatccctgtttgtctggaagccctcccctcccctgcccgctcacccgctgccctgccccacccgggcgcgccccctccgcacaccggctgcaggagcctgacgctgcccgctctctccgcagctggccttctggtccaagcacgtcggtgagtgcgttctagatccccggctggactaccggcgcccgcgcccctcgggatctctggccgctgaccccctaccccgccttgtgtcgcagacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcggtgagggggagaggtggatgctggcgggcggcggggcggggctggggccgggttgggggcgcggcaccagcaccagctgcccgcgccctcccctgcccgcagaggtgagcttcctcaattgctcgctggacaacggcggctgcacgcattactgcctagaggaggtgggctggcggcgctgtagctgtgcgcctggctacaagctgggggacgacctcctgcagtgtcaccccgcaggtgagaagcccccaatacatcgcccaggaatcacgctgggtgcggggtgggcaggcccctgacggggcgcggcgcggggggctcaggagggtttctagggagggagcgaggaacagagttgagccttggggcagcggcagacgcgccccaacaccggggccactgttagcgcaatcagcccgggagctgggcgcgccctccgctttccctgcttcctttcttcctggcgtccccgccttcctccgggcgcccctgcgcacctggggccacctcctggagcgcaagcccagtggtggctccgctccccagtctgagcgtatctggggcgaggcgtgcagcgtcctcctccatgtagcctggctgcgtttttctctgacgttgtccggcgtgcatcgcatttccctctttacccccttgcttccttgaggagagaacagaatcccgattctgccttcttctatattttcctttttatgcattttaatcaaatttatatatgtatgaaactttaaaaatcagagttttacaactyttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagttttattgtctcttctatttcccatctctttgactttgtgttttctttcagggaactttcttttttttctttttttttgagatggagtttcactcttgttgtcccaggctggagtgcaatgacgtgatctcagctcaccacaacctccgcctcctggattcaagcgattctcctgccgcagcctcccgagtagctgggattacaggcatgcgccaccacgcccagctaattttgtgtttttagtagagaaggggtttctccgtgttggtcaagctggtcttgaactcctgacctcaggtgatccacctgccttggcctcctaaagtgctgggattacaggcgtgagccaccgcgcccagcctctttcagggaactttctacaactttataattcaattcttctgcagaaaaaaatttttggccaggctcagtagctcagaccaataattccagcactttgagaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccktgggaaggccctgtcattggcagaaccccagatcgtgagggctttccttttaggctgctttctaagaggactcctccaagctcttggaggatggaagacgctcacccatggtgttcggcccctcagagcagggtggggcaggggagctggtgcctgtgcaggctgtggacatttgcatgactccctgtggtcagctaagagcaccactccttcctgaagcggggcctgaagtccctagtcagagcctctggttcaccttctgcaggcagggagaggggagtcmagtcagtgaggagggctttcgcagtttctcttacaaactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcagcttctggaattgttgctgttgtgccctttccattcttttgttatgatgcagctcccctgctgacgacgtcccattgctcttttaagtctagatatctggactgggcattcaaggcccattttgagcagagtcgggccgacctttcagccctcagttctccatggagtatgcgctctcttcttggcagggaggcctcacaaacatgccatgcctattgtaggagctctccaagaatgctcacctccttctccctgtaattcctttcctctgtgaggagctcagcagcatcccattatgagaccttactaatcccagggatcacccccaacagccctggggtacaatgagcttttaagaagtttaaccacctatgtaaggagacacaggcagtgggcgatgctgcctggcctgactcttgccattgggtggtactgtttgttgactgactgactgactgactggagggggtttgtaatttgtatctcagggattacccccaacagccctggggtacaatgagccttcaagaagtttaacaacctatgtaaggacacacagccagtgggtgatgctgcctggtctgactcttgccattcagtggcactgtttgttgactgactgactgactgactggctgactggagggggttcatagctaatattaatggagtggtctaagtatcattggttccttgaaccctgcactgtggcaaagtggcccacaggctggaggaggaccaagacaggagggcagtctcgggaggagtgcctggcaggcccctcaccacctctgcctacctcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtgggaggcgaggcagcaccggctgctcacgtgctgggtccgggatcactgagtccatcctggcagctatgctcagggtgcagaaaccgagagggaagcgctgccattgcgtttgggggatgatgaaggtgggggatgcttcagggaaagatggacgcaacctgaggggagaggagcagccagggtgggtgaggggaggggcatgggggcatggaggggtctgcaggagggagggttacagtttctaaaaagagctggaaagacactgctctgctggcgggattttaggcagaagccctgctgatgggagagggctaggagggagggccgggcctgagtacccctccagcctccacatgggaactgacacttactgggttcccctctctgccaggcatgggggagataggaaccaacaagtgggagtatttgccctggggactcagactctgcaagggtcaggaccccaaagacccggcagcccagtgggaccacagccaggacggcccttcaagataggggctgagggaggcccaaggggaacatccaggcagcctgggggccacaaagtcttcctggaagacacaaggcctggccaagcctctaaggatgagaggagctcgctgggcgatgttgggtgtggctgagggtgactgaaacagtatgaacagtgcaggaacagcatgggcaaaggcaggaagacaccctgggacaggctgacactgtaaaatgggcaaaaatagaaaacgccagaaagggcctaagcctatgcccatatgaccagggaacccaggaaagtgcatatgaaacccaggtgccctggactggaggctgtcaggaggcagccctgtgatgtcatcatcccaccccattccaggtggtcctgctggactcaaagaagaagctggcctgcggggcagtgctcatccacccctcctgggtgctgacagcggcccactgcatggatgagtccaagaagctccttgtcaggcttggtatgggctggagccaggcagaagggggctgccagaggcctgggtagggggaccaggcaggctgttcaggtttgggggaccccgctccccaggtgcttaagcaagaggcttcttgagctccacagaaggtgtttggggggaagaggcctatgtgcccccaccctgcccacccatgtacacccagtattttgcagtagggggttctctggtgccctcttcgaatctgggcacaggtacctgcacacacatgtttgtgaggggctacacagaccttcacctctccactcccactcatgaggagcaggctgtgtgggcctcagcacccttgggtgcagagaccagcaaggcctggcctcagggctgtgcctcccacagactgacagggatggagctgtacagagggagccctagcatctgccaaagccacaagctgcttccctagcaggctgggggcacctatgcattggccccgatctatggcaatttctggagggggggtctggctcaactctttatgccaaaaagaaggcaaagcatattgagaaaggccaaattcacatttcctacagcataatctatggccagtggccccccgtggggcttggcttagaattcccaggtgctcttcccagggaaccatcagtctggactgagaggaccttctctctcaggtgggacccggccctgtcctccctggcagtgccgtgttctgggggtcctcctctctgggtctcactgcccctggggtctctccagctacctttgctccaygttcctttgtggctctggtctgtgtctggggtttccaggggtctcgggcttccctgctgcccattccttctctggtctcacggctccgtgactcctgaaaaccaaccagcatcctaccyctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctcaggaaagtgccactggggagaggctccccgcagcccactctgactgtgccctctgccctgcaggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaagccactagagccttttccagggctttgggaagagcctgtgcaagccggggatgctgaaggtgaggcttgaccagctttccagctagcccagctatgaggtagacatgtttagctcatatcacagaggaggaaactgaggggtctgaaaggtttacatggtggagccaggattcaaatctaggtctgactccaaaacccaggtgcttttttctgttctccactgtcctggaggacagctgtttcgacggtgctcagtgtggaggccactattagctctgtagggaagcagccagagacccagaaagtgttggttcagcccagaatgagctcacagtgtcgcgggggaagctgtttaagaacaatgttacaccatcatgaacagcagtaagaaagaggctctggcttaacctggcctgataggcctaattgaatgagacagaaataagtcaaggatgctctgatttgaaatcatgaagtacctgatgaaaagaaatggtggtgagataaagctg

SEQ ID NO: 47 - DNA, 399 bp, Homo sapiens
ggcccctgacggggcgcggcgcggggggctcaggagggtttctagggagggagcgaggaacagagttgagccttggggcagcggcagacgcgccccaacaccggggccactgttagcgcaatcagcccgggagctgggcgcgccctccgctttccctgcttcctttcttcctggcgtccccgccttcctccgggcgcccctgcgcacctggggccacctcctggagcgcaagcccagtggtggctccgctccccagtctgagcgtatctggggcgaggcgtgcagcgtcctcctccatgtagcctggctgcgtttttctctgacgttgtccggcgtgcatcgcatttccctctttacccccttgcttccttgaggagagaacagaatcccgattctgcc

(19) United States
(12) Patent Application Publication
(10) Pub. No.: US 2008/0026371 A1
(43) Pub. Date: Jan. 31, 2008

(54) Protein C And Endothelial Protein C Receptor Polymorphisms As Indicators Of Subject Outcome

(76) Inventors: James Russell, Vancouver (CA); Keith R. Walley, Vancouver (CA)

Correspondence Address: BROWDY AND NEIMARK, P.L.L.C.;624 NINTH STREET, NW, SUITE 300, WASHINGTON, DC, 20001-5303, US

(21) Appl. No.: 10/591,263

(22) PCT Filed: Mar. 18, 2005
(86) PCT No.: PCT/CA05/00409

§ 371 Date: Jul. 11, 2007

Related U.S. Application Data
(60) Provisional application No. 60/553,955, filed on Mar. 18, 2004.
(60) Provisional application No. 60/616,640, filed on Oct. 8, 2004.
(60) Provisional application No. 60/632,934, filed on Dec. 6, 2004.

(30) Foreign Application Priority Data
Oct. 8, 2004 (CA) 2479968

(51) Int. Cl. C12Q 1/68; C07H 21/04; G06G 7/48

(52) U.S. Cl. 435/6; 536/23.5; 703/11

(57) ABSTRACT

The invention provides methods and kits for obtaining a prognosis for a subject having or at risk of developing an inflammatory condition and for identifying subjects having a greater benefit from treatment with an anti-inflammatory agent or an anti-coagulant agent. The method generally comprises determining a protein C and/or EPCR genotype(s) of a subject for a polymorphisms in the these genes, comparing the determined genotype with known genotypes for the polymorphism that correspond with the ability of the subject to recover from the inflammatory condition and identifying subjects based on their prognosis. The invention also provides for methods of identifying potential subjects having an inflammatory condition who are more likely to benefit from treatment with an anti-inflammatory agent or anti-coagulant agent and subsequent to treatment recover from the inflammatory condition. The invention also provides for methods of treating such subjects with an anti-inflammatory agent or anti-coagulant agent based on the subject's genotype.

### FIELD OF TEE INVENTION

The field of the invention relates to the assessment and/or treatment of subjects with an inflammatory condition.

### BACKGROUND OF THE INVENTION

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit Care Med (1996) 24:381-4; STUBER F. et al. Journal of Inflammation (1996) 46:42-50; and WEITKAMP J H. et al. Infection (2000) 28:92-6). Furthermore, septic and non-septic stimuli such as bacterial endotoxin and cardiopulmonary bypass (CPB), respectively, activate the coagulation system and trigger a systemic inflammatory response syndrome (SIRS); Protein C and-endothelial cell protein C receptor (EPCR) both play a role in the inflammatory response.

Protein C, when activated to form activated protein C (APC), plays a major role in three biological processes or conditions: coagulation, fibrinolysis and inflammation. Acute inflammatory states decrease levels of the free form of protein S, which decreases APC function because free protein S is an important co-factor for APC. Sepsis, acute inflammation and cytokines decrease thrombomodulin expression on endothelial cells resulting in decreased APC activity or levels. Septic shock also increases circulating levels of thrombomodulin, which is related to increased cleavage of endothelial cell thrombomodulin. Another mechanism for decreased APC function in sepsis is that endotoxin and cytokines, such as TNF-α, down-regulate endothelial cell protein C receptor (EPCR) expression, thereby decreasing activation of protein C to APC. Severe septic states such as meningococcemia, also result in protein C consumption. Depressed protein C levels correlate with purpura, digital infarction and death in meningococcemia.

Protein C is also altered in non-septic subjects following cardiopulmonary bypass (CPB). Total protein C, APC and protein S decrease during CPB. Following aortic unclamping (reperfusion at the end of CPB) protein C is further activated so that the proportion of remaining non-activated protein C is greatly decreased. A decrease of protein C during and after CPB increases the risk of thrombosis, disseminated intravascular coagulation (DIC), organ ischemia and inflammation intra- and post-operatively. Subjects who have less activated protein C generally have impaired recovery of cardiac function, consistent with the idea that lower levels of protein C increase the risk of microvascular thrombosis and myocardial ischemia. Aprotinin is a competitive inhibitor of APC, and is sometimes used in cardiac surgery and CPB. Aprotinin has been implicated as a cause of post-operative thrombotic complications after deep hypothermic circulatory arrest.

Septic and non-septic stimuli such as bacterial endotoxin and cardiopulmonary bypass (CPB), activate the coagulation system and trigger a systemic inflammatory response syndrome (SIRS). A decrease in protein C levels have been shown in subjects with septic shock (GRIFFIN J H. et al. (1982) Blood 60:261-264; TAYLOR F B. et al. (1987) J. Clin. Invest. 79:918-925; HESSELVIK J F. et al,.(1991) Thromb. Haemost. 65:126-129; FIJNVANDRAAT K. et al. (1995) Thromb. Haemost. 73(7):15-20), with severe infection (HESSELVIK J F. et al. (1991) Thromb. Haemost. 65:126-129) and after major surgery (BLAMEY S L. et al. (1985) Thromb. Haemost. 54:622-625). It has been suggested that this decrease is caused by a decrease in protein C transcription (SPEK CA. et al. J. Biological Chemistry (1995) 270(41):24216-21 at 24221). It has also been demonstrated that endothelial pathways required for protein C activation are impaired in severe menigococcal sepsis (FAUST S N. et al. New Eng. J. Med. (2001) 345:408-416). Low protein C levels in sepsis subjects are related to poor prognosis (YAN S B. and DHAINAUT J-F. Critical Care Medicine (2001) 29(7):S69-S74; FISHER C J. and YAN S B. Critical Care Medicine (2000) 28(9 Suppl):S49-S56; VERVLOET M G. et al. Semin Thromb Hemost. (1998) 24(1):33-44; LORENTE J A. et al. Chest (1993) 103(5):1536-42). Recombinant human activated protein C reduces mortality in subjects having severe sepsis or septic shock (BERNARD G R. et al. New Eng. J. Med. (2001) 344:699-709). Thus protein C appears to play an important beneficial role in the systemic inflammatory response syndrome.

The human protein C gene maps to chromosome 2q13-q14 and extends over 11 kb. A representative Homo sapiens protein C gene sequence is listed in GenBank under accession number AF378903. Three single nucleotide polymorphisms (SNPs) have been identified in the 5′ untranslated promoter region of the protein C gene and are characterized as −1654 C/T, −1641 A/G and −1476 A/T (according to the numbering scheme of FOSTER D C. et al. Proc Natl Acad Sci USA (1985) 82(14):4673-4677), or as −153C/T, −140A/G and +26A/T respectively by (MILLAR D S. et al. Hum. Genet. (2000) 106:646-653 at 651).

The genotype homozygous for −1654 C/−1641 G/−1476 T has been associated with reduced rates of transcription of the protein C gene as compared to the −1654 T/−1641 A/−1476 A homozygous genotype (SCOPES D. et al. Blood Coagul. Fibrinolysis (1995) 6(4):317-321). Subjects homozygous for the −1654 C/−1641 G/−1476 T genotype show a decrease of 22% in plasma protein C levels and protein C activity levels as compared to subjects homozygous for the −1654 T/−1641 A/−1476 A genotype (SPEK C A. et al. Arteriosclerosis, Thrombosis, and Vascular Biology (1995) 15:214-218). The −1654 C/−1641 G haplotype has been associated with lower protein C concentrations in both homozygotes and heterozygotes as compared to −1654 T/−1641 A (AIACH M. et al. Arterioscler Thromb Vasc Biol. (1999) 19(6):1573-1576).

The human endothelial protein C receptor (EPCR) gene sequence is located on chromosome 20 and maps to chromosome 20q11.2. A representative human EPCR gene sequence with promoter is listed in GenBank under accession number AF106202 (8167 bp). A number of polymorphisms have been observed in the EPCR gene (BIGUZZI E. et al. Thromb Haemost (2002) 87:1085-6 and FRANCHI F. et al. Br J Haematol (2001 ) 114:641-6). Furthermore, polymorphisms of EPCR are also described in (BIGUZZI E. et al. Thromb Haemost (2001) 86:945-8; GALLIGAN L. et al. Thromb Haemost (2002) 88:163-5; ZECCHINA G. et al. Br J Haematol (2002) 119:881-2; FRENCH J K. et al. Am Heart J (2003) 145:118-24; and VON DEPKA M. et al. Thromb Haemost (2001) 86:1360-2; and SAPOSNIK B. et al. Blood (Feb. 15, 2004) 103(4):1311-8.).

### SUMMARY OF THE INVENTION

This invention is based in part on the surprising discovery that the combination of predictive SNPs from the Protein C and Endothelial Protein C Receptor (EPCR) can be more accurate predictors of subject outcome than SNPs from either Protein C or EPCR alone.

This invention is also based in part on the surprising discovery of protein C SNPs previously uncharacterized with regards to an association with improved prognosis or subject outcome, in subjects with an inflammatory condition. Furthermore, various protein C polymorphisms are provided which are useful for subject screening, as an indication of subject outcome, or for prognosis for recovery from an inflammatory condition.

This invention is also based in part on the surprising discovery that EPCR SNPs previously uncharacterized with regards to an association with improved prognosis or subject outcome, in subjects with an inflammatory condition. Furthermore, various EPCR polymorphisms are provided which are useful for subject screening, as an indication of subject outcome, or for prognosis for recovery from an inflammatory condition.

This invention is also based in part on the identification the particular nucleotide (allele) at the site of a given SNP may be associated with a decreased likelihood of recovery from an inflammatory condition (‘risk genotype’) or an increased likelihood of recovery from an inflammatory condition (‘protective genotype’). Furthermore, this invention is in part based on the discovery that the risk genotype or allele may be predictive of increased responsiveness to the treatment of the inflammatory condition with the anti-inflammatory agent or the anti-coagulant agent. The anti-inflammatory agent or the anti-coagulant agent may be activated protein C. The inflammatory condition may be SIRS, sepsis or septic shock.

This invention is also based in part on the surprising discovery that both EPCR and protein C SNPs alone or in combination are useful in predicting the response a subject with an inflammatory condition will have to activated protein C treatment or treatment with another anti-inflammatory agent or anti-coagulant agent. Whereby the subjects having a risk genotype are more likely to benefit from and have an improved response to protein C treatment or treatment with another anti-inflammatory agent or anti-coagulant agent and subjects having a protective genotype are less likely to benefit from the same treatment.

In accordance with one aspect of the invention, methods are provided for obtaining a prognosis for a subject having, or at risk of developing, an inflammatory condition, the method including determining a genotype of said subject which includes one or more polymorphic sites in the subject's protein C sequence; EPCR sequence or a combination thereof, wherein said genotype is indicative of an ability of the subject to recover from the inflammatory condition. The method may further involve determination of the genotype for one or more polymorphic sites in the protein C sequence and one or more polymorphic sites in the EPCR sequence for the subject. The genotypes of the protein C sequence and EPCR sequence may be taken alone or in combination.

The protein C polymorphic site may be at position 4732 of SEQ ID. NO.: 1 or a polymorphic site linked thereto. The polymorphic site in linkage disequilibrium with position 4732 may be at position 4813, 6379, 6762, 7779, 8058, 8915 or 12228 of SEQ ID NO: 1. The polymorphic site in linkage disequilibrium with position 4732 may be a combination of two positions in SEQ ID NO: 1 selected from the following: 9198 and 5867; 9198 and 4800; 3220 and 5867; and 3220 and 4800 or 5′ (rs908787) and 3′ (rs777566, rs334135, rs777569, rs334142, rs334160, rs334159, rs334151, rs334146, rs777556, rs334144) to the protein C gene (SEQ ID NO:1). Such SNPs may be genotyped as an alternative to genotyping protein C SNP 4732 or other protein C SNPs within SEQ ID NO: 1 as an-indicator of improved prognosis or subject outcome, in subjects with an inflammatory condition or assessing a subjects risk genotype as described herein.

The EPCR polymorphic site may be at position 4054 of SEQ ID NO.: 2 or a polymorphic site linked thereto. The EPCR polymorphic site in linkage disequilibrium with position 4054 may be at position 2973, 3063, 3402, 4946, 5515 or 6196 of SEQ ID NO: 2 or 5′ (rs 2295887, rs1535466, rs033797, rs1033798, rs1033799, rs2295888, rs666210, rs1415771, rs945959) and 3′ (rs1051056, rs632688, rs633198, rs663550) to the EPCR gene (SEQ ID NO:2). These SNPs may be genotyped as an alternative to genotyping EPCR SNPs 4054 or 6196 or other EPCR SNPs within SEQ ID NO:2 as an indicator of improved prognosis or subject outcome, in subjects with an inflammatory condition or assessing a subjects risk genotype as described herein.

In accordance with another aspect of the invention, methods are provided for determining polymorphic sites from both the Protein C sequence and EPCR sequence in combination, wherein the polymorphic sites are at two or more of position 4732 of SEQ ID NO:1; or position 4054 of SEQ ID NO:2; or position 2418 of SEQ ID NO:1; or a polymorphic site in linkage disequilibrium thereto.

The polymorphic site in linkage disequilibrium with position 2418 may be at position 1386, 2583, or 3920 of SEQ ID NO: 1. The polymorphic site in linkage disequilibrium with position 2418 may be combination of two polymorphic sites, which sites may occur at any of the following combinations of positions in SEQ ID NO:1: 5867 and 2405; 5867 and 4919; 5867 and 4956; 5867 and 6187; 5867 and 12109; 4800 and 2405; 4800 and 4919; 4800 and 4956; 4800 and 6187.; 4800 and 12109.

The method may further include comparing the genotype determined with known genotypes which are known to be indicative of a prognosis for recovery from the subject's type of inflammatory condition; or another inflammatory condition.

The method may further include obtaining protein C sequence information or EPCR sequence information for the subject and may be determined using a nucleic acid sample from the subject. The method may further include obtaining the nucleic acid sample from the subject. Determining genotype may be accomplished using one or more of the following techniques: restriction fragment length analysis; sequencing; hybridization; oligonucleotide ligation assay; ligation rolling circle amplification; 5′ nuclease assay; polymerase proofreading methods; allele specific PCR; and reading sequence data.

The genotype of the subject may be indicative of a decreased ability to recover from the inflammatory condition (risk genotype). The subject may be critically ill and the genotype may be indicative of a prognosis of severe cardiovascular or respiratory dysfunction. The genotype may include at least one of the following single polymorphic nucleotides or combinations of polymorphic nucleotides at the indicated positions of SEQ ID NO: 1: 4732 C; 4813 A; 6379 G; 6762 A; 7779 C; 8058 T; 8915 T; 12228 T; 9198 C and 5867 A; 9198 C and 4800 G; 3220 A and 5867 A; and 3220 A and 4800 G; 1386 T; 2418 A; 2583 A; 3920 T; 5867 A and 2405 T; 5867 A and 4919 A; 5867 A and 4956 T; 5867 A and 6187 C; 5867 A and 12109 T; 4800, G and 2405 T; 4800 G and 4919 A, 4800 G and 4956 T; 4800 G and 6187 C; and 4800 G and 12109 T.

The genotype may include at least one of the following EPCR polymorphic nucleotides at the indicated positions of SEQ ID NO: 2: 6196 G; 5515 T; 4946 T; 4054 T; 3402 G; 3063 G; and 2973 C.

The genotype of the subject may be indicative of an increased ability to recover from the inflammatory condition. The subject may be critically ill and the genotype may be indicative of a prognosis of mild cardiovascular or respiratory dysfunction. The genotype may include at least one of the following single polymorphic nucleotides or combinations of polymorphic nucleotides at the indicated positions of SEQ ID NO: 1: 4732 T; 4813 G; 6379 A; 6762 G; 7779-; 8058 C; 8915 G; 12228 C; 9198 A and 5867 G; 9198 A and 4800 C; 3220 G and 5867 G; and 3220 G and 4800 C; 1386 C; 2418 G; 2583 T; 392Q C; 5867 G and 2405 C; 5867 G and 4919 G; 5867 G and 4956 C; 5867 G and 6187 T; 5867 G and 12109 C; 4800 C and 2405 C; 4800 C and 4919 G; 4800 C and 4956 C; 4800 C and 6187 T; and 4800 C and 12109 C. Alternatively, the genotype indicative of a prognosis of mild cardiovascular or respiratory dysfunction at position 7779 of SEQ ID NO:46 may be T.

The genotype may include at least one of the following EPCR polymorphic nucleotides at the indicated positions of SEQ ID NO: 2: 6196 C; 5515 C;4946 C; 4654 C; 3402 C, 3063 A; and 2973 T.

Where the genotype of the subject corresponding to the nucleotide in position 4732 of SEQ ID NO: 1, is cytosine (C), the prognosis may be indicative of a decreased likelihood of recovery from an inflammatory condition or of severe cardiovascular or respiratory dysfunction in critically ill subjects.

Where the genotype of the subject corresponding to the nucleotide in position 4732 of SEQ ID NO: 1, is thymine (T) the prognosis may be indicative of a increased likelihood of recovery from an inflammatory condition or of less severe cardiovascular or respiratory dysfunction in critically ill subjects.

Where the genotype of the subject corresponding to the nucleotide in position 4054 of SEQ ID NO: 2, is T, the prognosis may be indicative of a decreased likelihood of recovery from an inflammatory condition or of severe cardiovascular or respiratory dysfunction in critically ill subjects.

Where the genotype of the subject corresponding to the nucleotide in position 4054 of SEQ ID NO: 2, is cytosine (C), the prognosis may be indicative of a increased likelihood of recovery from an inflammatory condition or of less severe cardiovascular or respiratory dysfunction in critically ill subjects.

In accordance with another aspect of the invention, methods are provided for combining the protein C and EPCR polymorphic site genotype information, whereby the subjects are grouped according to genotype to improve the predictive value for determining a subject's ability to recover from an inflammatory condition over using either a protein C or an EPCR SNP alone.

Group 1 subjects have no copies of the EPCR risk allele (4054T) and no copies of the protein C risk allele (4732 C), group 2 subjects have at least one copy of the EPCR risk allele (4054T) and at least one copy of the protein C risk allele (4732C). Group 3 subjects can have either at least one copy of the EPCR risk allele (4054T) and no copies of the 25 protein C risk allele (4732C) or they can have no copies of the EPCR risk allele (4054 T) and at least one copy of the protein C risk allele (4732C). Group 1 subjects are expected to have the best outcomes, group 2 subjects are expected to have the worst outcomes and group 3 subjects are expected to have intermediate outcomes, with regards to recovering from an inflammatory condition.

The inflammatory condition may be selected from the group including: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke: congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, bifluenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may be SIRS, sepsis or septic shock. The inflammatory condition may be severe cardiovascular or respiratory dysfunction.

In accordance with another aspect of the invention, methods are provided for identifying a polymorphism in a protein C sequence that correlates with prognosis of recovery from an inflammatory condition, the method including: obtaining protein C sequence or EPCR sequence information from a group of subjects having an inflammatory condition; identifying at least one polymorphic nucleotide position in the protein C sequence or EPCR sequence in the subjects; determining a genotypes at the polymorphic site for individual subjects in the group; determining recovery capabilities of individual subjects in the group from the inflammatory condition; and correlating the genotypes determined in with the recovery capabilities determined thereby identifying said protein C or EPCR polymorphisms that correlate with recovery.

In accordance with another aspect of the invention, a kit is provided for determining a genotype at a defined nucleotide position within a polymorphic site in a protein C sequence or an EPCR sequence in a subject to provide a prognosis of the subject's ability to recover from an inflammatory condition, the kit including: a restriction enzyme capable of distinguishing alternate nucleotides at the polymorphic site; or a labeled oligonucleotide having sufficient complementary to the polymorphic site so as to be capable of hybridizing distinctively to said alternate. The polymorphic sites may be at one or more of positions 4732 of SEQ ID NO:1, 4054 of SEQ ID NO:2, or a polymorphic site in linkage disequilibrium thereto. The kit may be suitable for determining genotype at one or more nucleotide positions within each of the protein C sequence or the EPCR sequence, wherein the polymorphic sites are at one or more of positions 4732 of SEQ ID NO:1, 4054 of SEQ ID NO:2; 2418 of SEQ ID NO:1; or a polymorphic site in linkage disequilibrium thereto. The kit may further include an oligonucleotide or a set of oligonucleotides operable to amplify a region including the polymorphic site. The kit may further include a polymerization agent and may also include instructions for using the kit to determine genotype. The kit may also include one or more of the following: a package; instructions for using the kit to determine genotype; reagents such a buffers, nucleotides and enzymes. A kit as described herein may contain any combination of the following: a restriction enzyme capable of distinguishing alternate nucleotides at a protein C and/or EPCR polymorphic site; and/or a labeled oligonucleotide having sufficient complementary to the protein C and/or EPCR polymorphic site and capable of distinguishing said alternate nucleotides; and/or an oligonucleotide or a set of oligonucleotides suitable for amplifying a region including the protein C and/or EPCR polymorphic site. The kit may also include one or more of the following: a package; instructions for using the kit to determine genotype; reagents such a buffers, nucleotides and enzymes; and/or containers. The kit comprising a restriction enzyme may also comprise an oligonucleotide or a set of oligonucleotides suitable to amplify a region surrounding the polymorphic site, a polymerization agent and instructions for using the kit to determine genotype.

In accordance with another aspect of the invention, oligonucleotides are provided that may be used in the identification of protein C and/or EPCR polymorphisms in accordance with the methods described herein, the oligonucleotides are characterized in that the oligonucleotides hybridize under normal hybridization conditions with a region of one of sequences identified by SEQ ID NO:1, SEQ ID NO:2, etc, or their complements.

The determining of a genotype may be accomplished by any technique known in the art, including but not limited to one or more of: restriction fragment length analysis; sequencing; hybridization; oligonucleotide ligation assay; ligation rolling circle amplification; 5′ nuclease assay; polymerase proofreading methods; allele specific PCR; matrix assisted laser desorption ionization time of flight MALDI-TOF mass spectroscopy micro-sequencing assay; gene chip hybridization assays; and reading sequence data.

In accordance with another aspect of the invention, methods are provided for selecting a group of subjects for determining the efficacy of a candidate drug known or suspected of being useful for the treatment of ant inflammatory condition, the method may include determining a genotype at one or more polymorphic sites in the protein C sequence or EPCR sequence for each subject, wherein the genotype is indicative of the subject's ability to recover from the inflammatory condition and sorting subjects based on their genotype. The method may further include administering the candidate drug to the subjects or a subset of subjects and determining each subject's ability to recover from the inflammatory condition. The method may further include comparing subject response to the candidate drug based on genotype of the subject.

In accordance with another aspect of the invention, methods are provided for treating an inflammatory condition in a mammal in need thereof, the method including administering to the mammal an anti-inflammatory agent or an anti-coagulant agent, wherein the mammal has a protein C sequence or EPCR sequence risk genotype.

In accordance with another aspect of the invention, methods are provided for treating an inflammatory condition in a mammal in need thereof, the method including selecting a mammal having a risk genotype in their protein C sequence or EPCR sequence and administering to the mammal an anti-inflammatory agent or an anti-coagulant agent.

In accordance with another aspect of the invention, methods are provided for treating an inflammatory condition in a subject in need thereof, the method including administering to the subject an anti-inflammatory agent or an anti-coagulant agent, wherein said subject has a protein C sequence or EPCR sequence risk genotype.

In accordance with another aspect of the invention, methods are provided for treating an inflammatory condition in a subject in need thereof, the method including: selecting a subject having a risk genotype in their protein C sequence or EPCR sequence; and administering to said subject an anti-inflammatory agent or an anti-coagulant agent.

In accordance with another aspect of the invention, methods are provided for treating a subject with an inflammatory condition by administering an anti-inflammatory agent or an anti-coagulant agent, the method including administering the anti-inflammatory agent or the anti-coagulant agent to subjects that have a risk genotype in their-protein C sequence or EPCR sequence, wherein the risk genotype is predictive of increased responsiveness to the treatment of the inflammatory condition with the anti-inflammatory agent or the anti-coagulant agent.

In accordance with another aspect of the invention, methods are provided for identifying a subject with increased responsiveness to treatment of an inflammatory condition with an anti-inflammatory agent or an anti-coagulant agent, including the step of screening a population of subjects to identify those subjects that have a risk genotype in their protein C sequence or EPCR sequence, wherein the identification of a subject with a risk genotype in their protein C sequence or EPCR sequence is predictive of increased responsiveness to the treatment of the inflammatory condition with the anti-inflammatory agent or the anti-coagulant agent.

In accordance with another aspect of the invention, methods are provided for subject screening, comprising the steps of (a) obtaining protein C and/or EPCR sequence information from a subject, and (b) determining the identity of one or more polymorphisms in the sequence, wherein the one or more polymorphisms may be indicative of the ability of a subject to recover from an inflammatory condition.

In accordance with another aspect of the invention methods are provided for subject screening whereby the method includes the steps of (a) selecting a subject based on risk of developing an inflammatory condition or having an inflammatory condition, (b) obtaining protein C and/or EPCR sequence information from the subject and (c) detecting the identity of one or more polymorphisms in the protein C sequence and/or EPCR sequence, wherein the polymorphism is indicative of the ability of a subject to recover from an inflammatory condition.

In accordance with another aspect of the invention, methods are provided for selecting a group of subjects to determine the efficacy of a candidate drug known or suspected of being useful for the treatment of an inflammatory condition, the method including determining a genotype for one or more polymorphic sites in the protein C sequence and/or EPCR sequence for each subject, wherein said genotype is indicative of the subject's ability to recover from the inflammatory condition and sorting subjects based on their genotype. The method may also include administering the candidate drug to the subjects or a subset of subjects and determining each subject's ability to recover from the inflammatory condition. The method may also include the additional step of comparing subject response to the candidate drug based on genotype of the subject. Response to the candidate drug may be decided by determining each subject's ability to recover from the inflammatory condition.

In accordance with another aspect of the invention, methods are provided for selecting a subject for the treatment of an inflammatory condition with an anti-inflammatory agent or an anti-coagulant agent, including the step of identifying a subject having a risk genotype in their protein C sequence or EPCR sequence, wherein the identification of a subject with the risk genotype is predictive of increased responsiveness to the treatment of the inflammatory condition with the anti-inflammatory agent or the anti-coagulant agent.

In accordance with another aspect of the invention, methods are provided for treating an inflammatory condition in a subject, the method including administering an anti-inflammatory agent or an anti-coagulant agent to the subject, wherein said subject has a risk genotype in their protein C sequence or EPCR sequence.

In accordance with another aspect of the invention, methods are provided for treating an inflammatory condition in a subject, the method including: identifying a subject having a risk genotype in their protein C sequence or EPCR sequence; and administering an anti-inflammatory agent or an anti-coagulant agent to the subject.

In accordance with another aspect of the invention, the use of an anti-inflammatory agent or an anti-coagulant in the manufacture of a medicament for the treatment of an inflammatory condition is provided, wherein the subjects treated have a risk genotype in their protein C sequence or EPCR sequence.

In accordance with another aspect of the invention, the use of an anti-inflammatory agent or an anti-coagulant in the manufacture of a medicament for the treatment of an inflammatory condition in a subset of subjects is provided, wherein the sub set of subjects have a risk genotype in their protein C sequence or EPCR sequence.

In accordance with another aspect of the invention, a method of treating an inflammatory condition in a subject in need thereof is provided, the method including:(a) selecting a subject having a risk genotype in their protein C sequence or EPCR sequence; and (b) administering to said subject an anti-inflammatory agent or an anti-coagulant agent. Step (a) may be performed before or contemporaneously with step (b). Alternatively, step (a) may be performed subsequent to step (b) with a view to ceasing continued treatment with the anti-inflammatory agent or anti-coagulant agent if it is clear that the patient has a non-risk genotype and/or is not responding to the therapy.

The method may further include determining the subject's APACHE II score as an assessment of subject risk. The method or use may further include determining the number of organ system failures for the subject as an assessment of subject risk. The subject's APACHE II score may be indicative of an increased risk when ≧25. 2 or more organ system failures may be indicative of increased subject risk.

The risk genotype may be located at a polymorphic site at one or more of the following positions: 4732 of SEQ ID NO:1; 4054 of SEQ ID NO:2; or a polymorphic site in linkage disequilibrium thereto. The risk genotype may be located at a polymorphic site corresponding to position 2418 of SEQ ID NO:1 or a polymorphic site in linkage disequilibrium thereto. The risk genotypes from the Protein C sequence and EPCR sequence may be located at polymorphic sites at one or more of the following positions: 4732 of SEQ ID NO:1; 4054 of SEQ ID NO:2; 2418 of SEQ ID NO:1; or a polymorphic site in linkage disequilibrium thereto. The risk genotype may be located at a polymorphic site in linkage disequilibrium with position 4732 is at position 4813, 6379, 6762, 7779, 8058, 8915 or 12228 of SEQ ID NO: 1. The risk genotype may be located at a polymorphic site in linkage disequilibrium with position 4054 is at position 2973, 3063, 3402, 4946, 5515 or 6196 of SEQ ID NO: 2. The risk genotype may be located at a polymorphic site in linkage disequilibrium with position 2418 is at position 1386, 2583 or 3920 in SEQ ID NO: 1. The risk genotype may be located at a polymorphic site in linkage disequilibrium with position 4732 or a combination of two Protein C polymorphic sites, the combination being selected from the group of positions in SEQ ID NO: 1 including: 9198 and 5867; 9198 and 4800; 3220 and 5867; and 3220 and 4800. The risk genotype in linkage disequilibrium with position 2418 may be a combination of two Protein C polymorphic sites, the combination being selected from the group of positions in SEQ ID NO: 1 including: 5867 and 2405; 5867 and 4919; 5867 and 4956; 5867 and 6187; 5867 and 12109; 4800 and 2405; 4800 and 4919; 4800 and 4956; 4800 and 6187; and 4800 and 12109.

The risk genotype may selected from polymorphic sites and combined polymorphic sites in SEQ ID NO: 1 including: 4732 C; 4813 A; 6379 G; 6762 A; 7779 C; 8058 T; 8915 T; 12228 T; 9198 C and 5867 A; 9198 C and 4800 G; 3220 A and 5867 A; and 3220 A and 4800 G; 1386 T; 2418 A; 2583 A; 3920 T; 5867 A and 2405 T; 5867 A and 4919 A; 5867 A and 4956 T; 5867 A and 6187 C; 5867 A and 12109 T; 4800 G and 2405 T; 4800 G and 4919 A; 4800 G and 4956 T; 4800 G and 6187 C; and 4800 G and 12109 T.

The risk genotype may be selected from the group of EPCR polymorphic sites in SEQ ID NO: 2 including: 6196 G; 5515.T; 4946 T; 4054 T; 3402 G; 3063 G; and 2973 C.

The risk genotype may be indicative of an increased risk of poor outcome from an inflammatory condition. The method or use may further include preferentially selecting a subject with increased risk of poor outcome from an inflammatory condition for administration the anti-inflammatory agent or the anti-coagulant agent.

The protective genotype or decreased risk genotype may be selected from the group of protein C single polymorphic sites and combined polymorphic sites in SEQ ID NO: 1 including: 4732 T; 4813 G; 6379 A; 6762 G; 7779 -; 8058 C; 8915 G; 12228 C; 9198 A and 5867 G; 9198 A and 4800 C; 3220 G and 5867 G; and 3220 G and 4800 C; 1386 C; 2418 G; 2583 T; 3920 C; 5867 G and 2405 C; 5867 G and 4919 G; 5867 G and 4956 C; 5867 G and 6187 T; 5867 G and 12109 C; 4800 C and 2405 C; 4800 C and 4919 G; 4800 C and 4956 C; 4800 C and 6187 T; and 4800 C and 12109 C. The protective genotype or decreased risk genotype may selected from the group of EPCR polymorphic sites in SEQ ID NO: 2 including: 6196 C; 5515 C; 4946 C; 4054 C; 3402 C; 3063 A; and 2973 T. Alternatively, the protective genotype at position 7779 of SEQ ID NO:46 may be T.

The genotype of the subject may be indicative of a decreased risk of poor outcome from an inflammatory condition and the subject having a decreased risk of poor outcome from an inflammatory condition may be preferentially not selected for administration the anti-inflammatory agent or the anti-coagulant agent. The anti-inflammatory agent or the anti-coagulant agent may be selected from any one or more of the following: activated protein C; tissue factor pathway inhibitors; platelet activating factor hydrolase; PAF-AH enzyme analogues; antibody to tumor necrosis factor alpha; soluble tumor necrosis factor receptor-immunoglobulin G1; procysteine; elastase inhibitor; human recombinant interleukin 1 receptor antagonists; and antibodies, inhibitors and antagonists to endotoxin, tumour necrosis factor receptor, interleukin-6, high mobility group box, tissue plasminogen activator, bradykinin, CD-14 and interleukin-10. The anti-inflammatory agent or the anti-coagulant agent may be activated protein C or drotecogin alfa activated.

In accordance with another aspect of the invention, methods are provided for treatment of an inflammatory condition in an eligible subject by administering a treatment option, such as a therapeutic agent, after first determining if a subject is an eligible subject on the basis of the genetic sequence information or genotype information disclosed herein. Where the method of treatment of an inflammatory condition in an eligible subject may comprise the following: a) determining if a subject is an eligible subject on the basis of the presence or absence of one or more risk genotypes in the protein C sequence and/or EPCR sequence; and b) administering a therapeutic agent to the eligible subject. More specifically, the method of treatment of an inflammatory condition in an eligible subject may comprise: a) determining if a subject is an eligible subject on the basis of the presence or absence of one or more risk genotypes in the protein C sequence and/or EPCR sequence; and b) administering a therapeutic agent selected from among activated protein C (e.g. XIGRIS™ —drotecogin alfa-recombinant human activated protein C (Eli Lilly)), tissue factor pathway inhibitors (e.g. TIFACOGIN™—alpha (Chiron) and the like), platelet activating factor hydrolase (e.g. PAFase™ (ICOS) and other PAF-AH enzyme analogues), antibody to tumor necrosis factor-alpha (e.g. SEGARD™—afelimomab (Abbott)), or other anti-inflammatory therapeutic agent, to the eligible subject. Furthermore, the therapeutic agent may be activated protein C and/or a derivative thereof (including glycosylation mutants), alone or in combination or in combination with other therapeutic agents as described herein. An improved response to a therapeutic agent may include an improvement subsequent to administration of the therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR<1.5], renal and/or hepatic).

In accordance with another aspect of the invention, methods are provided for treatment of an inflammatory condition in an eligible subject comprising administering a therapeutic agent to an eligible subject. The eligible subject may be a subject having one or more of the polymorphisms in protein C and/or EPCR that are associated with decreased likelihood of recovery from an inflammatory condition, as disclosed herein or as later discovered. Treatment options, may include: activated protein C (e.g. XIGRIS™ drotecogin alfa-recombinant human activated protein C (Eli Lilly)), tissue factor pathway inhibitors (e.g. TIFACOGIN™ alpha (Chiron) and the like), platelet activating factor hydrolase (e.g. PAFase™ (ICOS) and other PAF-AH enzyme analogues), antibody to tumor necrosis factor-alpha (e.g. SEGARD™ afelimomab (Abbott)), soluble tumor necrosis factor receptor-immuoglobulin G1 (Roche), procysteine, elastase inhibitor, human recombinant interleukin 1 receptor antagonist (IL-1 RA), and antibodies, inhibitors and antagonists to: an endotoxin (i.e. lipopolysaccharide, LPS, lipotechoic acid and the like, e.g. E-5531 (Eisai)), tumour necrosis factor receptor, IL-6, high-mobility group box 1 (HMGB-1 or HMG-1), tissue plasminogen activator, bradykinin, CD-14, and/or IL-10. Those skilled in the art are familiar with the dosage and administration of these and other treatment options. To determine a subject's eligibility, the presence or absence of polymorphisms in the protein C sequence and/or EPCR sequence, may be determined as described herein.

Activated protein C (e.g. XIGRIS™ drotecogin alfa-recombinant human activated protein C (Eli Lilly)), tissue factor pathway inhibitors (e.g. TIFACOGIN™ alpha (Chiron) and the like), platelet activating factor hydrolase (e.g. PAFase™ (ICOS) and other PAF-AH enzyme analogues), antibody to tumor necrosis factor-alpha (e.g. SEGARD™ afelimomab (Abbott)), or other anti-inflammatory therapeutic agent, may be useful in the manufacture of a medicament for the, therapeutic treatment of an inflammatory condition in a subject having one or more of the polymorphisms in protein C and/or EPCR that are associated with decreased likelihood of recovery from an inflammatory condition. Furthermore these therapeutic agents may be useful in the preparation of an anti-sepsis agent in ready-to-use drug form for treating or preventing sepsis in a subject having one or more of the polymorphisms in protein C and/or EPCR that are associated with decreased likelihood of recovery from an inflammatory condition.

In accordance with another aspect of the invention, oligonucleotides are provided that may be used in the identification of protein C and/or EPCR polymorphisms in accordance with the methods described herein, the oligonucleotides are characterized in that the oligonucleotides hybridize under normal hybridization conditions with a region of one of sequences identified by SEQ ID NO:1, SEQ ID NO:2, etc. or their complements.

In accordance with another aspect of the invention, an oligonucleotide primer is provided comprising a portion of SEQ ID NO:1, SEQ ID NO:2 or their complements, wherein said primer is twelve to fifty-four nucleotides in length and wherein the primer specifically hybridizes to a region of SEQ ID NO:1, SEQ ID NO:2 or their complements and is capable of identifying protein C and/or EPCR sequence polymorphisms described herein. Alternatively, the primers may be between sixteen to twenty-four nucleotides in length.

In accordance with another aspect of the invention, oligonucleotide are provided of about 10 to about 400 nucleotides that hybridizes specifically to a sequence contained in a human target sequence including of SEQ ID NO:1, a complementary sequence of the target sequence or RNA equivalent of the target sequence and wherein the oligonucleotide is operable in determining a polymorphism genotype at position 4732, 4813, 6379, 6762, 7779, 8058, 8915, 12228, 9198, 5867, 4800, 3220, 1386, 2418, 2583, 3920, 2405, 4919, 4956, 6187 or 12109 of SEQ ID NO:1.

In accordance with another aspect of the invention, oligonucleotide are provided of about 10 to about 400 nucleotides that hybridizes specifically to a sequence contained in a human target sequence including of SEQ ID NO:2, a complementary sequence of the target sequence or RNA equivalent of the target sequence and wherein the oligonucleotide is operable in determining a polymorphism genotype at position 6196, 5515, 4946, 4054, 3402, 3063 or 2973 of SEQ ID NO:2.

In accordance with another aspect of the invention, oligonucleotide are provided of about 10 to about 400 nucleotides that hybridizes specifically to a sequence contained in a human target sequence including of SEQ ID NO:1, a complementary sequence of the target sequence or RNA equivalent of the target sequence and wherein said hybridization is operable in determining a polymorphism genotype at position 4732, 4813, 6379, 6762, 7779, 8058, 8915, 12228, 9198, 5867, 4800, 3220, 1386, 2418, 2583, 3920, 2405, 4919, 4956, 6187 or 12109 of SEQ ID NO:1.

In accordance with another aspect of the invention, oligonucleotide are provided of about 10 to about 400 nucleotides that hybridizes specifically to a sequence contained in a human target sequence including of SEQ ID NO:2, a complementary sequence of the target sequence or RNA equivalent of the target sequence and wherein said hybridization is operable in determining a polymorphism genotype at position 6196, 5515, 4946, 4054, 3402, 3063 or 2973 of SEQ ID NO:2.

In accordance with another aspect of the invention, oligonucleotide probes are provided which may be selected from the group including: (a) a probe that hybridizes under high stringency conditions to a nucleic acid molecule including SEQ ID NO:1 having a C at position 4732 but not to a nucleic acid molecule including SEQ ID NO:1 having a T at position 4732; (b) a probe that hybridizes under high stringency conditions to a nucleic acid molecule including SEQ ID NO: 1 having a T at position 4732 but not to a nucleic acid molecule including SEQ ID NO: 1 having a C at position 4732; (c) a probe that hybridizes under high stringency conditions to a nucleic acid molecule including SEQ ID NO:1 having a A at position 4813 but not to a nucleic acid molecule including SEQ ID NO:1 having a G at position 4813; (d) a probe that hybridizes under high stringency conditions to a nucleic acid molecule including SEQ ID NO:1 having a G at position 4813 but not to a nucleic acid molecule including SEQ ID NO:1 having a A at position 4813; (e) a probe that hybridizes under high stringency conditions to a nucleic acid molecule including SEQ ID NO:1 having a A at position 6379 but not to a nucleic acid molecule including SEQ ID NO:1 having a G at position 6379; (f) a probe that hybridizes under high stringency conditions to a nucleic acid molecule including SEQ ID NO:1 having a G at position 6379 but not to a nucleic acid molecule including SEQ ID NO:1 having a A at position 6379; (g) a probe that hybridizes under high stringency conditions to a nucleic acid molecule including SEQ ID NO:1 having a A at position 6762 but not to a nucleic acid molecule including SEQ ID NO:1 having a G at position 6762; (h) a probe that hybridizes under high stringency conditions to a nucleic acid molecule including SEQ ID NO:1 having a G at position 6762 but not to a nucleic acid molecule including SEQ ID NO:1 having a A at position 6762; (i) a probe that hybridizes under high stringency conditions to a nucleic acid molecule including SEQ ID NO:1 having a C at position 7779 but not to a nucleic acid molecule including SEQ ID NO:46 having a T at position 7779; (j) a probe that hybridizes under high stringency conditions to a nucleic acid molecule including SEQ ID NO:46 having a T at position 7779 but not to a nucleic acid molecule including SEQ ED NO:1 having a C at position 7779; (k) a probe that hybridizes under high stringency conditions to a nucleic acid molecule including SEQ ID NO:1 having a T at position 8058 but not to a nucleic acid molecule including SEQ ID NO:1 having a C at position 8058; (l) a probe that hybridizes under high stringency conditions to a nucleic acid molecule including SEQ ID NO:1 having a C at position 8058 but not to a nucleic acid molecule including SEQ ID NO:1 having a T at position 8058; (m) a probe that hybridizes under high stringency conditions to a nucleic acid molecule including SEQ ID NO:1 having a T at position 8915 but not to a nucleic acid molecule including SEQ ID NO:1 having a G at position 8915; (n) a probe that hybridizes under high stringency conditions to a nucleic acid molecule including SEQ ID NO:1 having a G at position 8915 but not to a nucleic acid molecule including SEQ ID NO:1 having a T at position 8915; (o) a probe that hybridizes under high stringency conditions to a nucleic acid molecule including SEQ ID NO:1 having a T at position 12228 but not to a nucleic acid molecule including SEQ ID NO:1 having a C at position 12228; (p) a probe that hybridizes under high stringency conditions to a nucleic acid molecule including SEQ ID NO:1 having a C at position 12228 but not to a nucleic acid molecule including SEQ ID NO:1 having a T at position 12228; (q) a probe that hybridizes under high stringency conditions to a nucleic acid molecule including SEQ ID NO:1 having a A at position 5867 but not to a nucleic acid molecule including SEQ ID NO:1 having a G at position 5867; (r) a probe that hybridizes under high stringency conditions to a nucleic acid molecule including SEQ ID NO:1 having a G at position 5867 but not to a nucleic acid molecule including SEQ ID NO:1 having a A at position 5867; (s) a probe that hybridizes under high stringency conditions to a nucleic acid molecule including SEQ ID NO:1 having a C at position 9198 but not to a nucleic acid molecule including SEQ ID NO:1 having a A at position 9198;(t) a probe that hybridizes under high stringency conditions to a nucleic acid molecule including SEQ ID NO:1 having a A at position 9198 but not to a nucleic acid molecule including SEQ ID NO:1 having a C at position 9198; (u) a probe that hybridizes under high stringency conditions to a nucleic acid molecule including SEQ ID NO:1 having a G at position 4800 but not to a nucleic acid molecule including SEQ ID NO:1 having a C at position 4800; (v) a probe that hybridizes under high stringency conditions to a nucleic acid molecule including SEQ ID NO:1 having a C at position 4800 but not to a nucleic acid molecule including SEQ ID NO:1 having a G at position 4800; (w) a probe that hybridizes under high stringency conditions to a nucleic acid molecule including SEQ ID NO:1 having a A at position 3220 but not to a nucleic acid molecule including SEQ ID NO:1 having a G at position 3220; (x) a probe that hybridizes under high stringency conditions to a nucleic acid molecule including SEQ ID NO:1 having a G at position 3220 but not to a nucleic acid molecule including SEQ ID NO:1 having a A at position 3220; (y) a probe that hybridizes under high stringency conditions to a nucleic acid molecule including SEQ ID NO:1 having a A at position 2418 but not to a nucleic acid molecule including SEQ ID NO:1 having a G at position 2418; and (z) a probe that hybridizes under high stringency conditions to a nucleic acid molecule including SEQ ID NO:1 having a G at position 2418 but not to a nucleic acid molecule including SEQ ID NO:1 having a A at position 2418.

Alternatively a probe may be selected that hybridizes under high stringency conditions to a nucleic acid molecule including SEQ ID NO:1 having a T at position 1386 but not to a nucleic acid molecule including SEQ ID NO:1 having a C at position 1386; a probe that hybridizes under high stringency conditions to a nucleic acid molecule including SEQ ID NO:1 having a C at position 1386 but not to a nucleic acid molecule including SEQ ID NO:1 having a T at position 1386; a probe that hybridizes under high stringency conditions to a nucleic acid molecule including SEQ ID NO:1 having a A at position 2583 but not to a nucleic acid molecule including SEQ ID NO:1 having a T at position 2583; a probe that hybridizes under high stringency conditions to a nucleic acid molecule including SEQ ID NO:1 having a T at position 2583 but not to a nucleic acid molecule including SEQ ID NO:1 having a A at position 2583; a probe that hybridizes under high stringency conditions to a nucleic acid molecule including SEQ ID NO:1 having a T at position 3920 but not to a nucleic acid molecule including SEQ ID NO:1 having a C at position 3920; a probe that hybridizes under high stringency conditions to a nucleic acid molecule including SEQ ID NO:1 having a C at position 3920 but not to a nucleic acid molecule including SEQ ID NO:1 having a T at position 3920; a probe that hybridizes under high stringency conditions to a nucleic acid molecule including SEQ ID NO:1 having a T at position 2405 but not to a nucleic acid molecule including SEQ ID NO:1 having a C at position 2405; a probe that hybridizes under high stringency conditions to a nucleic acid molecule including SEQ ID NO:1 having a C at position 2405 but not to a nucleic acid molecule including SEQ ID NO:1 having a T at position 2405; a probe that hybridizes under high stringency conditions to a nucleic acid molecule including SEQ ID NO:1 having a A at position 4919 but not to a nucleic acid molecule including SEQ ID NO:1 having a G at position 4919; a probe that hybridizes under high stringency conditions to a nucleic acid molecule including SEQ ID NO:1 having a G at position 4919 but not to a nucleic acid molecule including SEQ ID NO:1 having a A at position 4919; a probe that hybridizes under high stringency conditions to a nucleic acid molecule including SEQ ID NO:1 having a T at position 4956 but not to a nucleic acid molecule including SEQ ID NO:1 having a C at position 4956; a probe that hybridizes under high stringency conditions to a nucleic acid molecule including SEQ ED NO:1 having a C at position 4956 but not to a nucleic acid molecule including SEQ ID NO:1 having a T at position 4956; a probe that hybridizes under high stringency conditions to a nucleic acid molecule including SEQ ID NO:1 having a C at position 6187 but not to a nucleic acid molecule including SEQ ID NO:1 having a T at position 6187; a probe that hybridizes under high stringency conditions to a nucleic acid molecule including SEQ ID NO:1 having a T at position 6187 but not to a nucleic acid molecule including SEQ ID NO:1 having a C at position 6187; a probe that hybridizes under high stringency conditions to a nucleic acid molecule including SEQ ID NO:1 having a T at position 12109 but not to a nucleic acid molecule including SEQ ID NO:1 having a C at position 12109; and a probe that hybridizes under high stringency conditions to a nucleic acid molecule including SEQ ID NO:1 having a C at position 12109 but not to a nucleic acid molecule including SEQ ID NO:1 having a T at position 12109.

In accordance with another aspect of the invention, oligonucleotide probes methods are provided which may be selected from the group including: (a) a probe that hybridizes under high stringency conditions to a nucleic acid molecule including SEQ ID NO:2 having a C at position 2973 but not to a nucleic acid molecule including SEQ ID NO:2 having a T at position 2973; (b) a probe that hybridizes under high stringency conditions to a nucleic acid molecule including SEQ ID NO:2 having a T at position 2973 but not to a nucleic acid molecule including SEQ ID NO:2 having a C at position 2973; (c) a probe that hybridizes under high stringency conditions to a nucleic acid molecule including SEQ ID NO:2 having a G at position 3063 but not to a nucleic acid molecule including SEQ ID NO:2 having a A at position 3063; (d) a probe that hybridizes under high stringency conditions to a nucleic acid molecule including SEQ ID NO:2 having a A at position 3063 but not to a nucleic acid molecule including SEQ ID NO:2 having a G at position 3063; (e) a probe that hybridizes under high stringency conditions to a nucleic acid molecule including SEQ ID NO:2 having a G at position 3402 but not to a nucleic acid molecule including SEQ ID NO:2 having a C at position 3402; (f) a probe that hybridizes under high stringency conditions to a nucleic acid molecule including SEQ ID NO:2 having a C at position 3402 but not to a nucleic acid molecule including SEQ ID NO:2 having a G at position 3402; (g) a probe that hybridizes under high stringency conditions to a nucleic acid molecule including SEQ ID NO:2 having a T at position 4054 but not to a nucleic acid molecule including SEQ ID NO:2 having a C at position 4054; (h) a probe that hybridizes under high stringency conditions to a nucleic acid molecule including SEQ ID NO:2 having a C at position 4054 but not to a nucleic acid molecule including SEQ ID NO:2 having a T at position 4054; (i) a probe that hybridizes under high stringency conditions to a nucleic acid molecule including SEQ ID NO:2 having a T at position 4946 but not to a nucleic acid molecule including SEQ ID NO:2 having a C at position 4946; (j) a probe that hybridizes under high stringency conditions to a nucleic acid molecule including SEQ ID NO:2 having a C at position 4946 but not to a nucleic acid molecule including SEQ DID NO:2 having a T at position 4946; (k) a probe that hybridizes under high stringency conditions to a nucleic acid molecule including SEQ ID NO:2 having a T at position 5515 but not to a nucleic acid molecule including SEQ ID NO:2 having a C at position 5515; (l) a probe that hybridizes under high stringency conditions to a nucleic acid molecule including SEQ ID NO:2 having a C at position 5515 but not to a nucleic acid molecule including SEQ ID NO:2 having a T at position 5515; (m) a probe that hybridizes under high stringency conditions to a nucleic acid molecule including SEQ ID NO:2 having a G at position 6196 but not to a nucleic acid molecule including SEQ ID NO:2 having a C at position 6196; and (n) a probe that hybridizes under high stringency conditions to a nucleic acid molecule including SEQ ID NO:2 having a C at position 6196 but not to a nucleic acid molecule including SEQ ID NO:2 having a G at position 6196.

In accordance with another aspect of the invention arrays of nucleic acid molecules attached to a solid support are provided, the array may include one or more oligonucleotides selected from the group including: (a) an oligonucleotide that will hybridize to a nucleic acid molecule including SEQ ID NO:1 having a C at position 4732 but not to a nucleic acid molecule including SEQ ID NO:1 having a T at position 4732; (b) an oligonucleotide that will hybridize to a nucleic acid molecule including SEQ ID NO:1 having a T at position 4732 but not to a nucleic acid molecule including SEQ ID NO:1 having a C at position 4732; (c) an oligonucleotide that will hybridize to a nucleic acid molecule including SEQ ID NO:1 having a A at position 4813 but not to a nucleic acid molecule including SEQ ID NO:1 having a G at position 4813; (d) an oligonucleotide that will hybridize to a nucleic acid molecule including SEQ ID NO:1 having a G at position 4813 but not to a nucleic acid molecule including SEQ ID NO:1 having a A at position 4813; (e) an oligonucleotide that will hybridize to a nucleic acid molecule including SEQ ID NO:1 having a A at position 6379 but not to a nucleic acid molecule including SEQ ID NO:1 having a G at position 6379; (f) an oligonucleotide that will hybridize to a nucleic acid molecule including SEQ ID NO:1 having a G at position 6379 but not to a nucleic acid molecule including SEQ ID NO:1 having a A at position 6379; (g) an oligonucleotide that will hybridize to a nucleic acid molecule including SEQ ID NO:1 having a A at position 6762 but not to a nucleic acid molecule including SEQ ID NO:1 having a G at position 6762; (h) an oligonucleotide that will hybridize to a nucleic acid molecule including SEQ ID NO:1 having a G at position 6762 but not to a nucleic acid molecule including SEQ ID NO:1 having a A at position 6762; (i) an oligonucleotide that will hybridize to a nucleic acid molecule including SEQ ID NO:1 having a C at position 7779 but not to a nucleic acid molecule including SEQ ID NO:46 having a T at position 7779; (j) an oligonucleotide that will hybridize to a nucleic acid molecule including SEQ ID NO:46 having a T at position 7779 but not to a nucleic acid molecule including SEQ ID NO:1 having a C at position 7779; (k) an oligonucleotide that will hybridize to a nucleic acid molecule including SEQ ID NO:1 having a T at position 8058 but not to a nucleic acid molecule including SEQ ID NO:1 having a C at position 8058; (l) an oligonucleotide that will hybridize to a nucleic acid molecule including SEQ ID NO:1 having a C at position 8058 but not to a nucleic acid molecule including SEQ ID NO:1 having a T at position 8058; (m) an oligonucleotide that will hybridize to a nucleic acid molecule including SEQ ID NO:1 having a T at position 8915 but not to a nucleic acid molecule including SEQ ID NO:1 having a G at position 8915; (n) an oligonucleotide that will hybridize to a nucleic acid molecule including SEQ ID NO:1 having a G at position 8915 but not to a nucleic acid molecule including SEQ ID NO:1 having a T at position 8915; (o) an oligonucleotide that will hybridize to a nucleic acid molecule including SEQ ID NO:1 having a T at position 12228 but not to a nucleic acid molecule including SEQ ID NO:1 having a C at position 12228; (p) an oligonucleotide that will hybridize to a nucleic acid molecule including SEQ ID NO:1 having a C at position 12228 but not to a nucleic acid molecule including SEQ ID NO:1 having a T at position 12228; (q) an oligonucleotide that will hybridize to a nucleic acid molecule including SEQ ID NO:1 having a A at position 5867 but not to a nucleic acid molecule including SEQ ID NO:1 having a G at position 5867; (r) an oligonucleotide that will hybridize to a nucleic acid molecule including SEQ ID NO:1 having a G at position 5867 but not to a nucleic acid molecule including SEQ ID NO:1 having a A at position 5867; (s) an oligonucleotide that will hybridize to a nucleic acid molecule including SEQ ID NO:1 having a C at position 9198 but not to a nucleic acid molecule including SEQ ID NO:1 having a A at position 9198; (t) an oligonucleotide that will hybridize to a nucleic acid molecule including SEQ ID NO:1 having a A at position 9198 but not to a nucleic acid molecule including SEQ ID NO:1 having a C at position 9198; (u) an oligonucleotide that will hybridize to a nucleic acid molecule including SEQ ID NO:1 having a G at position 4800 but not to a nucleic acid molecule including SEQ ID NO:1 having a C at position 4800; (v) an oligonucleotide that will hybridize to a nucleic acid molecule including SEQ ID NO:1 having a C at position 4800 but not to a nucleic acid molecule including SEQ ID NO:1 having a G at position 4800; (w) an oligonucleotide that will hybridize to a nucleic acid molecule including SEQ ID NO:1 having a A at position 3220 but not to a nucleic acid molecule including SEQ ID NO:1 having a G at position: 3220; (x) an oligonucleotide that will hybridize to a nucleic acid molecule including SEQ ID NO:1 having a G at position 3220 but not to a nucleic acid molecule including SEQ ID NO:1 having a A at position 3220; (y) an oligonucleotide that will hybridize to a nucleic acid molecule including SEQ ID NO: 1 having a A at position 2418 but not to a nucleic acid molecule including SEQ ID NO:1 having a G at position 2418; and (z) an oligonucleotide that will hybridize to a nucleic acid molecule including SEQ ID NO:1 having a G at position 2418 but not to a nucleic acid molecule including SEQ ID NO:1 having a A at position 2418.

Alternatively an oligonucleotide may be selected that will hybridize to a nucleic acid molecule including SEQ ID NO:1 having a T at position 1386 but not to a nucleic acid molecule including SEQ ID NO:1 having a C at position 1386; an oligonucleotide that will hybridize to a nucleic acid molecule including SEQ ID NO:1 having a C at position 1386 but not to a nucleic acid molecule including SEQ ID NO:1 having a T at position 1386; an oligonucleotide that will hybridize to a nucleic acid molecule including SEQ ID NO:1 having a A at position 2583 but not to a nucleic acid molecule including SEQ ID NO:1 having a T at position 2583; an oligonucleotide that will hybridize to a nucleic acid molecule including SEQ ID NO::1 having a T at position 2583 but not to a nucleic acid molecule including SEQ ID NO:1 having a A at position 2583; an oligonucleotide that will hybridize to a nucleic acid molecule including SEQ ID NO:1 having a T at position 3920 but not to a nucleic acid molecule including SEQ ID NO:1 having a C at position 3920; an oligonucleotide that will hybridize to a nucleic acid molecule including SEQ ID NO:1 having a C at position 3920 but not to a nucleic acid molecule including SEQ ID NO:1 having a T at position 3920; an oligonucleotide that will hybridize to a nucleic acid molecule including SEQ ID NO:1 having a T at position 2405 but not to a nucleic acid molecule including SEQ ID NO:1 having a C at position 2405; an oligonucleotide that will hybridize to a nucleic acid molecule including SEQ ID NO:1 having a C at position 2405 but not to a nucleic acid molecule including SEQ ID NO:1 having a T at position 2405; an oligonucleotide that will hybridize to a nucleic acid molecule including SEQ ID NO:1 having a A at position 4919 but not to a nucleic acid molecule including SEQ ID NO:1 having a G at position 4919; an oligonucleotide that will hybridize to a nucleic acid molecule including SEQ ID NO:1 having a G at position 4919 but not to a nucleic acid molecule including SEQ ID NO:1 having a A at position 4919; an oligonucleotide that will hybridize to a nucleic acid molecule including SEQ ID NO:1 having a T at position 4956 but not to a nucleic acid molecule including SEQ ID NO:1 having a C at position 4956; an oligonucleotide that will hybridize to a nucleic acid molecule including SEQ ID NO:1 having a C at position 4956 but not to a nucleic acid molecule including SEQ ID NO:1 having a T at position 4956; an oligonucleotide that will hybridize to a nucleic acid molecule including SEQ ID NO:1 having a C at position 6187 but not to a nucleic acid molecule including SEQ ID NO:1 having a T at position 6187; an oligonucleotide that will hybridize to a nucleic acid molecule including SEQ ID NO:1 having a T at position 6187 but not to a nucleic acid molecule including SEQ ID NO:1 having a C at position 6187; an oligonucleotide that will hybridize to a nucleic acid molecule including SEQ ID NO:1 having a T at position 12109 but not to a nucleic acid molecule including SEQ ID NO:1 having a C at position 12109; and an oligonucleotide that will hybridize to a nucleic acid molecule including SEQ ID NO:1 having a C at position 12109 but not to a nucleic acid molecule including SEQ ID NO:1 having a T at position 12109.

In accordance with another aspect of the invention arrays of nucleic acid molecules attached to a solid support are provided, the array may include one or more oligonucleotides selected from the group including: (a) an oligonucleotide that will hybridize to a nucleic acid molecule including SEQ ID NO:2 having a C at position 2973 but not to a nucleic acid molecule including SEQ ID NO:2 having a T at position 2973; (b) an oligonucleotide that will hybridize to a nucleic acid molecule including SEQ ID NO:2 having a T at position 2973 but not to a nucleic acid molecule including SEQ ID NO:2 having a C at position 2973; (c) an oligonucleotide that will hybridize to a nucleic acid molecule including SEQ ID NO:2 having a G at position 3063 but not to a nucleic acid molecule including SEQ ID NO:2 having a A at position 3063; (d) an oligonucleotide that will hybridize to a nucleic acid molecule including SEQ ID NO:2 having a A at position 3063 but not to a nucleic acid molecule including SEQ ID NO:2 having a G at position 3063; (e) an oligonucleotide that will hybridize to a nucleic acid molecule including SEQ ID NO:2 having a G at position 3402 but not to a nucleic acid molecule including SEQ ID NO:2 having a C at position 3402; (f) an oligonucleotide that will hybridize to a nucleic acid molecule including SEQ ID NO:2 having a C at position 3402 but not to a nucleic acid molecule including SEQ ID NO:2 having a G at position 3402; (g) an oligonucleotide that will hybridize to a nucleic acid molecule including SEQ ID NO:2 having a T at position 4054 but not to a nucleic acid molecule including SEQ ID NO:2 having a C at position 4054; (h) an oligonucleotide that will hybridize to a nucleic acid molecule including SEQ ID NO:2 having a C at position 4054 but not to a nucleic acid molecule including SEQ ID NO:2 having a T at position 4054; (i) an oligonucleotide that will hybridize to a nucleic acid molecule including SEQ ID NO:2 having a T at position 4946 but not to a nucleic acid molecule including SEQ ID NO:2 having a C at position 4946; (j) an oligonucleotide that will hybridize to a nucleic acid molecule including SEQ ID NO:2 having a C at position 4946 but not to a nucleic acid molecule including SEQ ID NO:2 having a T at position 4946; (k) an oligonucleotide that will hybridize to a nucleic acid molecule including SEQ D) NO:2 having a T at position 5515 but not to a nucleic acid molecule including SEQ ID NO:2 having a C at position 5515; (l) an oligonucleotide that will hybridize to a nucleic acid molecule including SEQ ID NO:2 having a C at position 5515 but not to a nucleic acid molecule including SEQ ID NO:2 having a T at position 5515; (m) an oligonucleotide that will hybridize to a nucleic acid molecule including SEQ ID NO:2 having a G at position 6196 but not to a nucleic acid molecule including SEQ ID NO:2 having a C at position 6196; and (n) an oligonucleotide that will hybridize to a nucleic acid molecule including SEQ ID NO:2 having a C at position 6196 but not to a nucleic acid molecule including SEQ ID NO:2 having a G at position 6196.

In accordance with another aspect of the invention arrays of nucleic acid molecules attached to a solid support are provided, the array may include one or more oligonucleotides selected from the group including: (a) an oligonucleotide that will hybridize to a nucleic acid molecule including SEQ ID NO:3 having a G at position 201 but not to a nucleic acid molecule represented by the same SEQ ID NO having a A at position 201; (b) an oligonucleotide that will hybridize to a nucleic acid molecule including SEQ ID NO:3 having a A at position 201 but not to a nucleic acid molecule represented by the same SEQ ID NO having a G at position 201; (c) an oligonucleotide that will hybridize to a nucleic acid molecule including SEQ ID NO:4, SEQ ID NO:10, SEQ ID NO:11, SEQ ID NO:14, SEQ ID NO:15, SEQ ID NO:18, SEQ ID NO:19, or SEQ ID NO:20 having a T at position 201 but not to a nucleic acid molecule represented by the same SEQ ID NO having a C at position 201; (d) an oligonucleotide that will hybridize to a nucleic acid molecule including SEQ ID NO:4, SEQ ID NO:10, SEQ ID NO:11, SEQ ID NO:14, SEQ ID NO:15, SEQ ID NO:18, SEQ ID NO:19, or SEQ ID NO:20 having a C at position 201 but not to a nucleic acid molecule represented by the same SEQ ID NO having a T at position 201; (e) an oligonucleotide that will hybridize to a nucleic acid molecule including SEQ ID NO:5, SEQ ID NO:17, or SEQ ID NO:21 having a C at position 201 but not to a nucleic acid molecule represented by the same SEQ ID NO having a G at position 201; (f) an oligonucleotide that will hybridize to a nucleic acid molecule including SEQ ID NO:5, SEQ ID NO:17, or SEQ ID NO:21 having a G at position 201 but not to a nucleic acid molecule represented by the same SEQ ID NO having a C at position 201; (g) an oligonucleotide that will hybridize to a nucleic acid molecule including SEQ ID NO:12 having a G at position 201 but not to a nucleic acid molecule including SEQ ID NO:12 having a T at position 201; (h)an oligonucleotide that will hybridize to a nucleic acid molecule including SEQ ID NO:12 having a T at position 201 but not to a nucleic acid molecule including SEQ ID NO:12 having a G at position 201; (i) an oligonucleotide that will hybridize to a nucleic acid molecule including SEQ ID NO:12 having a T at position 201 but not to a nucleic acid molecule including SEQ ID NO:12 having a G at position 201; and (j) an oligonucleotide that will hybridize to a nucleic acid molecule including SEQ ID NO:13 having a A at position 201 but not to a nucleic acid molecule including SEQ ID NO:13 having a C at position 201.

In accordance with another aspect of the invention arrays of nucleic acid molecules attached to a solid support are provided, the array may include one or more oligonucleotides selected from the group including: (a) an oligonucleotide that will hybridize to a nucleic acid molecule including SEQ ID NO:22, SEQ ID NO:23, or SEQ ID NO:41 having a C at position 15 but not to a nucleic acid molecule represented by the same SEQ ID NO having a G at position 15; (b) an oligonucleotide that will hybridize to a nucleic acid molecule including SEQ ID NO:22, SEQ ID NO:23, or SEQ ID NO:41 having a G at position 15 but not to a nucleic acid molecule represented by the same SEQ ID NO having a C at position 15; (c) an oligonucleotide that will hybridize to a nucleic 10 acid molecule including SEQ ID NO:24, SEQ ID NO:27, SEQ ID NO:28, SEQ ID NO:29, SEQ ID NO:31, SEQ ID NO:35, SEQ ID NO:36, SEQ ID NO:39, or SEQ ID NO:44 having a C at position 15 but not to a nucleic acid molecule represented by the same SEQ ID NO having a G at position 15; (d) an oligonucleotide that will hybridize to a nucleic acid molecule including SEQ ID NO:24, SEQ ID NO:27, SEQ ID NO:28, SEQ ID NO:29, SEQ ID NO:31, SEQ ID NO:35, SEQ ID NO:36, SEQ ID NO:39, or SEQ ID NO:44 having a G at position 15 but not to a nucleic acid molecule represented by the same SEQ ID NO having a C at position 15; (e) an oligonucleotide that will hybridize to a nucleic, acid molecule including SEQ ID NO:25 or SEQ ID NO:49 having a A at position 15 but not to a nucleic acid molecule represented by the same SEQ ID NO having a T at position 15; (f) an oligonucleotide that will hybridize to a nucleic acid molecule including SEQ ID NO:25 or SEQ ID NO:49 having a T at position 15 but not to a nucleic acid molecule represented by the same SEQ ID NO having a A at position 15; (g) an oligonucleotide that will hybridize to a nucleic acid molecule including SEQ ID NO:26, SEQ ID NO:33, SEQ ID NO:34, SEQ ID NO:38, or SEQ ID NO:40 having a A at position 15 but not to a nucleic acid molecule represented by the same SEQ ID NO having a G at position 15; (h) an oligonucleotide that will hybridize to a nucleic acid molecule including SEQ ID NO:26, SEQ ID NO:33, SEQ ID NO:34, SEQ ID NO:38, or SEQ ID NO:40 having a G at position 15 but not to a nucleic acid molecule represented by the same SEQ ID NO having a A at position 15; (i) an oligonucleotide that will hybridize to a nucleic acid molecule including SEQ ID NO:30, SEQ ID NO:32, SEQ ID NO:37, SEQ ID NO:42, or SEQ ID NO:43 having a C at position 15 but not to a nucleic acid molecule represented by the same SEQ ID NO having a A at position 15; and (j) an oligonucleotide that will hybridize to a nucleic acid molecule including SEQ ID NO:30, SEQ ID NO:32, SEQ ID NO:37, SEQ ID NO:42, or SEQ ID NO:43 having a A at position 15 but not to a nucleic acid molecule represented by the same SEQ ID NO having a C at position 15.

In accordance with another aspect of the invention an array of nucleic acid molecules attached to a solid support is provided, the array may include an oligonucleotide that will hybridize to a nucleic acid molecule including SEQ ID NO:1, wherein the nucleotide at position 4732 is C, under conditions in which the oligonucleotide will not substantially hybridize to a nucleic acid molecule including SEQ ID NO:1 wherein the nucleotide at position 4732 is T.

In accordance with another aspect of the invention an array of nucleic acid molecules attached to a solid support is provided, the array may include an oligonucleotide that will hybridize to a nucleic acid molecule including SEQ ID NO:1, wherein the nucleotide at position 4732 is T, under conditions in which the oligonucleotide will not substantially hybridize to a nucleic acid molecule including SEQ ID NO:1 wherein the nucleotide at position 4732 is C.

In accordance with another aspect of the invention an array of nucleic acid molecules attached to a solid support is provided, the array may include an oligonucleotide that will hybridize to a nucleic acid molecule including SEQ ID NO:2, wherein the nucleotide at position 4054 is T, under conditions in which the oligonucleotide will not substantially hybridize to a nucleic acid molecule including SEQ ID NO:2 wherein the nucleotide at position 4054 is C.

In accordance with another aspect of the invention an array of nucleic acid molecules attached to a solid support is provided, the array may include an oligonucleotide that will hybridize to a nucleic acid molecule including SEQ ID NO:1, wherein the nucleotide at position 4054 is C, under conditions in which the oligonucleotide will not substantially hybridize to a nucleic acid molecule including SEQ ID NO:1 wherein the nucleotide at position 4054 is T.

The oligonucleotide may further include one or more of the following: a detectable label; a quencher; a mobility modifier; a contiguous non-target sequence situated 5′ or 3′ to the target sequence or 5′ and 3′ to the target sequence. The oligonucleotides may be used in conjunction with the methods and kits described herein to determine the genotypes of protein C and EPCR SNPs.

In accordance with another aspect of the invention a computer readable medium is provided, wherein the medium may have a plurality of digitally encoded genotype correlations selected from the Protein C and EPCR genotype correlations in TABLE 1E, wherein each correlation of the plurality has a value representing an ability to recover from an inflammatory condition and an indication of responsiveness to treatment of an inflammatory condition with an anti-inflammatory agent or an anti-coagulant agent.

### BRIEF DESCRIPTION OF THE DRAWINGS

FIG. 1 shows haplotypes and haplotype clades of the protein C sequence.

FIG. 2 shows haplotypes and haplotype clades of the endothelial protein C receptor (EPCR) sequence.

FIG. 3A Acute lung injury of subjects with the EPCR 4054 T allele (TC and TT) as compared to CC genotype.

FIG. 3B Cardiovascular dysfunction of subjects with the EPCR 4054 T allele (TC and TT) as compared to CC genotype.

FIG. 4 shows a Kaplan-Meier curve of the survival by genotype of endothelial protein C receptor 4054 T/C in critically ill subjects with SIRS over 28 days.

FIG. 5A shows a Kaplan-Meier curve of the survival by genotype of protein C 4732 T/C in critically ill subjects with SIRS over 28 days.

FIG. 5B shows a Kaplan-Meier curve of the survival by genotype of protein C 4732 T/C in critically ill subjects with Sepsis over 28 days.

FIG. 6A shows a Kaplan-Meier curve of the survival of groups 1, 2 and 3 of the protein C 4732/EPCR 4054 haplotypes over 28 days in a Sepsis subgroup.

FIG. 6B shows a Kaplan-Meier curve of the survival of groups 1, 2 and 3 of the protein C 4732/EPCR 4054 haplotypes over 28 days in a Septic Shock subgroup.

FIG. 7 shows Kaplan-Meier survival curves of patients who were protein C 4732 CC/CT who were and who were not treated with XIGRIS™.

FIG. 8 shows Kaplan-Meier survival curves of patients who were protein C 4732 TT who were and were not treated with XIGRIS™.

FIG. 9 shows Kaplan-Meier survival curves of patients who were EPCR 4054TT who were and who were not treated with XIGRIS™.

FIG. 10 shows Kaplan-Meier survival curves of patients who were EPCR 4054CT who were and were not treated with XIGRIS™.

FIG. 11 shows Kaplan-Meier survival curves of patients who were EPCR 4054CC who were and were not treated with XIGRIS™.

FIG. 12 shows Kaplan-Meier survival curves of patients who were classified as patients who carried at least one copy of protein C 4732C and one copy of EPCR 4054 T (risk-risk) who were and who were not treated with XIGRIS™.

FIG. 13 shows Kaplan-Meier survival curves of patients who carried one copy of either protein C 4732C or EPCR 4054 (risk-protective) and the protective-protective groups combined into a group called “other” who were and were not treated with XIGRIS™.

FIG. 14 shows a Kaplan Meier survival curve over 28 days of Asian cohort patients who were protein C 4732 CC or CT, and protein C 4732 TT.

FIG. 15 shows an association between protein C 4732 T/C genotype and days alive and free of steroid support in critically ill Asians with SIRS (p=0.086).

FIG. 16 shows a Kaplan Meier survival curve over 28 days of patients who had protein C 4732 CC or CT, and protein C 4732 TT.

FIG. 17 shows an association between protein C 4732 T/C genotype and days alive and free of steroid support (p=0.020) and between genotype and days alive and free of coagulation (p=0.038) in critically ill Asians with sepsis.

### DETAILED DESCRIPTION OF THE INVENTION

### 1. DEFINITIONS

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G).

“Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′5′ phosphodiester linkages. As used herein “purine” is used to refer to the purine bases, A and G and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C.

A “polymorphic site” or “polymorphism site” or “polymorphism” or “single nucleotide polymorphism site” (SNP site) as used herein is the locus or position with in a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes.

A “risk genotype” as used herein refers to an allelic variant (genotype) at one or more polymorphic sites within the Protein C sequence or EPCR sequence described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Risk genotype may be an indication of an increased risk of not recovering from an inflammatory condition. Subjects having one copy (heterozygotes) or two copies (homozygotes) of the risk allele (for example 4732 CT or 4732 CC or alternatively 4054 TC or 4054 TT or a combination thereof) are considered to have the “risk genotype” even though the degree to which the subjects risk of not recovering from an inflammatory condition increases, may be greater for homozygotes over heterozygotes. Such “risk alleles” or “risk genotype” may be selected from the group of protein C single polymorphic sites and combined polymorphic sites in SEQ ID NO:1 consisting of:
4732 C;
4813 A;
6379 G;
6762 A;
7779 C;
8915 T;
12228 T;
9198 C and 5867 A;
9198 C and 4800 G;
3220 A and 5867 A; and
3220 A and 4800 G
or
1386 T;
2418 A;
2583 A;
3920 T;
5867 A and 2405 T;
5867 A and 4919 A;
5867 A and 4956 T;
5867 A and 6187 C;
5867 A and 12109 T;
4800 G and 2405 T;
4800 G and 4919 A;
4800 G and 4956 T;
4800 G and 6187 C; and
4800 G and 12109 T.

Or such “risk alleles” or “risk genotype” may be selected from the group of EPCR single polymorphic sites in SEQ ID NO: 2 consisting of:
6196 G;
5515 T;
4946 T;
4054 T;
3402 G;
3063 G; and
2973 C.

Furthermore, single and/or combined polymorphic sites of protein C and single and/or combined polymorphic sites of EPCR may be further combined (for example protein C 4732 with EPCR 4054) to increase the predictive value and to improve the determinative value in deciding whether to treat a subject or mammal with an anti-inflammatory agent or an anti-coagulant agent.

A “clade” is a group of haplotypes that are closely related phylogenetically. For example, if haplotypes are displayed on a phylogenetic (evolutionary) tree a clade includes all haplotypes contained within the same branch.

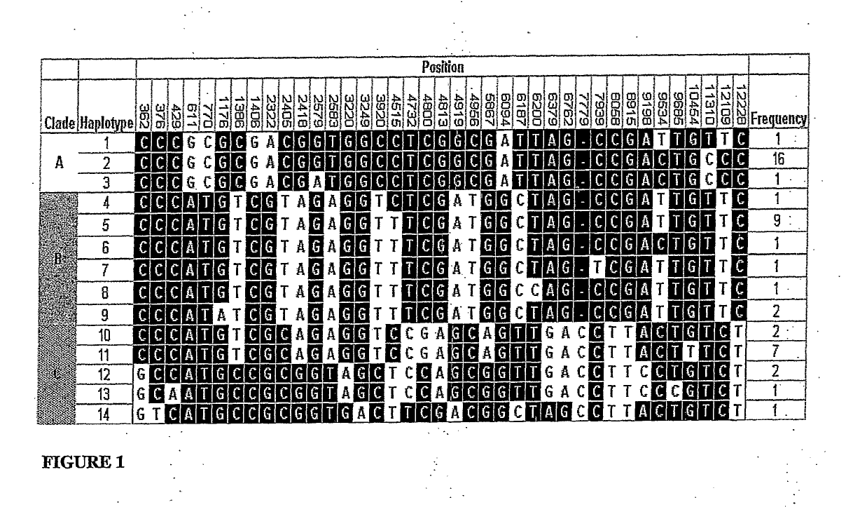

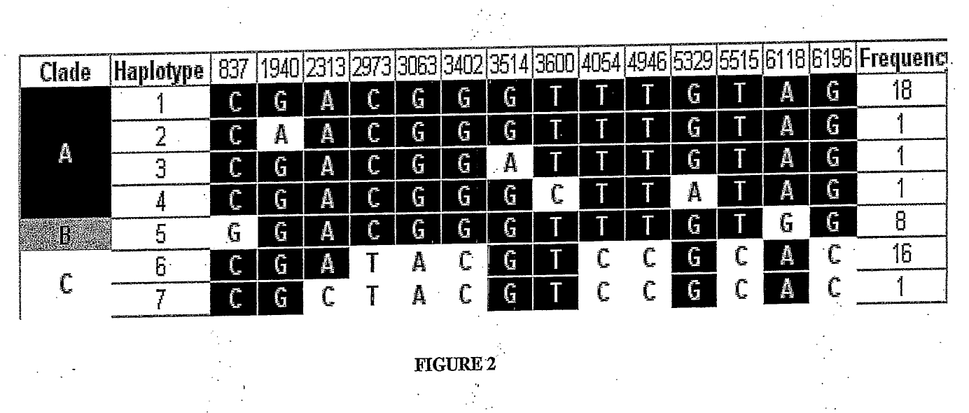

As used herein “haplotype” is a set of alleles of closely linked loci on a chromosome that tend to be inherited together. Such allele sets occur in patterns which are called haplotypes. Haplotype is commonly used in reference to the linked genes of the major histocompatibility complex. “Haplotypes” are also represented as rows in the Table represented in FIGS. 1 and 2. Accordingly, a specific SNP allele at one SNP site is often associated with a specific SNP allele at a nearby second SNP site. When this occurs, the two SNPs are said to be in linkage disequilibrium because the two SNPs are not just randomly associated (in linkage equilibrium).

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping.

Furthermore, the preferential occurrence of a disease gene in association with specific alleles of linked markers, such as SNPs, is called “Linkage Disequilibrium”(LD). This sort of disequilibrium generally implies that most of the disease chromosomes carry the same mutation and the markers being tested are relatively close to the disease gene(s).

In SNP-based association analysis and linkage disequilibrium mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs are tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome.

Accordingly, SNPs as compared with other types of polymorphisms are more likely to be found in close proximity to a genetic locus of interest. SNPs are also mutationally more stable than variable number tandem repeats (VNTRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles-of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as 4732 of SEQ ID NO:1 (see Akey, J. et al. (2001). Haplotypes vs single marker linkage disequilibrium tests: what do we gain? European Journal of lo Human Genetics. 9:291-300; and Zhang, K. et al. (2002). Haplotype block structure and its applications to association studies: power and study designs. American Journal of Human Genetics. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third-locus etc.), the determination of the allele at only one locus would necessarily provide the identity of the allele at the other locus. When evaluating loci for LD those sites within a given population having a high degree of linkage disequilibrium (i.e. an absolute value for D′ of ≧0.5 or r²≧0.5) are potentially useful in predicting the identity of an allele of interest (i.e. associated with the condition of interest). A high degree of linkage disequilibrium may be represented by an absolute value for D′ of ≧0.6 or r²≧0.6. Alternatively, a high degree of linkage disequilibrium may be represented by an absolute value for D′ of ≧0.7 or r²≧0.7 or by an absolute value for D′ of ≧0.8 or r²≧0.8. Additionally, a high degree of linkage disequilibrium may be represented by an absolute value for D′ of ≧0.85 or r²≧0.85 or by an absolute value for D′ of ≧0.9 or r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the haplotype map was created the SNP at position 4054 of SEQ. ID NO.: 2 was in “linkage disequilibrium” with position 6196 of SEQ. ID NO.: 2, whereby when the genotype of 4054 is T the genotype of 6196 is G. Similarly, when the genotype of 4054 is C the genotype of 6196 is C. Accordingly, the determination of the genotype at the 4054 locus of SEQ. ID NO.: 2 will provide the identity of the genotype at 6196 or any other locus in “linkage disequilibrium” therewith. Particularly, where such a locus is has a high degree of linkage disequilibrium thereto.

Linkage disequilibrium is useful for genotype-phenotype association studies. If a specific allele at one SNP site (e.g. “A”) is the cause of a specific clinical outcome (e.g. call this clinical outcome “B”) in a genetic association study then, by mathematical inference, any SNP (e.g. “C”) which is in significant linkage disequilibrium with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on linkage disequilibrium between A and C.

Until the mechanism underlying the genetic contribution to a specific clinical outcome is fully understood, linkage disequilibrium helps identify potential candidate causal SNPs and also helps identify a range of SNPs that may be clinically useful for prognosis of clinical outcome or of treatment effect. If one SNP within a gene is found to be associated with a specific clinical outcome, then other SNPs in linkage disequilibrium will also have some degree of association and therefore some degree of prognostic usefulness. For example, we tested multiple SNPs, having a range of linkage disequilibrium with Protein C SNP 4732, for individual association with 28 day survival in our SIRS/sepsis cohort of ICU patients. We ordered the SNPs by the degree of linkage disequilibrium with Protein C 4732. We found, as expected from the above discussion, that SNPs with high degrees of linkage disequilibrium With Protein C 4732 also had high degrees of association with this specific clinical outcome. As linkage disequilibrium decreased, the degree of association of the SNP with 28 day survival also decreased. These data support the logical conclusion that if A˜B and C˜A, then C˜B. That is, any SNP, whether already discovered or as yet undiscovered, that is in linkage disequilibrium with Protein C 4732 will be a predictor of the same clinical outcomes that Protein C 4732 is a predictor of. The similarity in prediction between this known or unknown SNP and Protein C 4732 will depend on the degree of linkage disequilibrium between this SNP and Protein C 4732.

The “promoter” region is 5′ or upstream of the translation start site, in this case the translation start site is located at position 4062 of SEQ. ID NO: 1 and the transcription start site is, located at position 2559 of SEQ. ID NO: 1.

Numerous sites have been identified as polymorphic sites in the EPCR gene, where those polymorphisms are linked to the polymorphism at position 4054 of SEQ. ID NO: 2 and may also therefore be indicative of subject prognosis. The following single polymorphic sites are linked to 4054 of SEQ. ID NO.: 2:
6196;
5515;
4946;
3402;
3063; and
2973.

It will be appreciated by a person of skill in the art that further linked single polymorphic sites and combined polymorphic sites may be determined. The haplotype of protein C or EPCR can be created by assessing the SNPs of protein C and/or EPCR in normal subjects using a program that has an expectation maximization algorithm (i.e. PHASE). A constructed haplotype of protein C and/or EPCR may be used to find combinations of SNP's that are in linkage disequilibrium (LD) with 4732 of SEQ ID NO: 1 and/or 4054 of SEQ ID NO: 2. Accordingly, the haplotype of an individual could be determined by genotyping other SNPs that are in LD with 4732 of SEQ ID NO: 1 and/or 4054 of SEQ ID NO: 2. Single polymorphic sites or combined polymorphic sites in LD may also be genotyped for assessing subject prognosis.

Previously identified single nucleotide polymorphisms in the protein C gene were described in international patent application, PCT/CA03/00751. Polymorphic sites in the protein C gene previously identified correspond to position 2418 of SEQ ID NO.: 1 or polymorphic sites in linkage disequilibrium thereto. Such polymorphic may also be used as risk genotypes alone or in combination with other Protein C or EPCR gene risk genotypes in determining a subject's suitability for administration the anti-inflammatory agent or the anti-coagulant agent.

The following genotypes for single polymorphic sites in SEQ ID NO: 2 may indicative of a decreased likelihood of recovery from an inflammatory condition or indicative of severe cardiovascular or respiratory dysfunction in critically ill subjects (risk alleles or risk genotypes):
6196 G;
5515 T;
4946 T;
4054 T;
3402 G;
3063 G; and
2973 C.

Whereas the following genotypes for single polymorphic sites in SEQ ID NO: 2 may indicative of a increased likelihood of recovery from an inflammatory condition or indicative of less severe cardiovascular or respiratory dysfunction in critically ill subjects (protective alleles or protective genotypes):
6196 C;
5515 C;
4946 C;
4054 C;
3402 C;
3063 A; and
2973 T.

It will be appreciated by a person of skill in the art, that the numerical designations of the positions of polymorphisms within a sequence are relative to the specific sequence. Also the same positions may be assigned different numerical designations depending on the way in which the sequence is numbered and the sequence chosen, as illustrated by the alternative numbering of equivalent polymorphisms in Foster et al. and Millar et al. above. Furthermore, sequence variations within the population, such as insertions or deletions, may change the relative position and subsequently the numerical designations of particular nucleotides at and around a polymorphic site.

Polymorphic sites in SEQ ID NO:1-47 are identified by their variant designation (i.e. M, W, Y, S, R, K or V).

A representative of a Homo sapiens protein C gene sequence and comprises a sequence as listed in GenBank under accession number AF378903 (SEQ ID NO:1). The SNPs described as −1654 C/T, −1641 A/G and −1476 A/T using the numbering system of Foster et al. correspond to 2405, 2418 and 2583 respectively in SEQ ID. NO:1.

The major and minor alleles for some of the polymorphic sites of the protein C gene are as follows (combination sites are not given here):
at position 4732 the most common nucleotide (major allele) is t and the minor allele is c;
at position 4813 the most common nucleotide (major allele) is g and the minor allele is a;
at position 6379 the most common nucleotide (major allele) is a and the minor allele is g;
at position 9198 the most common nucleotide (major allele) is a and the minor allele is c;
at position 5867 the most common nucleotide (major allele) is g and the minor allele is a;
at position 4800 the most common nucleotide (major allele) is c and the minor allele is g;
at position 3220 the most common nucleotide (major allele) is g and the minor allele is a.

TABLE 1A below shows the flanking sequences for a selection of protein C SNPs giving their relative positions within SEQ ID NO:1, alleles and corresponding SEQ ID NO designations. Each SNP is at position 201 centered within the flanking sequence and identified in bold and underlined.

TABLE 1A

| | | |
|---|---|---|
| Protein C | SEQ ID | |
| SNP | NO: | FLANKING SEQUENCE |

| | | |
|---|---|---|
| G3220A | 3 | TTCCTTGATCTCTGGCCACCAGGGCTATCTCTGTGGCCTTTTGGAGCACC |
| | | TGGTGGTTTGGGGCAGGGGTTGAATTTCCAGGCCTAAAACCACACAGGCC |
| | | TGGCCTTGAGTCCTGGCTCTGCGAGTAATGCATGGATGTAAACATGGAGA |
| | | CCCAGGACCTTGCCTCAGTCTTCCGAGTCTGGTGCCTGCAGTGTACTGAT |
| | | RGTGTGAGACCCTACTCCTGGAGGATGGGGGACAGAATCTGATCGATCCC |
| | | CTGGGTTGGTGACTTCCCTGTGCAATCAACGGAGACCAGCAAGGGTTGGA |
| | | TTTTTAATAAACCACTTAACTCCTCCGAGTCTCAGTTTCCCCCTCTATGA |
| | | AATGGGGTTGACAGCATTAATAACTACCTCTTGGGTGGTTGTGAGCCTTA |
| T4732C | 4 | CCCCTTTCCTGGTCTCCACAGCCAACGGGAGGAGGCCATGATTCTTGGGG |
| | | AGGTCCGCAGGACACATGGGCCCCTAAAGCCACACCAGGCTGTTGGTTTC |
| | | ATTTGTGCCTTTATAGAGCTGTTTATCTGCTTGGGACCTGCACCTCCACC |
| | | CTTTCCCAAGGTGCCCTCAGCTCAGGCATACCCTCCTCTAGGATGCCTTT |
| | | YCCCCCATCCCTTCTTGCTCACACCCCCAACTTGATCTCTCCCTCCTAAC |
| | | TGTGCCCTGCACCCAAGACAGACACTTCACAGAGCCCAGGAGACACCTGG |
| | | GGACCCTTCCTGGGTGATAGGTCTGTCTATCCTCCAGGTGTCCCTGCCCA |
| | | AGGGGAGAAGCATGGGGAATACTTGGTTGGGGGAGGAGAGGAAGACTGGG |
| C4800G | 5 | GGCCCCTAAAGCCACACCAGGCTGTTGGTTTCATTTGTGCCTTTATAGAG |
| | | CTGTTTATCTGCTTGGGACCTGCACCTCCACCCTTTCCCAAGGTGCCCTC |
| | | AGCTCAGGCATACCCTCCTCTAGGATGCCTTTTCCCCCATCCCTTCTTGC |
| | | TCACACCCCCAACTTGATCTCTCCCTCCTAACTGTGCCCTGCACCCAAGA |
| | | SAGACACTTCACAGAGCCCAGGAGACACCTGGGGACCCTTCCTGGGTGAT |
| | | AGGTCTGTCTATCCTCCAGGTGTCCCTGCCCAAGGGGAGAAGCATGGGGA |
| | | ATACTTGGTTGGGGGAGGAGAGGAAGACTGGGGGGATGTGTCAAGATGGG |
| | | GCTGCACGTGGTGTACTGGCAGAAGAGTGAGAGGATTTAACTTGGCAGCC |
| G4813A | 6 | ACACCAGGCTGTTGGTTTCATTTGTGCCTTTATAGAGCTGTTTATCTGCT |
| | | TGGGACCTGCACCTCCACCCTTTCCCAAGGTGCCCTCAGCTGAGGCATAC |
| | | CCTCCTCTAGGATGCCTTTTCCCCCATCCCTTCTTGCTCACACCCCCAAC |
| | | TTGATCTCTCCCTCCTAACTGTGCCCTGCACCCAAGACAGACACTTCACA |
| | | RAGCCCAGGAGACACCTGGGGACCCTTCCTGGGTGATAGGTCTGTCTATC |
| | | CTCCAGGTGTCCCTGCCCAAGGGGAGAAGCATGGGGAATACTTGGTTGGG |
| | | GGAGGARAGGAAGACTGGGGGGATGTGTCAAGATGGGGCTGCAYGTGGTG |
| | | TACTGGCAGAAGAGTGAGAGGATTTAACTTGGCAGCCTTTACAGCAGCAG |
| G5867A | 7 | GGAGTTGTGGGGGTGGCTGAGTGGAGCGATTAGGATGCTGGCCCTATGAT |
| | | GTCGGCCAGGCACATGTGACTGCAAGAAACAGAATTCAGGAAGAAGCTCC |
| | | AGGAAAGAGTGTGGGGTGACCCTAGGTGGGGACTCCCACCAGCCACAGTG |
| | | TAGGTGGTTCAGTCCACCCTCCAGCCACTGCTGAGCACCACTGCCTCCCC |
| | | RTCCCACCTCACAAAGAGGGGACCTAAAGACCACCCTGCTTCCACCCATG |
| | | CCTCTGCTGATCAGGGTGTGTGTGTGACCGAAACTCACTTCTGTCCACAT |
| | | AAAATCGCTCACTCTGTGCCTCACATCAAAGGGAGAAAATCTGATTGTTC |
| | | AGGGGGTCGGAAGACAGGGTCTGTGTCCTATTTGTCTAAGGGTCAGAGTC |
| A6379G | 8 | TCAGCCACYAGGACCTGAAAATTGTGCACGGCCTGGGCCCCCTTCCAAGG |
| | | CATCCAGGGATGCTTTCCAGTGGAGGCTTTCAGGGCAGGAGACCCTCTGG |
| | | CCTGCACCCTCTCTTGCCCTCAGCCTCCACCTCCTTGACTGGACCCCCAT |
| | | CTGGACCTCCATCCCCACCACCTCTTTCCCCAGTGGCCTCCCTGGCAGAC |
| | | RCCACAGTGACTTTCTGCAGGCACATATCTGATCACATCAAGTCCCCACC |
| | | GTGCTCCCACCTCACCCATGGTCTCTCAGCCCCAGCAGGCCTTGGCTGGC |
| | | CTCTCTGATGGAGCAGGCATCAGGCACAGGCCGTGGGTCTCAACGTGGGC |
| | | TGGGTGGTCCTGGACCAGCAGCAGCCGCCGCAGCAGCAACCCTGGTACCT |
| G6762A | 9 | CAGCAACCCTGGTACCTGGTTAGGAACGCAGACCCTCTGCCCCCATCCTC |
| | | CCAACTCTGAAAAACACTGGCTTAGGGAAAGGCGCGATGCTCAGGGGTCC |
| | | CCCAAAGCCCGCAGGCAGAGGGAGTGATGGGACTGGAAGGAGGCCGAGTG |
| | | ACTTGGTGAGGGATTCGGGTCCCTTGCATGCCAGAGGCTGCTGTGGGAGC |
| | | RGACAGTCGCGAGAGCAGCACTGCAGCTGCATGGGGAGAGGGTGTTGCTC |
| | | CAGGGACGTGGGATGGAGGCTGGGCGCGGGCGGGTGGCGCTGGAGGGCGG |
| | | GGGAGGGGCAGGGAGCACCAGCTCCTAGCAGCCAACGACCATCGGGCGTC |
| | | GATCCCTGTTTGTCTGGAAGCCCTCCCCTCCCCTGCCCGCTCACCCGCTG |
| (−)7779C | 10 | GGCCCCTGACGGGGCGCGGCGCGGGGGGCTCAGGAGGGTTTCTAGGGAGG |
| | | GAGCGAGGAACAGAGTTGAGCCTTGGGGCAGCGGCAGACGCGCCCCAACA |
| | | CCGGGGCCACTGTTAGCGCAATCAGCCCGGGAGCTGGGCGCGCCCTCCGC |
| | | TTTCCCTGCTTCCTTTCTTCCTGGCGTCCCCGCCTTCCTCCGGGCGCCCC |
| | | [−/C] |
| | | TGCGCACCTGGGGCCACCTCCTGGAGCGCAAGCCCAGTGGTGGCTCCGCT |
| | | CCCCAGTCTGAGCGTATCTGGGGCGAGGCGTGCAGCGTCCTCCTCCATGT |
| | | AGCCTGGCTGCGTTTTTCTCTGACGTTGTCCGGCGTGCATCGCATTTCCC |
| | | TCTTTACCCCCTTGCTTCCTTGAGGAGAGAACAGAATCCCGATTCTGCC |
| C8058T | 11 | CGTGCAGCGTCCTCCTCCATGTAGCCTGGCTGCGTTTTTCTCTGACGTTG |
| | | TCCGGCGTGCATCGCATTTCCCTCTTTACCCCCTTGCTTCCTTGAGGAGA |
| | | GAACAGAATCCCGATTCTGCCTTCTTCTATATTTTCCTTTTTATGCATTT |
| | | TAATCAAATTTATATATGTATGAAACTTTAAAAATCAGAGTTTTACAACT |
| | | YTTACATTTCAGCATGCTGTTCCTTGGCATGGGTCCTTTTTTCATTCATT |
| | | TTCATTAAAAGGTGGACCCTTTTAATGTGGAAATTCCTATCTTCTGCCTC |
| | | TAGGGACATTTATCACTTATTTCTTCTACAATCTCCCCTTTACTTCCTCT |
| | | ATTTTCTCTTTCTGGACCTCCCATTATTCAGACCTCTTTCCTCTAGTTTT |
| G8915T | 12 | GAGGCTGAGGTGGGAGGATTGCTTGAGCTTGGGAGTTTGAGACTAGCCTG |
| | | GGCAACACAGTGAGACCCTGTCTCTATTTTTAAAAAAAGTAAAAAAAGAT |
| | | CTAAAAATTTAACTTTTTATTTTGAAATAATTAGATATTTCCAGGAAGCT |
| | | GCAAAGAAATGCCTGGTGGGCCTGTTGGCCTGTGGGTTTCCTGCAAGGCC |
| | | KTGGGAAGGCCCTGTCATTGGCAGAACCCCAGATCGTGAGGGCTTTCCTT |
| | | TTAGGCTGCTTTCTAAGAGGACTCCTCCAAGCTCTTGGAGGATGGAAGAC |
| | | GCTCACCCATGGTGTTCGGCCCCTCAGAGCAGGGTGGGGCAGGGGAGCTG |
| | | GTGCCTGTGCAGGCTGTGGACATTTGCATGACTCCCTGTGGTCAGCTAAG |
| A9198C | 13 | CTTGGAGGATGGAAGACGCTCACCCATGGTGTTCGGCCCCTCAGAGCAGG |
| | | GTGGGGCAGGGGAGCTGGTGCCTGTGCAGGCTGTGGACATTTGCATGACT |
| | | CCCTGTGGTCAGCTAAGAGCACCACTCCTTCCTGAAGCGGGGCCTGAAGT |
| | | CCCTAGTCAGAGCCTCTGGTTCACCTTCTGCAGGCAGGGAGAGGGGAGTC |
| | | MAGTCAGTGAGGAGGGCTTTCGCAGTTTCTCTTACAAACTCTCAACATGC |
| | | CCTCCCACCTGCACTGCCTTCCTGGAAGCCCCACAGCCTCCTATGGTTCC |
| | | GTGGTCCAGTCCTTCAGCTTCTGGGCGCCCCCATCACGGGCTGAGATTTT |
| | | TGCTTTCCAGTCTGCCAAGTCAGTTACTGTGTCCATCCATCTGCTGTCAG |
| C12228T | 14 | CTCCCTGGCAGTGCCGTGTTCTGGGGGTCCTCCTCTCTGGGTCTCACTGC |
| | | CCCTGGGGTCTCTCCAGCTACCTTTGCTCCAYGTTCCTTTGTGGCTCTGG |
| | | TCTGTGTCTGGGGTTTCCAGGGGTCTCGGGCTTCCCTGCTGCCCATTCCT |
| | | TCTCTGGTCTCACGGCTCCGTGACTCCTGAAAACCAACCAGCATCCTACC |
| | | YCTTTGGGATTGACACCTGTTGGCCACTCCTTCTGGCAGGAAAAGTCACC |
| | | GTTGATAGGGTTCCACGGCATAGACAGGTGGCTCCGCGCCAGTGCCTGGG |
| | | ACGTGTGGGTGCACAGTCTCCGGGTGAACCTTCTTCAGGCCCTCTGCCCA |
| | | GGCCTGCAGGGGCACAGCAGTGGGTGGGCCTCAGGAAAGTGCCACTGGGG |

The Sequences given in TABLE 1A (SEQ ID NO:3-14) above and in SEQ ID NO:1 would be useful to a person of skill in the art in the design of primers and probes or other oligonucleotides for the identification of protein C SNP alleles and or genotypes as described herein.

A representative Human endothelial cell protein C receptor (EPCR) gene sequence is found at SEQ ID NO:2. The major and minor alleles for some of the primary polymorphic sites of the EPCR gene are as follows:
at position 6196 the most common nucleotide (major allele) is g and the minor allele is c;
at position 5515 the most common nucleotide (major allele) is t and the minor allele is c;
at position 4946 the most common nucleotide (major allele) is t and the minor allele is c;
at position 4054 the most common nucleotide (major allele) is t and the minor allele is c;
at position 3402 the most common nucleotide (major allele) is g and the minor alleles are c and a;
at position 3063 the most common nucleotide (major allele) is g and the minor alleles is a;
at position 2973 the most common nucleotide (major allele) is c and the minor allele is t.

TABLE 1B below shows the flanking sequences for a selection of EPCR SNPs giving their relative positions within SEQ ID NO:2, alleles and corresponding SEQ ID NO designations. Each SNP is at position 201 centered within the Ranking sequence and identified in bold and underlined.

TABLE 1B

| EPCR | SEQ ID | |
|---|---|---|
| SNP | NO: | FLANKING SEQUENCE |
| C2973T | 15 | TGCTTTTCTTTCTTTTTTCTTTTTTTTTTTTTTTTTTTTTTTTGAGATGG |
| | | AGTCTTGTGCTGTCACCCAGGCTGGAATGCAGTGGCACAATTTCAGCTAA |
| | | CTGTAACCTCCAACTCCCAGGTTCAGGCGATTCTCCTGCCTCAGCCTCCT |
| | | GAGTAGCTGGGACTACAGGCATGTACCACCACGCCTGGCTAATATTTGTA |
| | | YTTTTAGTACAGATGGGGTTTCGCCATGTTGGCCAGGCTGGTCTTGAATC |
| | | CCTGACCTCAAGTGATCCGCCCGCCTCGGCCTCCCAAAGTGCTGGGATTA |
| | | CAGGCATGAGCCACCGCGCCCAGTCTCTGAGCTGGGTCTTAAATCATGAA |
| | | TAAACTTCGCCAGGCAGAAAAAGGGAGGCAGAGCAATCCTGACATGCTAT |
| G3063A | 16 | TTTCAGCTAACTGTAACCTCCAACTCCCAGGTTCAGGCGATTCTCCTGCC |
| | | TCAGCCTCCTGAGTAGCTGGGACTACAGGCATGTACCACCACGCCTGGCT |
| | | AATATTTGTACTTTTAGTACAGATGGGGTTTCGCCATGTTGGCCAGGCTG |
| | | GTCTTGAATCCCTGACCTCAAGTGATCCGCCCGCCTCGGCCTCCCAAAGT |
| | | RCTGGGATTACAGGCATGAGCCACCGCGCCCAGTCTCTGAGCTGGGTCTT |
| | | AAATCATGAATAAACTTCGCCAGGCAGAAAAAGGGAGGCAGAGCAATCCT |
| | | GACATGCTATTCATGTGTCAGCCAAAGGCAGCATGAGGAATCCCAACTAG |
| | | TTTGATATATAAGCAGCGGGAAGCGGCCAGAAAAGGCAGCAGGGGCCAGG |
| G3402C | 17 | ATCCCAACTAGTTTGATATATAAGCAGCGGGAAGCGGCCAGAAAAGGCAG |
| | | CAGGGGCCAGGTCTCTAGCAGCCTTGAATGCCAGGCTAAAGACTCTGGAC |
| | | TTGATCCTGTGGGGAGGCAGTGTAGCAGAATGGCTGAGTGCTGGACTTGA |
| | | CTGCCTACGTGCAAACCTTGGCTCTGCTACACTATCTCTGTCTCAGTTTC |
| | | VCATGTAGACTGGGGTTAATAATAGTAGCTATTGCATTAAGCCACTGGGG |
| | | AAAGGCACAAAGATAATAATGTATGTAAAGCCCATTGCCCAGGTTATAAT |
| | | AAGCACTGAATCGACATTGGCTATGATTATTTTTGATTAATGAAGGGGAG |
| | | GGGGTTATGGCACTGGAAGATTTTAAGTAGGAAAAGGACATGATCTCATC |
| T4054C | 18 | AGTTTCCTCATCTGTAAAACGGAGATAATAATCCCTGTCCTGTCCTCCTG |
| | | GCAGAGTTACTGTCAGCGTCAAACGGGAGAAGCGGTGGGAGGGCACATTA |
| | | TAGTTTATGAAGGGTCGAGAAGGCGGGCGGCCAGCCTCGAGGTAGGGGGT |
| | | TATTATCTTCCGCTGCCCGCCGCCCCCTCCCACGCCGGCCCAGGCTGAAG |
| | | YTGACTCTGCCCGCAGGCCTCCAAAGACTTCATATGCTCCAGATCTCCTA |
| | | CTTCCGCGACCCCTATCACGTGTGGTACCAGGGCAACGCGTCGCTGGGGG |
| | | GACACCTAACGCACGTGCTGGAAGGCCCAGACACCAACACCACGATCATT |
| | | CAGCTGCAGCCCTTGCAGGAGCCCGAGAGCTGGGCGCGCACGCAGAGTGG |
| T4946C | 19 | CTCCACTCATGACCCGAACTCTTCCCCCAAAGACCCCAAGTTCTTCTCTC |
| | | AAAGCCCCACTCCTTCCCCGTCACAACCCTAACTCCTTCTTCTCAAAGAC |
| | | CCCAATTTCTTTTCTCAAAGCACCAAGCACCACTCCGTCCCCCTTCCCCC |
| | | ACCATCATGGCCTTTAACTCCTTTCTCTCCTAGTCCCCCACCCCACCCCC |
| | | YTTTTTTTTTTTTTTTTTTTTTTTTGAGACGGAGTCTTGCTCTGTCGTCC |
| | | AGGCTGGAGTGCAGTGGCGCGATCTCGGCTCACTGCAACTTCCGCCTCCC |
| | | GGGTTCAAGCGATTCTCCTGCCTCAGCCTCCCAAGCAGCTGGGACTACAG |
| | | GCACCCGCCACCACGCCCGGCTAATTTTTTGTATTTTTAGTAGAGACGGG |
| T5515C | 20 | TCATCACAGTCCCTGGCCCCTTCTTTCTTAGCCTCTAACAGGCTAACCCC |
| | | AAACCCCTCCTCACAGCCCCAGGCCCTTCTCCCCATAGTTCCCTGACCTA |
| | | GACTCCCCTCTCCTCACAGCACTGACTCTTGCCTTCTCATGTTCTTTTCC |
| | | CCTTGGTGGGCCTCGCCCACACCTGGCACCCTCTCTGCACAGTCCCCTGA |
| | | YCCTGACTGTCTATCCACAGTTCCTCTGACCATCCGCTGCTTCCTGGGCT |
| | | GTGAGCTGCCTCCCGAGGGCTCTAGAGCCCATGTCTTCTTCGAAGTGGCT |
| | | GTGAATGGGAGCTCCTTTGTGAGTTTCCGGCCGGAGAGAGCCTTGTGGCA |
| | | GGCAGACACCCAGGTCACCTCCGGAGTGGTCACCTTCACCCTGCAGCAGC |
| G6196C | 21 | GGGGTTTGACTCAAATCATGGACTCCTTGGGGGCCTATTCTTCGGGCTAA |
| | | CTCTTTGCATGTTCTGCAGGGAGCCAAACAAGCCGCTCCTACACTTCGCT |
| | | GGTCCTGGGCGTCCTGGTGGGCAGTTTCATCATTGCTGGTGTGGCTGTAG |
| | | GCATCTTCCTGTGCACAGGTGGACGGCGATGTTAATTACTCTCCAGCCCC |
| | | STCAGAAGGGGCTGGATTGATGGAGGCTGGCAAGGGAAAGTTTCAGCTCA |
| | | CTGTGAAGCCAGACTCCCCAACTGAAACACCAGAAGGTTTGGAGTGACAG |
| | | CTCCTTTCTTCTCCCACATCTGCCCACTGAAGATTTGAGGGAGGGGAGAT |
| | | GGAGAGGAGAGGTGGACAAAGTACTTGGTTTGCTAAGAACCTAAGAACGT |

The Sequences given in TABLE 1B (SEQ ID NO:15-21) above and in SEQ ID NO:2 would be useful to a person of skill in the art in the design of primers and probes or other oligonucleotides for the identification of EPCR SNP alleles and or genotypes as described herein.

TABLE 1C

| | | | Chromosome/ | | Location |
|---|---|---|---|---|---|
| | | | Contig | | Relative |
| db SNP # | Gene | Chromosome | Accession | SNP | to Gene |
| rs908787 | PROC | 2 | NT_005079 | [C/G] | 5′ |
| rs777566 | PROC | 2 | NT_005079 | [C/G] | 3′ |
| rs334135 | PROC | 2 | NT_005079 | [T/C] | 3′ |
| rs777569 | PROC | 2 | NT_005079 | [A/T] | 3′ |
| rs334142 | PROC | 2 | NT_005079 | [A/G] | 3′ |
| rs334160 | PROC | 2 | NT_005079 | [T/C] | 3′ |
| rs334159 | PROC | 2 | NT_005079 | [T/C] | 3′ |
| rs334151 | PROC | 2 | NT_005079 | [C/T] | 3′ |
| rs334146 | PROC | 2 | NT_005079 | [C/A] | 3′ |
| rs777556 | PROC | 2 | NT_005079 | [C/T] | 3′ |
| rs334144 | PROC | 2 | NT_005079 | [A/C] | 3′ |
| rs2295887 | EPCR | 20 | NT_028392 | [A/G] | 5′ |
| rs1535466 | EPCR | 20 | NT_028392 | [A/G] | 5′ |
| rs1033797 | EPCR | 20 | NT_028392 | [C/T] | 5′ |
| rs1033798 | EPCR | 20 | NT_028392 | [C/T] | 5′ |
| rs1033799 | EPCR | 20 | NT_028392 | [A/C] | 5′ |
| rs2295888 | EPCR | 20 | NT_028392 | [A/G] | 5′ |
| rs666210 | EPCR | 20 | NT_028392 | [C/T] | 5′ |
| rs1415771 | EPCR | 20 | NT_028392 | [A/G] | 5′ |
| rs945959 | EPCR | 20 | NT_028392 | [C/G] | 5′ |
| rs1051056 | EPCR | 20 | NT_028392 | [A/C] | 3′ |
| rs632688 | EPCR | 20 | NT_011387 | [A/C] | 3′ |
| rs633198 | EPCR | 20 | NT_011387 | [C/T] | 3′ |
| rs663550 | EPCR | 20 | NT_011387 | [A/T] | 3′ |

TABLE 1C shows are “rs” identifier number for each of the SNPs identified herein as lying outside of the protein C and EPCR sequences (SEQ ID NOS:1 and 2 respectively). The data base SNP # (db SNP #—have a “rs” prefix designates a:SNP in the database is found at the NCBI SNP database
(http://www.ncbi.nlm.nih.gov/entrez/query.fcgi?db-Snp).

The “rs” numbers are the NCBI|rsSNP ID form. Also shown are the chromosome and contig accession numbers on which the sequences may be found, the alleles at each SNP and the position of the SNP relative to the protein C or EPCR sequence.

TABLE 1D

| | | | |
|---|---|---|---|
| db SNP # | Gene | SNP | SNP including flanking sequence |

| | | | |
|---|---|---|---|
| rs908787 | PROC | [C/G] | gcagaggacccttc[c/g]gccctctgggcagcc |
| | | | SEQ ID NO.:22 |
| rs777566 | PROC | [C/G] | tcctctggaaacag[c/g]ccctccttcatat |
| | | | SEQ ID NO.:23 |
| rs334135 | PROC | [T/C] | cacagccaaaaaag[t/c]gtgaacacaa |
| | | | SEQ ID NO.:24 |
| rs777569 | PROC | [A/T] | tggggaactcactc[a/t]atttccatgctatctct |
| | | | SEQ ID NO.:25 |
| rs334142 | PROC | [A/G] | tttcagttatatcc[a/g]tatttccttga |
| | | | SEQ ID NO.:26 |
| rs334160 | PROC | [T/C] | tagggtcattattt[t/c]gaaactaaaagcagacctgg |
| | | | SEQ ID NO.:27 |
| rs334159 | PROC | [T/C] | acctctcgtgtata[t/c]actctggtagggcc |
| | | | SEQ ID NO.:28 |
| rs33415l | PROC | [C/T] | gaagcgacccagct[c/t]acctcagcagcttca |
| | | | SEQ ID NO.:29 |
| rs334146 | PROC | [C/A] | gacaaatctcttga[c/a]atcagtatatggctggttt |
| | | | SEQ ID NO.:30 |
| rs777556 | PROC | [C/T] | ttgcagttttatta[c/t]gatgtagttaggtgtagatt |
| | | | SEQ ID NO.:31 |
| rs334144 | PROC | [A/C] | ttttgtgtataata[a/c]gtacatatgaaaaacttaaa |
| | | | SEQ ID NO.:32 |
| rs2295887 | EPCR | [A/G] | tatactctgcagtg[a/g]gggagatgggataatggaca |
| | | | SEQ ID NO.:33 |
| rs1535466 | EPCR | [A/G] | ggaacataagatga[a/g]taaggcatggattctgcatt |
| | | | SEQ ID NO.:34 |
| rs1033797 | EPCR | [C/T] | agatgcagggcagg[c/t]gccccagtgcttcttgggaa |
| | | | SEQ ID NO.:35 |
| rs1033798 | EPCR | [C/T] | cacccagcatgtga[c/t]tccactatctgaagacacag |
| | | | SEQ ID NO.:36 |
| rs1033799 | EPCR | [A/C] | ctgacagagtggtt[a/c]taaggagagaaaccgaatag |
| | | | SEQ ID NO.:37 |
| rs2295888 | EPCR | [A/G] | tctttctcctgggt[a/g]tcctgctagagtctgagcca |
| | | | SEQ ID NO.:38 |
| rs666210 | EPCR | [C/T] | agagatttcctctc[c/t]gggcctaaaggtcaaacaac |
| | | | SEQ ID NO.:39 |
| rs1415771 | EPCR | [A/C] | gtaagaattgcggg[a/g]agcgcggctctagctcagct |
| | | | SEQ ID NO.:40 |
| rs945959 | EPCR | [C/G] | aaagggaaaggacc[c/g]ggttcacgcttcccattccc |
| | | | SEQ ID NO.:41 |
| rs1051056 | EPCR | [A/C] | taaacaagtcatcc[a/c]caatcaaaatacaacattca |
| | | | SEQ ID NO.:42 |
| rs632688 | EPCR | [A/C] | cccacccaaacaaa[a/c]aacaaaacccattattttat |
| | | | SEQ ID NO.:43 |
| rs633198 | EPCR | [C/T] | agattagatttggt[c/t]tgtggaattccagggaacag |
| | | | SEQ ID NO.:44 |
| rs663550 | EPCR | [A/T] | acattaaaaaaaaa[a/t]tatttgtttagggtctgtcc |
| | | | SEQ ID NO.:45 |

TABLE 1D shows are “rs” identifier number for each of the SNPs identified in TABLE 1C along with flanking sequences of each SNP.

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding region's (5′ and 3′ to the coding sequence). Such non-coding sequences may contain regulatory sequences needed for transcription and translation of the sequence or introns etc. or may as yet to have any function attributed to them beyond the occurrence of the SNP of interest. For Example, the sequences identified in TABLES 1C and 1D.

A “genotype” is defined as the genetic constitution of an organism, usually in respect to one gene or a few genes or a region of a gene relevant to a particular context (i.e. the genetic loci responsible for a particular phenotype).

Table 1E. below shows a genotype correlation for Protein C and EPCR SNPs with values representing an ability to recover from an inflammatory condition and in indication of responsiveness to treatment of an inflammatory condition with an anti-inflammatory agent or an anti-coagulant agent.

| Position | | Patient Outcome | Responsiveness To |
|---|---|---|---|
| (SEQ ID NO) | Genotype | Score* | Treatment^{∞} |
| 4732 (1) | CC | 0 | R |
| 4732 (1) | CT | 1 | R |
| 4732 (1) | TT | 2 | NR |
| 4813 (1) | AA | 0 | R |
| 4813 (1) | AG | 1 | R |
| 4813 (1) | GG | 2 | NR |
| 6379 (1) | GG | 0 | R |
| 6379 (1) | GA | 1 | R |
| 6379 (1) | AA | 2 | NR |
| 6762 (1) | AA | 0 | R |
| 6762 (1) | AG | 1 | R |
| 6762 (1) | GG | 2 | NR |
| 7779 (1) | CC | 0 | R |
| 7779 (1/46) | C/T | 1 | R |
| 7779 (46) | TT | 2 | NR |
| 8058 (1) | TT | 0 | R |
| 8058 (1) | TC | 1 | R |
| 8058 (1) | CC | 2 | NR |
| 8915 (1) | TT | 0 | R |
| 8915 (1) | TG | 1 | R |
| 8915 (1) | GG | 2 | NR |
| 12228 (1) | TT | 0 | R |
| 12228 (1)‘ | TC | 1 | R |
| 12228 (1) | CC | 2 | NR |
| 2418 (1) | AA | 0 | R |
| 2418 (1) | AG | 1 | R |
| 2418 (1) | GG | 2 | NR |
| 4054 (2) | TT | 0 | R |
| 4054 (2) | TC | 1 | R |
| 4054 (2) | CC | 2 | NR |
| 2973 (2) | CC | 0 | R |
| 2973 (2) | CT | 1 | R |
| 2973 (2) | TT | 2 | NR |
| 3063 (2) | GG | 0 | R |
| 3063 (2) | GA | 1 | R |
| 3063 (2) | AA | 2 | NR |
| 3402 (2) | GG | 0 | R |
| 3402 (2) | GC | 1 | R |
| 3402 (2) | CC | 2 | NR |
| 4946 (2) | TT | 0 | R |
| 4946 (2) | TC | 1 | R |
| 4946 (2) | CC | 2 | NR |
| 5515 (2) | TT | 0 | R |
| 5515 (2) | TC | 1 | R |
| 5515 (2) | CC | 2 | NR |
| 6196 (2) | GG | 0 | R |
| 6196 (2) | GC | 1 | R |
| 6196 (2) | CC | 2 | NR |

*good = 2; moderate = 1; poor = 0.
^{∞}Responsive (R); Non-Responsive (NR).

A “phenotype” is defined as the observable characters of an organism.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine, A “transversion” is the replacement of a purine by a pyrimidine or vice versa. Single nucleotide polymorphisms can also arise from a deletion (represented by “−” or “del”) of a nucleotide or an insertion (represented by “+” or “ins”) of a nucleotide relative to a reference allele. Furthermore, it would be appreciated by a person of skill in the art, that an insertion or deletion within a given sequence could alter the relative position and therefore the position number of another polymorphism within the sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, and D) white blood cell count >12,000 per mm3 or <4,000 mm3. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Subject outcome or prognosis as used herein refers the ability of a subject to recover from an inflammatory condition. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome or prognosis may be accomplished by various methods. For Example, an “APACHE II” score is defined as cute Physiology And Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated protein C (XIGRIS™—drotrecogin alfa (activated)) in the United States is an APACHE II score of ≧25. In Europe, the criteria or indication for administering activated protein C is an APACHE II score of ≧25 or 2 organ system failures.

“Activated protein C” is also known as Drotrecogin alfa (activated) and is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotecogin alfa (activated) is indicated for the reduction of mortality in adult subjects with severe sepsis (sepsis associated with acute organ dysfunction) who have a high risk of death (e.g., as determined by an APACHE II score of greater >25 or having 2 or more organ system failures).

XIGRIS™ is available in 5 mg and 20 mg single-use vials containing sterile, preservative-free, lyophilized drug. The vials contain 5.3 mg and 20.8 mg of drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively. XIGRIS™ is recommended for intravenous administration at an infusion rate of 24 mcg/kg/hr for a total duration of infusion of 96 hours. Dose adjustment based on clinical or laboratory parameters is not recommended. If the infusion is interrupted, it is recommended that when restarted the infusion rate should be 24 mcg/kg/hr. Dose escalation or bolus doses of drotrecogin alfa are not recommended. XIGRIS™ may be reconstituted with Sterile Water for Injection and further diluted with sterile normal saline injection. These solutions must be handled so as to minimize agitation of the solution (Product information. XIGRIS™, Drotecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714 all of which are incorporated herein by reference.

Treatment of sepsis using activated protein C in combination with a bactericidal and endotoxin neutralizing agent is described in U.S. Pat. No. 6,436,397; methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468, all of which are incorporated herein by reference.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAP) were calculated as previously described. For example, acute lung injury was calculated as follows. Acute lung injury is defined as present when a subject meets all of these four criteria, 1) Need for mechanical ventilation,s2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung in over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR)>1.5 are days that a person is alive and does not have an INR>1.5.

TABLE 2A
Brussels Organ Dysfunction Scoring System

| | ORGANS | | | | |
|---|---|---|---|---|---|
| | Free of | | Clinically Significant | | |
| | Organ Dysfunction | | Organ Dysfunction | | |
| | Normal | Mild | Moderate | Severe | Extreme |
| | DAF ORGAN DYSFUNCTION SCORE | | | | |
| | 1 | | 0 | | |
| Cardiovascular | >90 | ≦90 | ≦90 | ≦90 plus | ≦90 plus |
| Systolic BP | | Responsive to | Unresponsive to | pH ≦7.3 | pH ≦7.2 |
| (mmHg) | | fluid | fluid | | |
| Pulmonary | >400 | 400-301 | 300-201 | 200-101 | ≦100 |
| PₐO₂/F_{I}O₂ | | | Acute lung | ARDS | Severe AR |
| (mmHg) | | | injury | | |
| Renal | <1.5 | 1.5-1.9 | 2.0-3.4 | 3.5-4.9 | ≧5.0 |
| Creatinine | | | | | |
| (mg/dL) | | | | | |
| Hepatic | <1.2 | 1.2-1.9 | 2.0-5.9 | 6.0-11.9 | ≧12 |
| Bilirubin | | | | | |
| (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, March 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

2. GENERAL METHODS

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomrerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lug kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by Chomczynski (U.S. Pat. No. 5,945,515), whereby genetic material can be extracted efficiently in as little as twenty minutes. Evans and Hugh (U.S. Pat. No. 5,989,431) describe methods for isolating DNA using a hollow membrane filter.

Once a subject's genetic sequence information has been obtained from the subject it may then be further analyzed to detect or determine the identity or genotype of one or more polymorphisms in a protein C or EPCR sequences and associated sequences. For example, a nucleotide corresponding to position 4732 of SEQ ID NO: 1 or position 4054 of SEQ ID NO: 2. The sequence of interest may also include other protein C or EPCR polymorphisms as described herein. Detection or determination of a nucleotide identity or the genotype of one or more single nucleotide polymorphism(s) (SNP typing), may be accomplished by any one of a number methods or assays known in the art. Many DNA typing methodologies are useful for allelic discrimination and detection of SNPs. Furthermore, the products of allelic discrimination reactions or assays may be detected by one or more detection methods. The majority of SNP genotyping reactions or assays may be assigned to one of four broad groups (allele specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, allele specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and allelic discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, allele specific extensions, or simple PCR amplification) are useful in allelic discrimination reactions. For example, in mini sequencing a primer anneals to its target DNA immediately upstream of the SNP and is extended with a single nucleotide complementary to the polymorphic site. Where the nucleotide is not complementary no extension occurs.

Oligonucleotide ligation assays require two allele specific probes and one common ligation probe per SNP. The common ligation probe hybridizes adjacent to an allele specific probe and when there is a perfect match of the appropriate allele specific probe the ligase joins both allele specific and the common probes. Where there is not a perfect match the ligase is unable to join the allelic specific and common probes.

Alternatively, an invasive cleavage method requires an oligonucleotide called an invader probe and allele specific probes to anneal to the target DNA with an overlap of one nucleotide. When the allele specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the major allele and the other to the minor allele. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragment. Mismatched probes are displaced without fragmentation. The genotype, of a sample is determined by measuring the signal intensity of the two different dyes.

It will be appreciated that numerous other methods for allelic discrimination and detection are known in the art and some of which are described in further detail below. It will also be appreciated that reactions such as arrayed primer extension mini sequencing, tag microarrays and allelic specific extension could be performed on a microarray. One such array based genotyping platform is the microsphere based tag-it high throughput genotyping array (Bortolin S. et al. Clinical Chemistry (2004) 50(11): 2028-36). This method amplifies genomic DNA by PCR followed by allele specific primer extension with universally tagged genotyping primers. The products are then sorted on a Tag-It array and detected using the Luminex xMAP system.

SNP typing methods may include but are not limited to the following:
Restriction Fragment Length Polymorphism (RFLP) strategy—An RFLP gel-based analysis can be used to distinguish between alleles at polymorphic sites within a gene. Briefly, a short segment of DNA (typically several hundred base pairs) is amplified by PCR. Where possible, a specific restriction endonuclease is chosen that cuts the short DNA segment when one variant allele is present but does not cut the short DNA segment when the other allele variant is present. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the initial DNA sample had the allele, which could be cut by the chosen restriction endonuclease. In contrast, if only one higher molecular weight band is observed (at the molecular weight of the PCR product) then the initial DNA sample had the allele variant that could not be cut by the chosen restriction endonuclease. Finally, if both the higher molecular weight band and the two lower molecular weight bands are visible then the initial DNA sample contained both alleles, and therefore the subject was heterozygous for this single nucleotide polymorphism;
Sequencing—For example the Maxam-Gilbert technique for sequencing (Maxam A M. and Gilbert W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by Sanger et al. (Sanger et al Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxy-nucleotides have been used to sequence encephalomyocarditis virus RNA (Zimmern D. and Kaesberg P. Proc. Natl. Acad. Sci. USA (1978) 75(9):4257-4261). Mills D R. and Kramer F R. (Proc. Natl. Acad. Sci. USA (1979) 76(5):2232-2235) describe the use of Qβ replicase and the nucleotide analog inosine for sequencing RNA in a chain-termination mechanism. Direct chemical methods for sequencing RNA are also known (Peattie D A. Proc. Natl. Acad. Sci. USA (1979) 76(4): 1760-1764). Other methods include those of Donis-Keller et al. (1977, Nucl. Acids Res. 4:2527-2538), Simoncsits A. et al. (Nature (1977) 269(5631):833-836), Axelrod V D. et al. (Nucl. Acids Res. (1978) 5(10):3549-3563), and Kramer F R. and Mills D R. (Proc. Natl. Acad. Sci. USA (1978) 75(11):5334-5338, which are incorporated herein by reference). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;
Hybridization methods for the identification of SNPs are described in the U.S. Pat. Nos. 6,270,961 & 6,025,136;
A template-directed dye-terminator incorporation with fluorescent polarization-detection (TDI-FP) method is described by FREEMAN B D. et al. (J Mol Diagnostics (2002) 4(4):209-215) is described for large scale screening;
Oligonucleotide ligation assay (OLA)—is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2): 123 -9; IANNONE M A. et al. Cytometry (2000) 39(2):131-40);
Ligation-Rolling Circle Amplification (L-RCA) has also been successfully used for genotyping single nucleotide polymorphisms as described in QI X. et al. Nucleic Acids Res (2001) 29(22):E116;
5′ nuclease assay has also been successfully used for genotyping single nucleotide polymorphisms (AYDIN A. et al. Biotechniques (2001) (4):920-2, 924, 926-8.);
Polymerase proofreading methods are used to determine SNPs identities, as described in WO 0181631;
Detection of single base pair DNA mutations by enzyme-amplified electronic transduction is described in PATOLSKY F et al. Nat Biotech. (2001) 19(3):253-257;
Gene chip technologies are also known for single nucleotide polymorphism discrimination whereby numerous polymorphisms may be tested for simultaneously on a single array (EP 1120646 and Gilles P N. et al. Nat. Biotechnology (1999) 17(4):365-70);
Matrix assisted laser desorption ionization time of flight (MALDI-TOF) mass spectroscopy is also useful in the genotyping single nucleotide polymorphisms through the analysis of microsequencing products (Haff L A. and Smirnov I P. Nucleic Acids Res. (1997) 25(18):3749-50; Haff L A. and Smirnov I P. Genome Res. (1997) 7:378-388; Sun X. et al. Nucleic Acids Res. (2000) 28 e68; Braun A. et al. Clin. Chem. (1997) 43:1151-1158; Little D P. et al. Eur. J. Clin. Chem. Clin. Biochem. (1997) 35:545-548, Fei Z. et al. Nucleic Acids Res. (2000) 26:2827-2828; and Blondal T. et al. Nucleic Acids Res. (2003) 31(24):e155); or
Allele specific PCR methods have also been successfully used for genotyping single nucleotide polymorphisms (Hawkins J R. et al. Hum Mutat (2002) 19(5):543-553).

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject outcome or prognosis or ability of a subject recover from an inflammatory condition based on the genotype (the nucleotide at the position) of the polymorphism of interest. In the present invention, polymorphisms in protein C sequence and/or polymorphisms in endothelial cell protein C receptor (EPCR) sequence, are used to obtain a prognosis or to determine subject outcome. Methods for obtaining subject outcome or prognosis or for subject screening may be useful to determine the ability of a subject to recover from an inflammatory condition. Alternatively, a single polymorphic site or combined polymorphic sites may be used as an indication of a subject's ability to recover from an inflammatory condition, if they are linked to a polymorphism determined to be indicative of a subject's ability to recover from an inflammatory condition. The method may further comprise comparing the genotype determined for a polymorphism with known genotypes, which are indicative of a prognosis for recovery from the same inflammatory condition as for the subject or another inflammatory condition. Accordingly, a decision regarding the subject's ability to recover may be from an inflammatory condition may be made based on the genotype determined for the polymorphic site.

Once subject outcome or a prognosis is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care. Treatment options that a physician or surgeon may consider in treating a subject with an inflammatory condition may include, but are not limited to the following:
(a) use of anti-inflammatory therapy;
(b) use of steroids;
(c) use of activated Protein C (drotrocogin alpha or XIGRIS™ from Lilly);
(d) use of modulators of the coagulation cascade (such as various versions of heparin) use of antibody to tissue factor;
(e) use of anti-thrombin or anti-thrombin III;
(f) streptokinase;
(g) use of antiplatelet agents such as clopidegrel; and
(h) Surfactant.

Alternative treatments currently in development and potentially useful in the treatment of an inflammatory condition may include, but are not limited to the following: antibodies to tumor necrosis factor (TNF) or even antibody to endotoxin (i.e. lipopolysaccharide, LPS); tumor necrosis factor receptor (TNF); tissue factor pathway inhibitors (tifacogin™ alpha from Chiron); platelet activating factor hydrolase (PAFase™ from ICOS); antibodies to IL6; antibodies, antagonists or inhibitors to high mobility group box 1 (HMGB-1 or HMG-1 tissue plasminogen activator; bradykinin antagonists; antibody to CD-14; interleukin-10; Recombinant soluble tumor necrosis factor receptor-immunoglobulin G1(Roche); Procysteine; Elastase Inhibitor; and human recombinant interleukin 1 receptor antagonist (IL-1 RA).

Methods of treatment of an inflammatory condition in a subject having one or more of the risk genotypes in protein C and/or EPCR associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more single nucleotide polymorphic sites in protein C sequence and/or EPCR sequence. Also, as previously described the sequence identity of one or more single nucleotide polymorphisms in the protein C sequence and EPCR sequence of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

Clinical Phenotype

The primary outcome variable was survival to hospital discharge. Secondary outcome variables were days alive and free of cardiovascular, respiratory, renal, hepatic, hematologic, and neurologic organ system failure as well as days alive and free of SIRS (Systemic Inflammatory Response Syndrome), occurrence of sepsis, and occurrence of septic shock. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count >11,000/μL) (Anonymous. Critical Care Medicine (1992) 20(6):864-74). Subjects were included in this cohort on the calendar day on which the SIRS criteria were met. A subjects' baseline demographics that were recorded included age, gender, whether medical or surgical diagnosis for admission (according to APACHE III diagnostic codes (KNAUS W A et al. Chest (1991) 100(6):1619-36)), and admission APACHE II score. The following additional data were recorded for each 24 hour period (8 am to 8 am) for 28 days to evaluate organ dysfunction, SIRS, sepsis, and septic shock.

Clinically significant organ dysfunction for each organ system was defined as present during a 24 hour period if there was evidence of at least moderate organ dysfunction using the Brussels criteria (TABLE 2A) (RUSSELL J A et al. Critical Care Medicine (2000) 28(10):3405-11). Because data were not always available during each 24 hour period for each organ dysfunction variable, we used the “carry forward” assumption as defined previously (Anonymous. New England Journal of Medicine (2000) 342(18):1301-8). Briefly, for any 24 hour period in which there was no measurement of a variable, we carried forward the “present” or “absent” criteria from the previous 24 hour period. If any variable was never measured, it was assumed to be normal.

To further evaluate cardiovascular, respiratory, and renal function we also recorded, during each 24 hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

To assess duration of organ dysfunction and to correct organ dysfunction scoring for deaths in the 28-day observation period, calculations were made of days alive and free of organ dysfunction (DAF) as previously reported (BERNARD G R et al. New England Journal of Medicine (1997) 336(13):912-8). Briefly, during each 24-hour period for each variable, DAF was scored as 1 if the subject was alive and free of organ dysfunction (normal or mild organ dysfunction, Table 2A). DAF was scored as 0 if the subject had organ dysfunction (moderate, severe, or extreme) or was not alive during that 24-hour period. Each of the 28 days after ICU admission was scored in each subject in this fashion. Thus, the lowest score possible for each variable was zero and the highest score possible was 28. A low score is indicative of more organ dysfunction as there would be fewer days alive and free of organ dysfunction.

Similarly, days alive and free of SIRS (DAF SIRS) were calculated. Each of the four SIRS criteria were recorded as present or absent during each 24 hour period. Presence of SIRS during each 24 hour period was defined by having at least 2 of the 4 SIRS criteria. Sepsis was defined as present during a 24 hour period by having at least two of four SIRS criteria and having a known or suspected infection during the 24 hour period (Anonymous. Critical Care Medicine (1992) 20(6):864-74). Cultures that were judged to be positive due to contamination or colonization were excluded. Septic shock was defined as presence of sepsis plus presence of hypotension (systolic blood pressure <90 mmHg or need for vasopressor agents) during the same 24 hour period.

Haplo Types and Selection of htSNPs

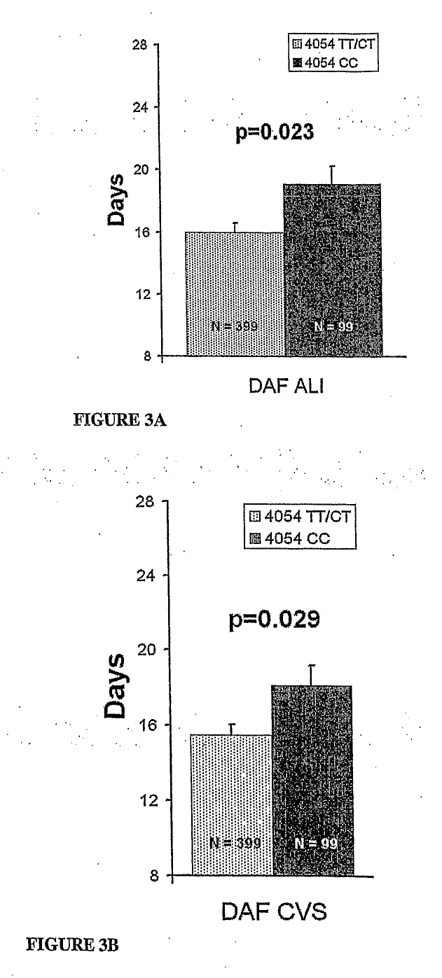

Using unphased Caucasian genotypic data (from pga.mbt.washington.edu (RIEDER M J et al. SeattleSNPs. NHLBI Program for Genomic Applications, UW-FHCRC, Seattle, Wash. (2001)) haplotypes were inferred using PHASE (STEPHENS M. et al. Am J Hum Genet (2001) 68:978-89) software (FIGS. 1 and 2). MEGA 2 (KUMAR S. et al. (2001) 17:1244-5) was then used to infer a phylogenetic tree to identify major haplotype clades for EPCR (FIGS. 3). Haplotypes were sorted according to the phylogenetic tree and haplotype structure was inspected to choose haplotype tag SNPs (htSNPs) (JOHNSON G C. et al. Nat Genet (2001) 29:233-7; and GABRIEL S B. et al. Science (2002) 296:2225-9). htSNPs that identified major haplotype clades of EPCR in Caucasians were chosen. These SNPs were then genotyped in our subject cohort to define haplotypes and haplotype clades.

Blood Collection/Processing Genotyping

The buffy coat was extracted from whole blood and samples transferred into 1.5 ml cryotubes and stored at −80° C. DNA was extracted from the buffy coat of peripheral blood samples using a QIAamp DNA Blood Maxi Kit (Qiagen™). The genotypic analysis was performed in a blinded fashion, without clinical information. Polymorphisms were genotyped using either a Masscode tagging (Qiagen Genomics, Inc—KOKORIS M et al Molecular Diagnosis (2000) 5(4):329-40; BRAY M S. et al. Hum Mutat (2001) 17:296-304.).

Data Collection

Data was recorded for 28 days or until hospital discharge. Raw clinical and laboratory variables were recorded using the worst or most abnormal variable for each 24 hour period with the exception of Glasgow Coma Score, where the best possible score for each 24 hour period was recorded. Missing data on the date of admission was assigned a normal value and missing data after the day one was substituted by carrying forward the previous day's value. Demographic and microbiologic data were recorded. When data collection for each subject was complete, all subject identifiers were removed from all records and the subject file was assigned a unique random number that was cross referenced with the blood samples. The completed raw data file was converted to calculated descriptive and severity of illness scores using standard definitions (i.e. APACHE II and Days alive and free of organ dysfunction calculated using the Brussels criteria).

Statistical Analysis

We used a cohort study design. Rates of dichotomous outcomes (28-day mortality, sepsis and shock at onset of SIRS) were compared between haplotype clades using a chi-squared test, assuming a dominant model of inheritance. Differences in continuous outcome variables between haplotype clades were tested using ANOVA. 28-day mortality was further compared between haplotype clades while adjusting for other confounders (age, sex, and medical vs. surgical diagnosis) using a Cox regression model, together with Kaplan-Meier analysis. Haplotype clade relative risk was calculated. This analysis was performed in the entire cohort, and subsequently in sub-groups of subjects who had sepsis at onset of SIRS, and subjects who had septic shock at onset of SIRS. Genotype distributions were tested for Hardy-Weinberg equilibrium (GUO S W. and THOMPSON E A. (1992) 48:361-72). We report the mean and 95% confidence intervals. Statistical significance was set at p<0.05. The data was analyzed using SPSS 11.5 for Windows™ and SigmaStat 3.0 software (SPSS Inc, Chicago, Ill., 2003).

### 3. EXAMPLES

### Example 1

### EPCR Haplotype Analysis

Inclusion Criteria

500 consecutive critically ill subjects admitted to St. Paul's Hospital Intensive Care Unit (ICU) met the inclusion criteria of having at least two out of four SIRS criteria and were included into our study.

Seven haplotypes of the Endothelial Protein C Receptor (EPCR) gene were inferred using PHASE software as described above and phylogenetic analysis was used to sort these haplotypes into 3 clades. The htSNPs A6118G and T4054C to uniquely identify each haplotype clade (FIG. 2). Of the 500 Caucasian subjects admitted to our ICU with SIRS 498 were successfully genotyped for the A6118G and T4054C polymorphisms and were included in this study. The genotype frequencies of 4054 are shown in Table 3A. These alleles were in Hardy Weinberg equilibrium in our population and there were no significant differences in patient baseline characteristics. FIG. 2 shows 6 additional SNPs of interest (2973, 3063, 3402, 4946, 5515 and 6196) which are all in LD with 4054. The SNP at position 3402 is tri-allelic, but the A allele is not shown in FIG. 2 (only G and C).

TABLE 3A
Genotype frequencies of EPCR haplotype tag SNP T4054C

| | Genotype Frequencies | | | Allele Frequencies | | |
|---|---|---|---|---|---|---|
| | TT | CT | CC | T | C | p* |
| T4054C | 30% | 50% | 20% | 55% | 45% | 0.99 |

*Chi-Squared test for Hardy-Weinberg equilibrium

TABLE 4
Baseline characteristics of critically ill subjects
who had systemic inflammatory response
syndrome by genotype of endothelial protein C receptor T4054C.

| Baseline Characteristics | 4054 TT | 4054 CT | 4054 CC | p Value |
|---|---|---|---|---|
| N | 151 (30%) | 248 (50%) | 99 (20%) | 0.999* |
| Age | 55 ± 1 | 58 ± 1 | 59 ± 2 | 0.095 |
| Gender (% Male) | 29 | 52 | 19 | 0.509 |
| APACHE II | 23 ± 1 | 23 ± 1 | 22 ± 1 | 0.372 |
| Med/Surg | 29 | 48 | 24 | 0.543 |
| Sepsis on Admission | 83 | 83 | 71 | 0.022 |
| Sepsis Anytime | 85 | 85 | 76 | 0.091 |
| Septic Shock on Admission | 52 | 57 | 47 | 0.224 |
| Septic Shock Anytime | 58 | 60 | 53 | 0.435 |

*Exact test for Hardy-Weinberg Equilibrium of Guo and Thompson.

The frequency of the alleles is shown in Table 4. These alleles were in Hardy Weinberg equilibrium in our population. There were no significant differences in baseline characteristics of subjects who had endothelial protein C receptor 4054 T/C (Table 4). Subjects were of similar age, similar gender distribution, and had similar admitting APACE II scores. Approximately eighty percent of these-subjects developed sepsis and 45% of these subjects developed septic shock at some time during their ICU stay.

Haplotype clade 1, defined by 6118A/6196C, occurred with a frequency of 37%. Haplotype clade 2, defined by 6118A/6196G, occurred in 39% of our cohort, while haplotype clade 3, defined by 6118G/6196G, occurred in 24% of our cohort. The EPCR haplotype clades 2 and 3 were associated with fewer days alive and free of acute lung injury/ARDS injury than haplotype clade 1 in our entire cohort of subjects with SIRS. There was also a trend (p<0.07) to more acute renal dysfunction (expressed as fewer days alive and free of acute renal dysfunction) in haplotype clades 2 and 3. These associations were not seen in sub-groups of subjects with sepsis at onset of SIRS, or those subjects with septic shock at onset of SIRS. There was no difference between haplotype clades 1, 2 or 3 in 28 day mortality. There were no associations of EPCR haplotypes with cardiovascular, neurologic, hepatic or coagulation dysfunction. There was also no association of haplotype or genotype with days alive and free of ventilatory, vasopressor or renal support.

When examined individually, it was found that neither htSNP was associated with a difference in baseline characteristics (age, sex, medical vs. surgical diagnosis, APACHE II score), 28-day mortality, or days alive and free of organ dysfunction, with the exception of acute lung injury. The EPCR 6196 G/G genotype was associated with significantly fewer days alive and free of acute lung injury/ARDS than the 6196G/C and C/C genotypes combined (16 days vs. 20 days, p<0.006), again indicating more acute lung injury/ARDS. The 6196 G allele is contained within both haplotype clades 2 and 3.

FIG. 2 shows that the EPCR 4054 SNP is in LD with EPCR 6196. The EPCR 4054 T allele is contained within both haplotype clades 2 and 3 and the C allele is contained within clade 1. FIG. 3A shows day aline and free of acute lung injury for subjects having EPCR 4054 TT/CT genotype (dotted bars) as compared to subjects who had the EPCR 4054 CC genotype (solid bars). Acute lung injury was scored as days alive and free of acute lung injury and subjects who had the EPCR 4054 TT or CT genotype had significantly fewer days alive and free of acute lung injury (p=0.023)

Similarly in FIG. 3B cardiovascular dysfunction and vasopressors use for patients who had the EPCR 4054 TT/CT genotype (dotted bars) is compared to patients who had the EOPCR 4054 CC genotype (solid bars). Cardiovascular dysfunction was scored as days 20 alive and free of cardiovascular dysfunction. Patients who had the EPCR 4054 TT or CT genotype had significantly fewer days alive and free of cardiovascular dysfunction (p=0.029).

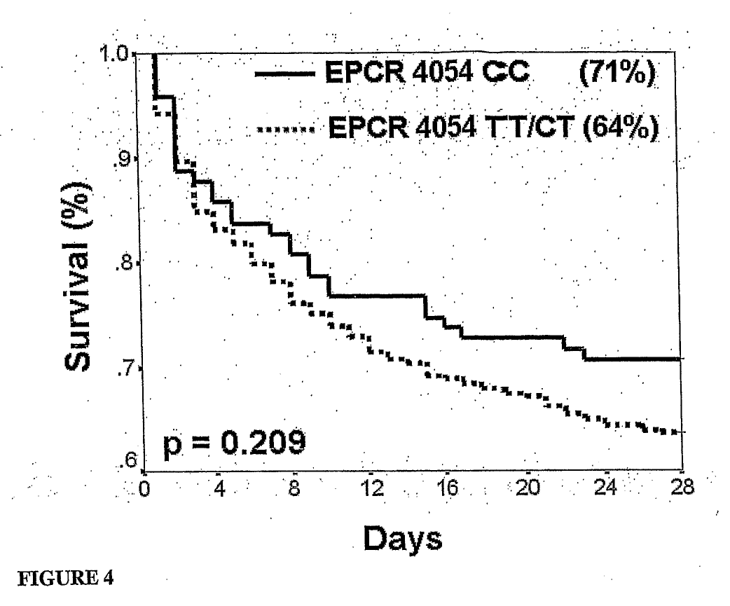

Similar results were found for 28-day survival in subjects who had the T allele of EPCR. 4054 as shown in FIG. 4. Subjects with the EPCR 4054 TT or TC genotype had a survival of 64%, while those with the CC genotype had a 71% survival (p=0.21). Subjects with the EPCR 4054 T allele (TC and TT) had significantly more acute lung injury and strong trend to greater mechanical ventilation as reflected by fewer days alive and free of acute lung injury and mechanical ventilation (Table 5). Subjects who had the T allele of EPCR 4054 (TT and CT) also had more cardiovascular dysfunction as shown by having significantly more days alive and free of cardiovascular dysfunction and a trend to more days alive and free of vasopressor use (Table 5). Subjects who had the T allele of 4054 T/C also had more neurological dysfunction and had a trend to more renal dysfunction (Table 5). Furthermore, the severity of the systemic inflammatory response syndrome was greater m subjects who had the EPCR 4054 T allele as indicated by fewer days alive and free of four of four SIRS criteria (Table 5). Thus, the EPCR 4054 T allele was associated with more acute lung injury and need for mechanical ventilation, worse cardiovascular and neurologic dysfunction, more severe systemic inflammatory response syndrome and trends to worse renal function.

TABLE 5
Days Alive and Free (DAF) of organ dysfunction and severe systemic
inflammatory response syndrome (4 out of 4 criteria)
by 4054 T/C genotype of EPCR in critically ill
subjects who had systemic inflammatory response syndrome.

| Days Alive and | EPCR4054 TT | | |
|---|---|---|---|
| Free | and CT | EPCR4054 CC | p Value |
| Acute Lung Injury | 16 ± 11.4 | 19.1 ± 11.6 | 0.023* |
| Mechanical | 12.5 ± 11.5 | 15 ± 11.7 | 0.058 |
| Ventilation | | | |
| Cardiovascular | 15.5 ± 11.2 | 18.1 ± 10.9 | 0.029 |
| Vasopressor Use | 17.6 ± 11.3 | 19.7 ± 10.9 | 0.086 |
| CNS | 18.3 ± 11.2 | 20.3 ± 10.8 | 0.014 |
| Renal | 16.7 ± 11.8 | 19 ± 11.4 | 0.081 |
| Hepatic | 19.2 ± 11.1 | 20 ± 11.4 | 0.419 |
| SIRS 4 of 4 | 18.5 ± 10.5 | 20.2 ± 10.4 | 0.052 |

*Significance tested by Spearman's Rho correlation.

### Example 2

### Subject Outcome or Prognosis for 4732 Protein C

Polymorphisms

Fourteen haplotypes of the Protein C gene were inferred using PHASE software as described above and phylogenetic analysis was used to sort these haplotypes into 3 clades as shown in FIG. 1. FIG. 1 also shows 8 SNPs in LD which identify lade C (4732, 4813, 6379, 6762, 7779, 8058, 8915 and 12228). Additionally, either of SNPs 3220, 9198 in combination with either, 4800 and 5867 are in LD with 4732 and are unique to clade C.

Table 6 shows the genotype frequencies of T4732C. These alleles were in Hardy Weinberg equilibrium in our population.

TABLE 6
Genotype frequencies of ProC haplotype tag SNP T4732C

| | Genotype Frequencies | | | Allele Frequencies | | |
|---|---|---|---|---|---|---|
| | TT | CT | CC | T | C | p* |
| T4732C | 57% | 37% | 6% | 76% | 24% | 0.99 |

*Chi-Squared test for Hardy-Weinberg equilibrium

It was found that SNP haplotypes of protein C 4732 are associated with altered survival and organ dysfunction in critically ill adults who have systemic inflammatory response syndrome (SIRS).

For Tables 7A and 7B below an inception cohort of 500 Caucasian subjects were studied in ICU who met at least 2/4 criteria for SIRS and defined subgroups of subjects who had sepsis or septic shock. Baseline variables were age, gender, APACHE II and medical vs. surgical reason for ICU admission. The 28-day survival (Kaplan Meier) was determined and severity of organ dysfunction (by Brussels score) was scored by calculating days alive and free (DAP) of organ dysfunction (respiratory, acute lung injury, cardiovascular, vasopressors, renal, coagulation, International Normalized Ratio for Partial Thromboplastin Time (INR), hepatic, and neurological (CNS) as well as systemic inflammatory response syndrome (SIRS with all 4 of 4 criteria (SIRS 4 of 4))) over 28 days. PHASE and MEGA 2 were used to determine the haplotypes of protein C in Caucasians. We then genotyped haplotype tag SNP's that tagged each of the major haplotype clades of each subject.

Subjects were well matched by genotype and haplotype at baseline. It was found that there were 3 major haplotype clades of protein C (37, 39, 24%).

A novel clade was tagged by protein C T 4732 C and was associated with decreased 28-day survival (54%, 60% vs. 68%, 4732 CC, CT, and TT respectively, p<0.05 by Fisher's Exact Test) and with increased severity (measured as fewer DAF) of vasopressor use, renal, coagulation (platelets), INR, and hepatic dysfunction (all preceding have p<0.05) as well as more severe renal dysfunction (Spearman's rho) (See Table 7A below).

TABLE 7A
Days alive and free (DAF) of vasopressors, coagulation (platelets), INR,
renal, hepatic SIRS 4 of 4 and neurological (CNS) dysfunction in critically ill subjects
who had Systemic Inflammatory Response Syndrome (SIRS)

| Genotype of | DAF | DAF | | |
|---|---|---|---|---|
| Protein C 4732 | Vasopressors | Coagulation | DAF INR | DAF Renal |
| PC 4732 TT | 18.8 ± 11.1 | 19.8 ± 11.1 | 19.1 ± 11.3 | 17.8 ± 11.6 |
| PC 4732 CT | 16.9 ± 11.4 | 18.5 ± 11.2 | 18 ± 11.4 | 15.7 ± 12.2 |
| PC 4732 CC | 15.6 ± 11.2 | 16.9 ± 10.6 | 16.8 ± 10.9 | 15.8 ± 10.5 |
| P value | <0.05 | <0.06 | <0.05 | <0.10 |
| | Mean ± Standard | Mean ± Standard | Mean ± Standard | Mean ± Standard |
| | Deviation | Deviation | Deviation | Deviation |

| Genotype of | | | |
|---|---|---|---|
| Protein C 4732 | DAF Hepatic | DAF SIRS 4 of 4 | DAF CNS |
| PC 4732 TT | 20.1 ± 11 | 19.5 ± 10.4 | 19.3 ± 11 |
| PC 4732 CT | 18.8 ± 11.2 | 18 ± 10.7 | 17.9 ± 11.4 |
| PC 4732 CC | 15.5 ± 12 | 16.2 ± 10.3 | 16.1 ± 11.7 |
| p | <0.06 | <0.05 | <0.11 |
| | Mean ± Standard | Mean ± Standard | Mean ± Standard |
| | Deviation | Deviation | Deviation |

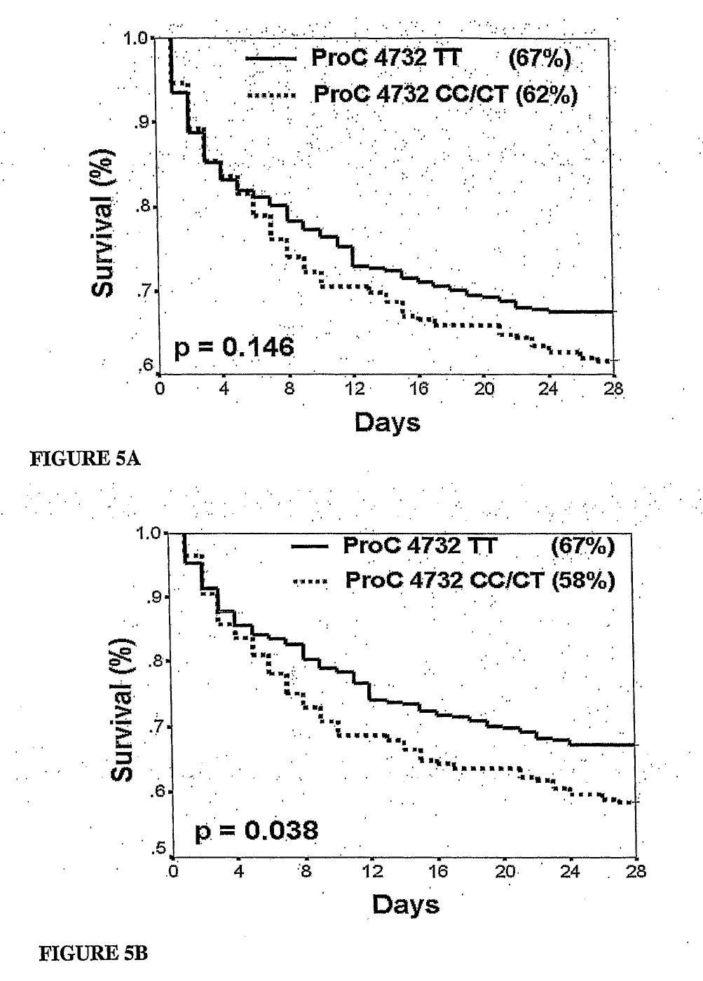

The association of protein C 4732 C with decreased 28 day survival (62% vs. 67% and 58% vs. 67%, for protein C 4732 CC/CT vs. protein C 4732 TT, in SIRS and Sepsis patients in FIGS. 5A/5B respectively) and increased organ dysfunction (use of vasopressors, coagulation (platelets), INR, renal, hepatic SIRS 4 of 4, neurological (CNS) dysfunction and use of inotropic agents (inotropes) was especially pronounced in subjects (n=395 Caucasians) who had sepsis (See Table 7B below).

TABLE 7B
Days alive and free (DAF) of vasopressors, coagulation (platelets), INR,
renal, hepatic dysfunction, SIRS 4 of 4 criteria, neurological (CNS) dysfunction and
use of inotropic agents (Inotropes) in critically ill subjects who had Sepsis

| Genotype of | | | | |
|---|---|---|---|---|
| Protein C | DAF | DAF | | |
| 4732 | Vasopressors | Coagulation | DAF INR | DAF Renal |
| PC 4732 TT | 18.6 ± 10.9 | 20.2 ± 10.7 | 19.3 ± 11.1 | 18 ± 11.3 |
| PC 4732 CT | 16 ± 11.3 | 17.8 ± 11.3 | 17.3 ± 11.4 | 14.9 ± 12 |
| PC 4732 CC | 15.9 ± 10.6 | 17.2 ± 10.1 | 17 ± 10.4 | 15.8 ± 9.9 |
| P value | <0.01 | <0.01 | <0.012 | <0.02 |
| | Mean ± Standard | Mean ± Standard | Mean ± Standard | Mean ± Standard |
| | Deviation | Deviation | Deviation | Deviation |

| | | | | DAF |
|---|---|---|---|---|
| | DAF Hepatic | DAF SIRS 4 of 4 | DAF CNS | Inotropes |
| PC 4732 TT | 20.4 ± 10.7 | 19.4 ± 10.1 | 19.4 ± 10.5 | 20.7 ± 10.3 |
| PC 4732 CT | 18 ± 11.1 | 17.2 ± 10.6 | 17 ± 11.4 | 18.6 ± 11.3 |
| PC 4732 CC | 15.4 ± 11.9 | 16.4 ± 9.6 | 16.3 ± 11.3 | 19.5 ± 10.3 |
| P value | <0.008 | <0.01 | <0.06 | <0.05 |
| | Mean ± Standard | Mean ± Standard | Mean ± Standard | |
| | Deviation | Deviation | Deviation | |

A novel clade of protein C tagged by protein C 4732 C is a useful predictor decreased survival and increased multiple organ dysfunctions in SIRS and in sepsis.

In a subsequent analysis of an increased subject group, 690 Caucasians subjects admitted to our ICU with sepsis and enrolled in this study, 518 were successfully genotyped for the protein C 4732 T/C polymorphism and were included in this analysis. The frequency of the alleles is shown in Table 8A. These alleles were in Hardy-Weinberg equilibrium in our population. There were no significant differences in baseline characteristics of subjects who had the C allele (CC or CT genotype) and those who did not (TT genotype) (Table 8A). Subjects were of similar age, gender distribution, had similar APACHE II scores on admission to the ICU and were admitted with similar ratios of medical/surgical diagnoses.

For the data shown in Tables 8A and 8B below inclusion criteria for subjects was as follows. All subjects admitted to the ICU of St. Paul's Hospital were screened for inclusion. The ICU is a mixed medical-surgical ICU in a tertiary care, university-affiliated teaching hospital. Subjects were included in the study if they met at least two out of four SIRS criteria: 1) fever (>38° C.) or hypothermia (<36° C.), 2) tachycardia (>90 beats/minute), 3) tachypnea (>20 breaths/minute), PaCO₂<32 mm Hg, or need for mechanical ventilation, and 4) leukocytosis (total leukocyte count>12,000 mm³) or leukopenia (<4,000 mm³). Subjects were included in the cohort on the calendar day on which the SIRS criteria were met. Subject who had sepsis were defined by having at least 2 of 4 SIRS criteria plus suspected or documented infection plus one new organ dysfunction believed to be secondary to sepsis (according to definitions of the Society of Critical Care Medicine). Subjects were excluded if blood could not be obtained for genotype analysis. Otherwise the procedures for determining clinical phenotype, haplotype, genotypes and data analysis were as described above.

Table 8A shows the genotype frequencies of T4732C and baseline characteristics of critically ill subjects who had sepsis by genotype of the protein C 4732 T/C polymorphism. These alleles were in Hardy Weinberg equilibrium in our population. There were no significant differences in baseline characteristics of subjects who had the C allele (CC or CT genotype) and those who did not (TT genotype). Subjects were of similar age, gender distribution, had similar APACHE II scores on admission to the ICU and were admitted with similar ratios of medical/surgical diagnoses.

TABLE 8A

| Baseline | | | | |
|---|---|---|---|---|
| Characteristic | 4732 TT | 4732 CT | 4732 CC | p Value |
| N (%) | 289 (56%) | 199 (38%) | 30 (6%) | 0.099* |
| Age (Years) | 58 ± 1 | 57 ± 1 | 58 ± 3 | 0.742 |
| Gender (% Male) | 65 | 67 | 70 | 0.826 |
| APACHE II Score | 23 ± 1 | 25 ± 1 | 24 ± 1 | 0.127 |
| Medical/Surgical | 25 | 25 | 33 | 0.596 |
| Diagnosis (% | | | | |
| Surgical) | | | | |

*Exact test for Hardy-Weinberg equilibrium of Guo and Thompson.

Subjects who had the C allele of the protein C 4732 T/C polymorphism had significantly lower 28-day survival, as indicated by a two-way Fisher exact test (Table 8A) or a Kaplan-Meier 28-day survival curve (FIG. 5B). FIG. 5A shows a Kaplan-Meier 28-day survival curve for protein C 4732 T/C in the inception cohort of 690 critically ill Caucasians with SIRS. FIG. 5B shows a Kaplan-Meier 28-day survival curve for protein C 4732 T/C in the inception cohort of 690 critically ill Caucasians with Sepsis. Sepsis subjects with the C allele of the protein C 4732 TC polymorphism (CC or CT genotype) had a 28-day survival rate of 58% (FIG. 5B), while those without the C allele (TT genotype) had a 28-day survival rate of 67% (p=0.038). Patients having at least one copy of the risk allele (4732 C) have a decreased 28 day survival in both critically ill patients with Sepsis or SIRS.

Table 8B shows Survival and days alive and free (DAF) of organ dysfunction by protein C 4732 T/C genotype in critically ill subjects with sepsis.

TABLE 8B

| | 4732 CC/CT | 4732 TT | p Value |
|---|---|---|---|
| Survival | 58% | 67% | 0.035 |
| DAF | 16 ± 1 | 18 ± 1 | 0.011* |
| Vasopressors | | | |
| DAF Inotropes | 19 ± 1 | 21 ± 1 | 0.040 |
| DAF 3/4 SIRS | 12 ± 1 | 14 ± 1 | 0.034 |
| DAF 4/4 SIRS | 17 ± 1 | 19 ± 1 | 0.011 |
| DAF | 18 ± 1 | 20 ± 1 | 0.009 |
| Coagulation | | | |
| DAF INR >1.5 | 17 ± 1 | 19 ± 1 | 0.014 |
| DAF | 11 ± 1 | 13 ± 1 | 0.088 |
| Ventilation | | | |
| DAF Renal | 15 ± 1 | 17 ± 1 | 0.060 |
| Dysfunction | | | |
| DAF CNS | 17 ± 1 | 19 ± 1 | 0.092 |
| Dysfunction | | | |
| DAF Hepatic | 17 ± 1 | 20 ± 1 | 0.006 |
| Dysfunction | | | |

*Significance tested by Spearman's Rho correlation.

Subjects with the CC or CT genotype of the protein C 4732 polymorphism had significantly greater vasopressor and inotrope use, as indicated by significantly fewer days alive and free of vasopressor and inotrope use. Subjects with the CC or CT genotype of the protein C 4732 polymorphism had significantly greater inflammation as indicated by significantly fewer days alive and free of 3/4 and 4/4 SIRS criteria. Subjects with the CC or CT genotype of the protein C 4732 T/C polymorphism had significantly greater coagulation as indicated by significantly fewer days alive and free of coagulation and days alive and free of INR>1.5. Subjects with the C allele of the protein C 4732 T/C polymorphism had trends toward greater ventilation and greater renal dysfunction as indicated by fewer days alive and free of mechanical ventilation and renal dysfunction. Subjects who had the CC or CT genotype of the protein C 4732 T/C polymorphism had a trend toward greater neurological dysfunction and had significantly greater hepatic dysfunction as indicated by fewer days alive and free of CNS and hepatic dysfunction.

Linkage Disequilibrium of 4732 and 4800

Protein C SNP 4800, which is in LD with Protein C SNP 4732 (r² value of 0.85) was also evaluated within the same patient population as 4732 and also found to provide significant predictions of patient outcome as set out in TABLES 8C, 8D and 8E below.

Table 8C shows p-value comparisons of SIRs patients for Protein C C4732T (reference SNP) and C4800G, which is in linage disequilibrium (LD) with the reference SNP (r² value of 0.85). Also shown are the individual variables for which the p values were generated and the statistical method of analysis for each.

| | | C4732T | C4800G |
|---|---|---|---|
| VARIABLE | STATISTICAL METHOD | p Value | p Value |
| 28-Day Survival by Allele | Fisher's 2-Way Exact Test | 0.036 | 0.146 |
| 28-Day Survival by Allele | KM w Log-Rank Test | 0.039 | 0.146 |
| 28-Day Survival by Allele | Cox Regression | 0.272 | 0.433 |
| 28-Day Survival (ACN) | Linear-by-Linear Association | 0.020 | 0.058 |
| 28-Day Survival (ACN) | KM Log-Rank Test w Trend | 0.025 | 0.070 |
| 28-Day Survival (ACN) | Cox Regression | 0.199 | 0.144 |
| DAF ALI | Spearman's Rho Correlation | 0.060 | 0.399 |
| DAF Pressors | Spearman's Rho Correlation | 0.004 | 0.086 |
| DAF Inotropes | Spearman's Rho Correlation | 0.049 | 0.195 |
| DAF 4/4 SIRS | Spearman's Rho Correlation | 0.008 | 0.060 |
| DAF 3/4 SIRS | Spearman's Rho Correlation | 0.023 | 0.178 |
| DAF Steroid Use | Spearman's Rho Correlation | 0.179 | 0.511 |
| DAF CVS Dysfunction | Spearman's Rho Correlation | 0.063 | 0.389 |
| DAF Resp Dysfunction | Spearman's Rho Correlation | 0.126 | 0.350 |
| DAF P/F <300 | Spearman's Rho Correlation | 0.989 | 0.856 |
| DAF Ventilation | Spearman's Rho Correlation | 0.034 | 0.240 |
| DAF CNS Dysfunction | Spearman's Rho Correlation | 0.049 | 0.126 |
| DAF Coagulation | Spearman's Rho Correlation | 0.081 | 0.119 |
| DAF INR >1.5 | Spearman's Rho Correlation | 0.038 | 0.123 |
| DAF Renal Dysfunction | Spearman's Rho Correlation | 0.009 | 0.066 |
| DAF Renal Support | Spearman's Rho Correlation | 0.075 | 0.171 |
| DAF Hepatic Dysfunction | Spearman's Rho Correlation | 0.090 | 0.129 |
| DAF ALI (ACN) | Spearman's Rho Correlation | 0.022 | 0.384 |
| DAF Pressors (ACN) | Spearman's Rho Correlation | 0.003 | 0.069 |
| DAF Inotropes (ACN) | Spearman's Rho Correlation | 0.034 | 0.162 |
| DAF 4/4 SIRS (ACN) | Spearman's Rho Correlation | 0.004 | 0.041 |
| DAF 3/4 SIRS (ACN) | Spearman's Rho Correlation | 0.010 | 0.147 |
| DAF Steroid Use (ACN) | Spearman's Rho Correlation | 0.074 | 0.585 |
| DAF CVS Dysfunction (ACN) | Spearman's Rho Correlation | 0.018 | 0.348 |
| DAF Resp Dysfunction (ACN) | Spearman's Rho Correlation | 0.073 | 0.275 |
| DAF P/F <300 (ACN) | Spearman's Rho Correlation | 0.906 | 0.780 |
| DAF Ventilation (ACN) | Spearman's Rho Correlation | 0.017 | 0.188 |
| DAF CNS Dysfunction (ACN) | Spearman's Rho Correlation | 0.007 | 0.095 |
| DAF Coagulation (ACN) | Spearman's Rho Correlation | 0.054 | 0.091 |
| DAF INR >1.5 (ACN) | Spearman's Rho Correlation | 0.049 | 0.113 |
| DAF Renal Dysfunction (ACN) | Spearman's Rho Correlation | 0.008 | 0.064 |
| DAF Renal Support (ACN) | Spearman's Rho Correlation | 0.058 | 0.124 |
| DAF Hepatic Dysfunction (ACN) | Spearman's Rho Correlation | 0.060 | 0.103 |

Legend:
by Allele Copy Number (ACN), Kaplan-Meier (KM)

Table 8D shows p-value comparisons of Sepsis patients for Protein C C4732T (reference SNP) and C4800G, which is in linkage disequilibrium (LD) with the reference SNP (r² value of 0.85). Also shown are the individual variables for which the p values were generated and the statistical method of analysis for each.

| | | C4732T | C4800G |
|---|---|---|---|
| VARIABLE | STATISTICAL METHOD | p Value | p Value |
| 28-Day Survival by Allele | Fisher's 2-Way Exact Test | 0.039 | 0.056 |
| 28-Day Survival by Allele | KM w Log-Rank Test | 0.035 | 0.043 |
| 28-Day Survival by Allele | Cox Regression | 0.252 | 0.205 |
| 28-Day Survival (ACN) | Linear-by-Linear Association | 0.035 | 0.024 |
| 28-Day Survival (ACN) | KM Log-Rank Test w Trend | 0.038 | 0.022 |
| 28-Day Survival (ACN) | Cox Regression | 0.204 | 0.075 |
| DAF ALI | Spearman's Rho Correlation | 0.085 | 0.214 |
| DAF Pressors | Spearman's Rho Correlation | 0.004 | 0.021 |
| DAF Inotropes | Spearman's Rho Correlation | 0.018 | 0.037 |
| DAF 4/4 SIRS | Spearman's Rho Correlation | 0.008 | 0.010 |
| DAF 3/4 SIRS | Spearman's Rho Correlation | 0.031 | 0.052 |
| DAF Steroid Use | Spearman's Rho Correlation | 0.247 | 0.420 |
| DAF CVS Dysfunction | Spearman's Rho Correlation | 0.069 | 0.135 |
| DAF Resp Dysfunction | Spearman's Rho Correlation | 0.158 | 0.195 |
| DAF P/F <300 | Spearman's Rho Correlation | 0.891 | 0.523 |
| DAF Ventilation | Spearman's Rho Correlation | 0.099 | 0.188 |
| DAF CNS Dysfunction | Spearman's Rho Correlation | 0.041 | 0.027 |
| DAF Coagulation | Spearman's Rho Correlation | 0.035 | 0.014 |
| DAF INR >1.5 | Spearman's Rho Correlation | 0.017 | 0.032 |
| DAF Renal Dysfunction | Spearman's Rho Correlation | 0.006 | 0.007 |
| DAF Renal Support | Spearman's Rho Correlation | 0.071 | 0.042 |
| DAF Hepatic Dysfunction | Spearman's Rho Correlation | 0.035 | 0.011 |
| DAF ALI (ACN) | Spearman's Rho Correlation | 0.079 | 0.203 |
| DAF Pressors (ACN) | Spearman's Rho Correlation | 0.006 | 0.015 |
| DAF Inotropes (ACN) | Spearman's Rho Correlation | 0.012 | 0.031 |
| DAF 4/4 SIRS (ACN) | Spearman's Rho Correlation | 0.006 | 0.006 |
| DAF 3/4 SIRS (ACN) | Spearman's Rho Correlation | 0.032 | 0.037 |
| DAF Steroid Use (ACN) | Spearman's Rho Correlation | 0.250 | 0.475 |
| DAF CVS Dysfunction (ACN) | Spearman's Rho Correlation | 0.051 | 0.117 |
| DAF Resp Dysfunction (ACN) | Spearman's Rho Correlation | 0.194 | 0.140 |
| DAF P/F <300 (ACN) | Spearman's Rho Correlation | 0.791 | 0.430 |
| DAF Ventilation (ACN) | Spearman's Rho Correlation | 0.126 | 0.133 |
| DAF CNS Dysfunction (ACN) | Spearman's Rho Correlation | 0.020 | 0.020 |
| DAF Coagulation (ACN) | Spearman's Rho Correlation | 0.030 | 0.010 |
| DAF INR >1.5 (ACN) | Spearman's Rho Correlation | 0.035 | 0.028 |
| DAF Renal Dysfunction (ACN) | Spearman's Rho Correlation | 0.005 | 0.007 |
| DAF Renal Support (ACN) | Spearman's Rho Correlation | 0.088 | 0.027 |
| DAF Hepatic Dysfunction (ACN) | Spearman's Rho Correlation | 0.027 | 0.008 |

Legend:
by Allele Copy Number (ACN), Kaplan-Meier (KM)

Table 8E shows p-value comparisons of Septic Shock patients for Protein C C4732T (reference SNP) and C4800G, which is in linkage disequilibrium (LD) with the reference SNP (r² value of 0.85). Also shown are the individual variables for which the p values were generated and the statistical method of analysis for each.

| | | C4732T | C4800G |
|---|---|---|---|
| VARIABLE | STATISTICAL METHOD | p Value | p Value |
| 28-Day Survival by Allele | Fisher's 2-Way Exact Test | 0.106 | 0.059 |
| 28-Day Survival by Allele | KM w Log-Rank Test | 0.087 | 0.041 |
| 28-Day Survival by Allele | Cox Regression | 0.088 | 0.088 |
| 28-Day Survival (ACN) | Linear-by-Linear Association | 0.117 | 0.023 |
| 28-Day Survival (ACN) | KM Log-Rank Test w Trend | 0.122 | 0.023 |
| 28-Day Survival (ACN) | Cox Regression | 0.061 | 0.025 |
| DAF ALI | Spearman's Rho Correlation | 0.534 | 0.252 |
| DAF Pressors | Spearman's Rho Correlation | 0.075 | 0.022 |
| DAF Inotropes | Spearman's Rho Correlation | 0.085 | 0.037 |
| DAF 4/4 SIRS | Spearman's Rho Correlation | 0.017 | 0.002 |
| DAF 3/4 SIRS | Spearman's Rho Correlation | 0.047 | 0.010 |
| DAF Steroid Use | Spearman's Rho Correlation | 0.866 | 0.752 |
| DAF CVS Dysfunction | Spearman's Rho Correlation | 0.362 | 0.081 |
| DAF Resp Dysfunction | Spearman's Rho Correlation | 0.422 | 0.167 |
| DAF P/F <300 | Spearman's Rho Correlation | 0.811 | 0.758 |
| DAF Ventilation | Spearman's Rho Correlation | 0.256 | 0.131 |
| DAF CNS Dysfunction | Spearman's Rho Correlation | 0.072 | 0.010 |
| DAF Coagulation | Spearman's Rho Correlation | 0.079 | 0.026 |
| DAF INR >1.5 | Spearman's Rho Correlation | 0.032 | 0.014 |
| DAF Renal Dysfunction | Spearman's Rho Correlation | 0.046 | 0.008 |
| DAF Renal Support | Spearman's Rho Correlation | 0.525 | 0.095 |
| DAF Hepatic Dysfunction | Spearman's Rho Correlation | 0.055 | 0.009 |
| DAF ALI (ACN) | Spearman's Rho Correlation | 0.674 | 0.272 |
| DAF Pressors (ACN) | Spearman's Rho Correlation | 0.074 | 0.016 |
| DAF Inotropes (ACN) | Spearman's Rho Correlation | 0.120 | 0.031 |
| DAF 4/4 SIRS (ACN) | Spearman's Rho Correlation | 0.015 | 0.001 |
| DAF 3/4 SIRS (ACN) | Spearman's Rho Correlation | 0.045 | 0.007 |
| DAF Steroid Use (ACN) | Spearman's Rho Correlation | 0.802 | 0.747 |
| DAF CVS Dysfunction (ACN) | Spearman's Rho Correlation | 0.395 | 0.070 |
| DAF Resp Dysfunction (ACN) | Spearman's Rho Correlation | 0.403 | 0.121 |
| DAF P/F <300 (ACN) | Spearman's Rho Correlation | 0.973 | 0.679 |
| DAF Ventilation (ACN) | Spearman's Rho Correlation | 0.219 | 0.090 |
| DAF CNS Dysfunction (ACN) | Spearman's Rho Correlation | 0.082 | 0.009 |
| DAF Coagulation (ACN) | Spearman's Rho Correlation | 0.067 | 0.017 |
| DAF INR >1.5 (ACN) | Spearman's Rho Correlation | 0.032 | 0.015 |
| DAF Renal Dysfunction (ACN) | Spearman's Rho Correlation | 0.070 | 0.008 |
| DAF Renal Support (ACN) | Spearman's Rho Correlation | 0.614 | 0.094 |
| DAF Hepatic Dysfunction (ACN) | Spearman's Rho Correlation | 0.041 | 0.007 |

Legend:
by Allele Copy Number (ACN), Kaplan-Meier (KM)

### Example 3

### Combination of EPCR and Protein C Polymorphisms

An interaction of novel haplotypes of protein C (protein C 4732 C) and EPCR(EPCR 4054 T) is associated with decreased survival and increased organ dysfunction in sirs, sepsis and septic shock

Subjects who had no copies of the EPCR risk allele (4054T) and no copies of the protein C risk allele (4732C) had the best 28 day survival and the least severity of organ dysfunction (protective-protective). Furthermore, subjects who had at least one copy of the EPCR risk allele (4054T) and at least one copy of the protein C risk allele (4732C) had the lowest survival and the greatest organ dysfunction (risk-risk). Finally, subjects who had either no copies of the EPCR risk allele (4054T) and at least one copy of the protein C risk allele (4732C) or who had at least one copy of the EPC-R risk allele (4054T) and no copies of the protein C risk allele (4732C) had intermediate survival and organ dysfunction. These findings are interesting and suggest that the interaction of SNP haplotypes of protein C and EPCR are important predictors of the outcomes of critically ill subjects who have SIRS.

Differences in the baseline characteristics of the subjects that were classified into groups 1, 2 and 3 are unlikely to explain the results. Particularly, since there were no differences in important predictors of outcome including age, APACHE II score, proportion of subjects who had sepsis at onset of the study and proportion of subjects who had septic shock at the onset of the study.

Previously it was not known whether interactions of risk alleles of protein C and risk alleles of EPCR were associated with altered outcomes in systemic inflammatory response syndrome (SIRS) or sepsis. We show that interactions of alleles of protein C and EPCR that are associated with increased risk of poor outcome (“risk alleles”) is associated with increased risk of death and organ dysfunction in systemic inflammatory response syndrome (SIRS), sepsis and septic shock.

Our study was based on an inception cohort of 487 critically ill Caucasian subjects who met at least 2/4 SIRS criteria. We defined subgroups who had sepsis (n=393) and who had septic shock (n=260). Outcomes were 28-day survival and severity of organ dysfunction by calculating days alive and free (DAF) of organ dysfunction (Brussels score: respiratory, cardiovascular, renal, coagulation, International Normalized Ratio for Partial Thromboplastin Time (INR)<1.5, hepatic, and neurological dysfunction and use of vasopressors, inotropic agents, and renal support by continuous renal replacement therapy or dialysis (renal support)). Haplotypes and clades of protein C and EPCR were determined by PHASE and MEGA 2 in Caucasians. Haplotype tag SNP's were selected that tagged each haplotype clade. Previously identified novel haplotypes with risk alleles of protein C (tagged by 4732 C) and EPCR (4054 T) associated with increased risk of death and organ dysfunction. Therefore, we classified subjects into 3 groups as having copies of protein C and EPCR risk alleles defined as follows:

Risk—Risk Group 1: defined subjects who had at least 1 copy of the risk allele of protein C 4732 C and at least I copy of the EPCR 4054 T.

Risk—Protective Group 2: defined subjects who had no risk alleles of protein C 4732 C and at last 1 copy of EPCR 4054 T or at least 1 copy of the protein C 4732 C and no copies of the EPCR 4054 T.

Protective—Protective Group 3: defined subjects who had no copies of the protein C 4732 C and no copies of the EPCR 4054 T (wild type).

| EPCR | SNP 4054 | Designation |
|---|---|---|
| | 4054T | Risk |
| | 4054C | Protective |

| ProC | SNP 4732 | Designation |
|---|---|---|
| | 4732C | Risk |
| | 4732T | Protective |

Associations of these 3 risk groups (Risk-Risk; Risk-Protective; Protective-Protective) with 28 day survival and with organ dysfunction as scored by days alive and free of organ dysfunction were tested for.

The organ dysfunction of subjects who had SIRS according to group is shown in Table 9. There was a steady increase in organ dysfunction (scored as lower days alive and free of organ dysfunction and support) from Protective-Protective, through Risk-Protective to Risk-Risk groups.

TABLE 9
Days alive and free (DAF) of use of vasopressors, coagulation (platelets)
dysfunction, INR, renal, cardiovascular dysfunction, hepatic dysfunction, SIRS 4 of
4, neurological (CNS) dysfunction and use of inotropic agents (inotropes) in 487
critically ill subjects who had Systemic Inflammatory Response Syndrome (SIRS)
according to group.

| PC 4732 | | | | | |
|---|---|---|---|---|---|
| C/EPCR | | | | | |
| 4054T | | | | | |
| Risk | DAF | DAF | | | |
| Group | Vasopressors | Coagulation | DAF INR | DAF Renal | DAF CVS |
| Risk-Risk | 16.1 ± 11.3 | 18 ± 11.2 | 17.6 ± 11.5 | 15.2 ± 12 | 14.5 ± 11.2 |
| Risk- | 18.5 ± 11.3 | 19.5 ± 11.3 | 19 ± 11.4 | 17.6 ± 11.6 | 16.1 ± 11.2 |
| Protective | | | | | |
| Protective- | 20.4 ± 10.4 | 21.5 ± 10 | 20 ± 10.5 | 19.5 ± 11.2 | 18.8 ± 10.6 |
| Protective | | | | | |
| P value | <0.003 | <0.06 | <0.05 | <0.10 | <0.018 |
| | Mean ± Standard | Mean ± Standard | Mean ± Standard | Mean ± Standard | Mean ± Standard |
| | Deviation | Deviation | Deviation | Deviation | Deviation |

| PC 4732 | | | | |
|---|---|---|---|---|
| C/EPCR | | | | |
| 4054T Risk | | DAF SIRS 4 | | |
| Group | DAF Hepatic | of 4 | DAF CNS | DAF Inotropes |
| Risk-Risk | 18.1 ± 11.3 | 17.3 ± 10.6 | 17.2 ± 11.5 | 18.8 ± 11.3 |
| Risk- | 19.8 ± 11.1 | 19.2 ± 10.5 | 19 ± 11 | 20.2 ± 10.7 |
| Protective | | | | |
| Protective- | 20.8 ± 11.1 | 21 ± 10 | 21 ± 10.4 | 22.1 ± 9.8 |
| Protective | | | | |
| P value | <0.06 | <0.004 | <0.11 | <0.034 |
| | Mean ± Standard Deviation | Mean ± Standard | Mean ± Standard Deviation | Mean ± Standard |
| | | Deviation | | Deviation |

*Significance for days alive and free of organ dysfunction tested by Spearman's rho statistic.

Subjects with SIRS in the Protective-Protective Group had 28 day survival of 73.7%, subjects in the Risk-Protective Group had 28 day survival of 67%, and subjects in the Risk-Risk Group had 28 day survival of 58.4% (p<0.02 by Chi-square; p<0.03 by Kaplan-Meier survival analysis over 28 days).

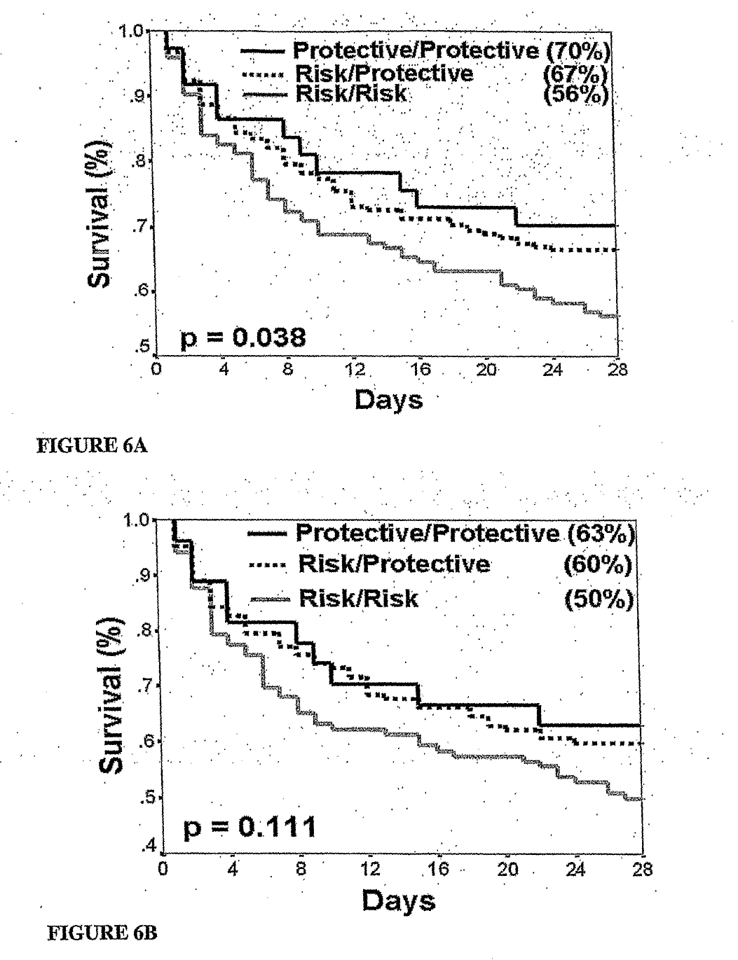

Subjects with sepsis (n=393) in the Protective-Protective Group had 28 day survival of 70.3%, subjects in the Risk-Protective Group had 28 day survival of 67%, and subjects in the Risk-Risk Group had 28 day survival of 56% (p<0.04 by Kaplan-Meier survival analysis over 28 days as shown in FIG. 6A).

Subjects with septic shock (n=260) in the Protective-Protective Group had 28 day survival of 63%, subjects in the Risk-Protective Group had 28 day survival of 60%, and subjects in the Risk-Risk Group had 28 day survival of 50% (Kaplan-Meier survival analysis over 28 days as shown in FIG. 6B).

The organ dysfunction of subjects who had sepsis according to group is shown in Table 10. There was a steady increase in organ dysfunction (scored as lower days alive and free of organ dysfunction and support) from Protective-Protective, through Risk-Protective to Risk-Risk groups.

TABLE 10
Days alive and free (DAF) of use of vasopressors, coagulation (platelets)
dysfunction, INR, renal, cardiovascular dysfunction, hepatic dysfunction, SIRS 4 of
4, neurological (CNS) dysfunction, use of inotropic agents (inotropes), and renal
support in 393 critically ill subjects who had Sepsis according to group.

| PC 4732 | | | | | |
|---|---|---|---|---|---|
| C/EPCR | | | | | |
| 4054T Risk | DAF | DAF | | | |
| Group | Vasopressors | Coagulation | DAF INR | DAF Renal | DAF CVS |
| Risk-Risk | 15.6 ± 11.1 | 17.7 ± 11.1 | 17.2 ± 11.4 | 14.7 ± 11.8 | 13.4 ± 11 |
| Risk- | 18.4 ± 11.1 | 19.8 ± 10.9 | 19 ± 11.2 | 17.6 ± 11.4 | 15.8 ± 11 |
| Protective | | | | | |
| Protective- | 19.3 ± 10.4 | 21.2 ± 1 | 19.7 ± 10.2 | 19.4 ± 11 | 17.6 ± 10.5 |
| Protective | | | | | |
| P value | <0.007 | <0.031 | <0.036 | <0.006 | <0.055 |
| | Mean ± Standard | Mean ± Standard | Mean ± Standard | Mean ± Standard | Mean ± Standard |
| | Deviation | Deviation | Deviation | Deviation | Deviation |

| PC | | | | |
|---|---|---|---|---|
| 4732 C/EPCR 4054T | | | | |
| Risk Group | DAF Hepatic | DAF SIRS 4 of 4 | DAF CNS | DAF Inotropes |
| Risk-Risk | 17.7 ± 11.2 | 16.9 ± 10.4 | 16.6 ± 11.4 | 18.5 ± 11.2 |
| Risk-Protective | 19.9 ± 10.9 | 19.1 ± 10.2 | 19.1 ± 10.7 | 20.4 ± 10.5 |
| Protective- | 20.8 ± 11 | 20.1 ± 10 | 20.3 ± 10.3 | 21.8 ± 10 |
| Protective | | | | |
| P value | <0.028 | <0.007 | <0.021 | <0.013 |
| | Mean ± | Mean ± | Mean ± | Mean ± |
| | Standard Deviation | Standard Deviation | Standard Deviation | Standard Deviation |

| PC 4732 C/EPCR 4054T Risk Group | DAF Renal Support |
|---|---|
| Risk-Risk | 16.1 ± 11.9 |
| Risk-Protective | 18.1 ± 11.8 |
| Protective-Protective | 17.8 ± 12.2 |
| P value | <0.09 |
| | Mean ± Standard Deviation |

Significance for days alive and free of organ dysfunction tested by Spearman's rho statistic.

There is an interaction between risk alleles of protein C (4732C) and EPCR (4054T) (as defined above) that is associated with increased risks of death and multiple organ dysfunctions in systemic inflammatory response syndrome (SIRS), sepsis and septic shock.

### Example 4

### Improved Response to Therapy with Activated Protein C (XIGRIS™)

Therapies for sepsis may include mechanical ventilation, support of circulation with vasopressors and inotropic agents, antibiotics, drainage of abscesses and surgery as appropriate. Activated protein C (APC or XIGRIS™ (when referring to APC as sold by Eli Lilly & Co., Indianapolis Ind.)) can improve survival of sepsis subjects. The PROWESS trial showed that XIGRIS™ decreased 28 day mortality from 31% (placebo) to 25% (APC/XIGRIS™—treated). XIGRIS™ was particularly effective in subjects at high risk of death for example as identified by having an APACHE II score greater than or equal to 25. XIGRIS™ has been approved for treatment of severe sepsis at increased risk of death. In some jurisdictions, the high risk of death is identified as having an APACHE II score greater than or equal to 25; in other jurisdictions high risk of death is identified as having 2 or more organ dysfunctions or having an APACHE II score greater than or equal to 25.

The severity of organ dysfunction that occurs in the setting of sepsis was also examined to determine whether XIGRIS™ reduces organ dysfunction in subjects who have sepsis and who have an at risk genotype of protein C or EPCR (protein C 4732 C and EPCR 4054 T).

### Example 4

### General Methods

Subject Inclusion Criteria

XIGRIS™—treated subjects were critically ill patients with severe sepsis, no XIGRIS™ contraindications and were treated with XIGRIS™. Whereas control subjects were critically ill patients who had severe sepsis who (at least 2 of 4 SIRS criteria, known or suspected infection, and APACHE II≧25), a platelet count>30,000, INR<3.0, bilirubin<20 mmol/L (i.e. no evidence of chronic hepatic dysfunction) and were not treated with XIGRIS™. Accordingly, the control group (untreated with XIGRIS™) is comparable to the XIGRIS™-treated group.

Genotyping

Protein C T4732C and EPCR T4054C were genotyped using the TaqMan™ assay (Applied Biosystems) as described above.

Clinical Phenotypes

The outcomes or clinical phenotypes were survival (28 day) and organ dysfunction scored as days alive and free of organ dysfunction according to Brussels score (as described above).

Statistical Analysis

Baseline characteristics age, gender, APACHE II, and percent surgical patients were recorded in all groups and compared. The 28 day survival rate (%) for patients who were protein C 4732 CC/CT were compared to patients who were protein C 4732 TT (wild type) with and without treatment with XIGRIS™. Kaplan-Meier 28 day survival curves and log rank test were determined to compare protein C 4732 CC/CT with and without XIGRIS™ treatment and also protein C 4732 TT with and without XIGRIS™ treatment.

Similarly, the 28 day survival rate (%) for patients who were EPCR 4054 TT were compared to patients who were EPCR 4054 CC/CT with and without treatment with XIGRIS™. Kaplan-Meier 28 day survival curves and log rank test were determined to compare EPCR 4054 TT with and without XIGRIS™ treatment and also EPCR 4054 CC/CT with and without XIGRIS™ treatment.

The 28 day survival rate (%) for patients who carried at least one copy of protein C 4732C and one copy of EPCR 4054 T (EPCR 4054 T) (risk-risk), patients who carried one copy of either protein C 4732C or EPCR 4054 (risk-protective) and patients who carried no copies of either protein C 4732C or EPCR 4054 (protective-protective) with and without treatment with XIGRIS™ were compared. Kaplan-Meier 28 day survival curves and log rank test were also determined to compare patients who carried at least one copy of protein C 4732C and one copy of EPCR 4054 T (EPCR 4054 T) (risk-risk) to patients who carried one copy of either protein C 4732C or EPCR 4054 (risk-protective) and patients who carried no copies of either protein C 4732C or EPCR 4054 (protective-protective).

Bootstrap simulations were performed for the prediction of survival for patients who were protein C 4732 CC/CT who were treated with XIGRIS™ vs. patients who were protein C 4732 CC/CT who were not treated with XIGRIS™. Bootstrap simulations were also performed for the prediction of survival for patients who were protein C 4732 TT who were treated with XIGRIS™ vs. patients who were protein C 4732 TT who were not treated with XIGRIS™.

Bootstrap simulations were performed for the prediction of survival for patients who were EPCR 4054TT who were treated with XIGRIS™ vs. patients who were EPCR 4054CC/CT who were not treated with XIGRIS™. Bootstrap simulations were performed for the prediction of survival for patients who were EPCR 4054CC/CT who were treated with XIGRIS™ vs. patients who were EPCR 4054CC/CT who were not treated with XIGRIS™.

Bootstrap simulations were performed for the prediction of survival for patients who carried at least one copy of protein C 4732C and one copy of EPCR 4054 T (EPCR 4054 T) (risk-risk), patients who carried one copy of either protein C 4732C or EPCR 4054 (risk-protective) and patients who carried no copies of either protein C 4732C or EPCR 4054 (protective-protective) who were treated with XIGRIS™ vs. patients who carried at least one copy of protein C 4732C and one copy of EPCR 4054 T (EPCR 4054 T) (risk-risk), patients who carried one copy of either protein C 4732C or EPCR 4054 (risk-protective) and patients who carried no copies of either protein C 4732C or EPCR 4054 (protective-protective)who were not treated with XIGRIS™.

Bootstrap simulation results were in all cases expressed as median and 95% confidence intervals. Study subjects were sampled 10,000 times with replacement using the sampling unit as the union of all subjects in the APC and control groups. For each replicate sample, a Cox proportional hazards (CPH) regression analysis was carried out, and a relative risk (APC vs. controls) was computed by exponentiating the CPH regression parameter. The 95% confidence interval for the relative risk was then obtained using the median value and the upper and lower 2.5% percentiles of the empirical distribution of the 10,000 relative risks.

In the Tables below abbreviations are used as indicated in the following list:
Surv: 28 day survival %, (number of patients).
ALI.DAF: Days alive and free of acute lung injury.
PRESS.DAF: Days alive and free of vasopressors.
INO.DAF: Days alive and free of inotropic agents.
MSIRS4.DAF: Days alive and free of 4/4 SIRS criteria.
CVS.DAF: Days alive and free of cardiovascular dysfunction.
RESP.DAF: Days alive and free of respiratory dysfunction.
CNS.DAF: Days alive and free of neurological dysfunction.
COAG.DAF: Days alive and free of coagulation dysfunction.
INR.DAF: Days alive and free of INR>1.5.
ACRF.DAF: Days alive and free of acute renal dysfunction.
ANYREN.DAF: Days alive and free of any renal dysfunction.
RENSUP.DAF: Days alive and free of renal support.
ACHBEP.DAP: Days alive and free of acute hepatic dysfunction.
ANYHEP.DAF: Days alive and free of any hepatic dysfunction.
PF300.DAF: Days alive and free of PaO2/FiO2 ratio<300
PRESS15.DAF: days alive and free of 15 units of vasopressors
PRESS5.DAF: days alive and free of 5 units of vasopressors
PRESS2.DAF: days alive and free of 2 units of vasopressors
PRESS.DAF: days alive and free of any vasopressors.

### Example 4A

### Protein C T4732C Improved Response Polymorphism and XIGRIS™ Treatment

Sample Size

There were 44 patients who were genotyped for protein C T4732C who were treated with XIGRIS™ and 226 control patients (not treated with XIGRIS™) who were genotyped for protein C T4732C. Among the XIGRIS™-treated patients (N=44), there were 19 patients who were protein C 4732 CC/CT and 25 patients who were protein C 4732 TT. Among the control patients (not treated with XIGRIS™) (N=226), there were 102 patients who were protein C 4732 CC/CT and 124 patients who were protein C 4732 TT.

TABLE 11
Baseline characteristics (Age, Gender, % Surgical, APACHE II) are shown
for patients who were treated with XIGRIS ™ and control patients (not treated with
XIGRIS ™) who were genotyped for protein C T4732C. (Data are shown as 25 percentile,
median, and 75 percentile)

| VARIABLE | Control (N = 226) | XIGRIS ™ (N = 44) | Test Statistic |
|---|---|---|---|
| AGE | 51.00/63.00/73.00 | 37.00/52.00/64.75 | F = 11.11, DF = 1.268, P < 0.001 |
| APACHEII | 27.00/29.00/33.75 | 23.00/30.50/36.25 | F = 0.81, DF = 1.268, P = 0.37 |
| SEX | 65%(147) | 57%(25) | Chi-square = 1.08, DF = 1, P = 0.299 |
| SURGICAL | 20%(45) | 27%(12) | Chisquare = 1.2, DF = 1, P = 0.274 |

The baseline characteristics of subjects who had severe sepsis and who were treated with XIGRIS™ are shown in Table 11. These subjects are typical of subjects who have severe sepsis with regards to age, sex and APACHE II score.

TABLE 12
28-day survival of patients treated with XIGRIS ™
and controls (patients not treated with XIGRIS ™)
by protein C T4732 C risk group.

| Row | GENOTYPE | Control | XIGRIS ™ | ALL |
|---|---|---|---|---|
| 1 | CC, CT | 45% (46/102) | 68% (13/19) | 49% (59/121) |
| 2 | TT | 56% (69/124) | 60% (15/25) | 56% (84/149) |
| 3 | ALL | 51% (115/226) | 64% (28/44) | 53% (143/270) |

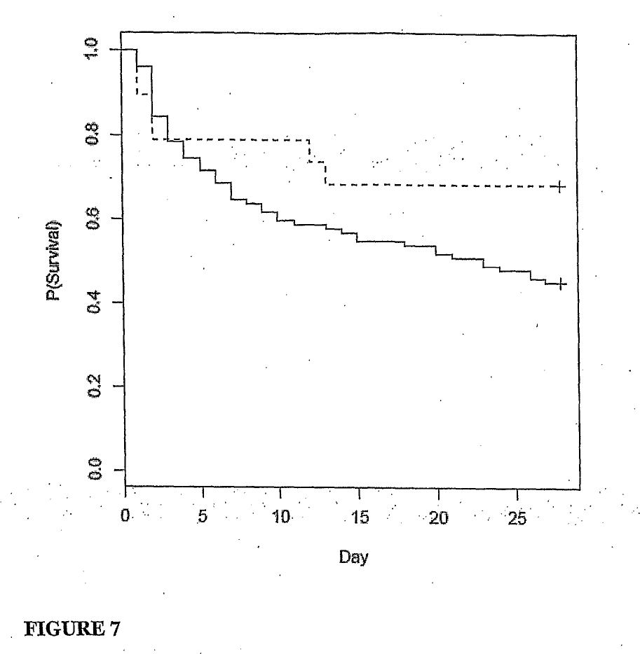

FIG. 7 shows Kaplan-Meier survival curves for patients who were protein C 4732 CC/CT and who were and who were not treated with XIGRIS™. The solid line indicates patients who were protein C 4732 CC/CT who were not treated with XIGRIS™ (i.e. control) and the dashed line indicates patients who were protein C 4732 CC/CT who were treated with XIGRIS™.

Statistical Testing by Cox Proportional Hazards

Treatment with XIGRIS™ coefficient=−0.654 for risk of death (i.e. XIGRIS™ decreased the risk of death), relative risk of death was 0.52 (of patients who were protein C 4732 CC/CT XIGRIS™-treated compared to patients who were protein C 4732 CC/CT not treated with XIGRIS™), overall p-value 0.097 for reduced risk of death when XIGRIS™ treatment is given to patients who were protein C 4732 CC/CT (high risk genotype). Accordingly, XIGRIS™ treatment increases survival (compared to no treatment) of patients who were protein C 4732 CT/CC.

Statistical Testing by Log Rank Test

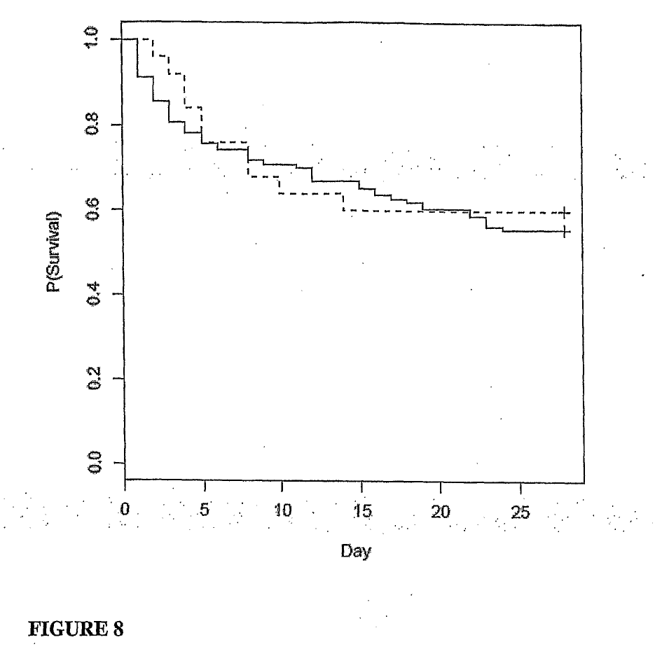

Treatment with XIGRIS™ increased survival in patients who carried the protein C 4732 C allele (CC/CT) compared to patients who carried the protein C 4732 C allele (CC/CT) who were not treated with XIGRIS™ (P-value=0.11). Thus, XIGRIS™ treatment increases survival rate over 28 days of patients who were protein C 4732 CC/CT (high risk genotype). Compare also to FIG. 8 in which XIGRIS™ treatment had virtually no effect on survival rate over 28 days in patients who were protein C 4732 TT (wild type genotype).

Bootstrap Analysis

Risk group (patients who carried the protein C 4732 C allele (CC/CT): Relative risk of death when treated with XIGRIS™ compared to not treated with XIGRIS™; median=0.5175, 95% confidence intervals 0.1589, 1.0558 respectively. This again demonstrates that XIGRIS™ treatment significantly increases survival rate over 28 days in patients who were protein C 4732 CC/CT (high risk genotype) when compared to patients who were protein C 4732 CT/CC who were not treated with XIGRIS™.

FIG. 8 shows Kaplan-Meier survival curves of patients who were protein C 4732 TT who were and were not treated with XIGRIS™. The solid line indicates patients who were protein C 4732 TT who were not treated with XIGRIS™ (i.e. control) and the dashed line indicates patients who were protein C 4732 TT who were treated with XIGRIS™.

Statistical Testing by Cox Proportional Hazards

Treatment with XIGRIS™ coefficient=−0.135 for reduced risk of death (i.e. XIGRIS™ did not decrease the risk of death) of patients who were protein C 4732 TT XIGRIS™—treated compared to patients who were protein C 4732 TT not treated with XIGRIS™, relative risk of death was 0.873, overall p-value 0.689 for reduced risk of death when XIGRIS™ treatment is given to patients who were protein C 4732 TT (wild type genotype). Thus, XIGRIS™ treatment had virtually no effect on survival rate over 28 days in patients who were protein C 4732 IT (wild type genotype).

Statistical Testing by Log-rank Test: Treatment with XIGRIS™ did not increase survival in patients who were protein C 4732 TT compared to patients who were protein C 4732 TT Who were not treated with XIGRIS™ (P value=0.983). Thus, XIGRIS™ treatment had virtually no effect on survival rate over 28 days in patients who were protein C 4732 TT (wild type genotype).

Bootstrap Analysis

Risk group (patients who were protein C 4732 TT: Relative risk of death when treated with XIGRIS™ compared to not treated with XIGRIS™; median=0.8742, 95% confidence intervals 0.3131, 1.4717 respectively. This again demonstrates that XIGRIS™ treatment had virtually no effect on survival rate over 28 days in patients who were protein C 4732 TT (wild type genotype).

TABLE 13
Organ Dysfunction Scored as Days Alive and Free (DAF)
of Organ Dysfunction (25% ile, median, 75% ile) of Patients
who were not treated with XIGRIS ™ according to protein
C allele (patients who were protein C 4732 CC/CT (Risk)
vs. patients who were wild type (WT) (protein C 4732 TT)).

| NO XIGRIS ™ | RISK (N = 102) | WT (N = 124) |
|---|---|---|
| SURV | 45% (46) | 56% (69) |
| ALI.DAF | 2.00/9.50/25.00 | 2.00/10.50/27.00 |
| PRESS.DAF | 1.25/14.00/25.00 | 2.00/20.50/26.00 |
| INO.DAF | 4.00/21.50/28.00 | 4.75/26.00/28.00 |
| MSIRS4.DAF | 3.0/12.5/25.0 | 5.0/23.0/27.0 |
| CVS.DAF | 1/8/2022 | 1/16/2025 |
| RESP.DAF | 0.0/3.0/18.0 | 0.0/6.5/22.0 |
| CNS.DAF | 2.0/11.5/26.0 | 3.0/22.0/27.0 |
| COAG.DAF | 4/15/2028 | 3/24/2028 |
| INR.DAF | 3.0/16.0/28.0 | 3.0/22.5/28.0 |
| ACRF.DAF | 2.0/9.5/26.0 | 2.0/16.0/28.0 |
| ANYREN.DAF | 0.00/4.00/19.75 | 1.00/11.00/27.00 |
| RENSUP.DAF | 1.25/6.00/25.75 | 2.00/16.00/28.00 |
| ACHEP.DAF | 4.00/16.50/28.00 | 5.75/28.00/28.00 |
| ANYHEP.DAF | 4.00/15.50/28.00 | 5.75/28.00/28.00 |

In general, patients who were not treated with XIGRIS™ who were protein C 4732 CC/CT had more organ dysfunction as indicated by fewer days alive and free of organ dysfunction compared to patients who were not treated who were protein C 4732 TT (wild type). This indicates that patients who were protein C 4732 CC/CT had greater risk of organ dysfunction compared to patients who were protein C 4732 TT in the absence of treatment with XIGRIS™.

TABLE 14
Organ Dysfunction Scored as Days Alive and Free (DAF)
of Organ Dysfunction (25% ile, median, 75% ile) of Patients
who were treated with XIGRIS ™according to protein
C allele (patients who were protein C 4732 CC/CT vs.
patients who were wild type (protein C 4732 TT)).

| XIGRIS ™ | RISK (N = 19) | WT (N = 25) |
|---|---|---|
| SURV | 68% (13) | 60% (15) |
| ALI.DAF | 1.00/12.00/24.00 | 3.00/6.00/24.00 |
| PRESS.DAF | 0.5/24.0/27.0 | 0.0/23.0/26.0 |
| INO.DAF | 9.50/28.00/28.00 | 8.00/28.00/28.00 |
| MSIRS4.DAF | 8.00/19.00/24.50 | 4.00/17.00/27.00 |
| CVS.DAF | 0.0/15.0/24.5 | 0.0/16.0/26.0 |
| RESP.DAF | 0.0/9.0/21.0 | 0.0/14.0/21.0 |
| CNS.DAF | 4.00/23.00/27.00 | 6.00/25.00/26.00 |
| COAG.DAF | 9.50/23.00/28.00 | 5.00/24.00/28.00 |
| INR.DAF | 5.5/28.0/28.0 | 5.0/27.0/28.0 |
| ACRF.DAF | 1.0/15.0/28.0 | 5.0/25.0/28.0 |
| ANYREN.DAF | 1.00/15.00/28.00 | 5.00/14.00/28.00 |
| RENSUP.DAF | 1/9/2028 | 2/15/2028 |
| ACHEP.DAF | 1.0/28.0/28.0 | 8.0/24.0/28.0 |
| ANYHEP.DAF | 1.00/28.00/28.00 | 5.00/23.00/28.00 |

In general, patients who were protein C 4732 CT/CC who were treated with XIGRIS™ had less organ dysfunction as indicated by greater days alive and free (DAF) of organ dysfunction. This indicates that the treatment with XIGRIS™ lessened the risk of organ dysfunction in the patients who had the high risk genotype (protein C 4732 CC/CT). Compare also to TABLE 13 to see that in the absence of XIGRIS™ treatment, patients who have the protein C 4732 CC/CT normally have increased risk of organ dysfunction.

TABLE 15
Organ Dysfunction Scored as Days Alive and Free (DAF) of Organ
Dysfunction (Median, 25% ile, 75% ile) of Patients who were not treated with
XIGRIS ™ and patients who were treated with
XIGRIS ™ according to protein C allele (patients who were protein
C 4732 CC/CT vs. patients who were wild type (protein C 4732 TT)).
PROC.T4732C, RISK

| | TREATMENT | | | | | |
|---|---|---|---|---|---|---|
| | NO XIGRIS ™ (N = 226) | | | XIGRIS ™ (N = 44) | | |
| | RISK | | | RISK | | |
| | (N = 102) | WT (N = 124) | Δ | (N = 19) | WT (N = 25) | Δ |
| GENOTYPE | A | B | A − B | C | D | C − D |
| ALI.DAF | 9.5 | 10.5 | −1 | 12 | 6 | 6 |
| PRESS.DAF | 14 | 20.5 | −6.5 | 24 | 23 | 1 |
| INO.DAF | 21.5 | 26 | −4.5 | 28 | 28 | 0 |
| MSIRS4.DAF | 12.5 | 23 | −10.5 | 19 | 17 | 2 |
| CVS.DAF | 8 | 16 | −8 | 15 | 16 | −1 |
| RESP.DAF | 3 | 6.5 | −3.5 | 9 | 14 | −5 |
| PF300.DAF | 0 | 0 | 0 | 0 | 1 | −1 |
| VENT.DAF | 1 | 2 | −1 | 7 | 14 | −7 |
| CNS.DAF | 11.5 | 22 | −10.5 | 23 | 25 | −2 |
| COAG.DAF | 15 | 24 | −9 | 23 | 24 | −1 |
| INR.DAF | 16 | 22.5 | −6.5 | 28 | 27 | 1 |
| ACRF.DAF | 9.5 | 16 | −6.5 | 15 | 25 | −10 |
| ANYREN.DAF | 4 | 11 | −7 | 15 | 14 | 1 |
| RENSUP.DAF | 6 | 16 | −10 | 9 | 15 | −6 |
| ACHEP.DAF | 16.5 | 28 | −11.5 | 28 | 24 | 4 |
| ANYHEP.DAF | 15.5 | 28 | −12.5 | 28 | 23 | 5 |

### Example 4B

### EPCR 4054T Improved Response Polymorphism and XIGRIS™ Treatment

Sample Size

There were 46 patients who were genotyped for EPCR T4054C who were treated with XIGRIS™ and 231 control patients (not treated with XIGRIS™) who were genotyped for EPCR T4054C. Among the XIGRIS™-treated patients (N=46), there were 19 patients who were EPCR 4054TT, 18 patients who were EPCR 4054CT and 9 patients who were EPCR 4054CC. Among the control patients (not treated with XIGRIS™) (N=231), there were 66 patients who were EPCR 4054TT, 114 patients who were EPCR 4054CT and 51 patients who were EPCR 4054CC.

TABLE 16
Baseline characteristics (age, gender, % surgical, APACHE II)
are shown for patients who were treated with XIGRIS ™
and control patients (not treated with XIGRIS ™) of
patients who were genotyped for EPCR T4054C.

| | | XIGRIS ™ | |
|---|---|---|---|
| VARIABLE | Control (N = 231) | (N = 46) | Test Statistic |
| AGE | 51.00/63.00/73.00 | 37.25/52.00/66.25 | F = 11, DF = 1.275, |
| | | | P = 0.00103 |
| APACHEII | 27.00/29.00/33.00 | 23.00/30.50/36.75 | F = 0.74, |
| | | | DF = 1.275, |
| | | | P = 0.391 |
| SEX | 64% (148) | 57% (26) | Chisquare = 0.94, |
| | | | DF = 1, |
| | | | P = 0.333 |
| SURGICAL | 20% (46) | 30% (14) | Chisquare = 2.5, |
| | | | DF = 1, |
| | | | P = 0.114 |

TABLE 17
Survival (28 day) of patients treated with XIGRIS ™
and control patients (not treated with XIGRIS ™) by
EPCR 4054TT, EPCR 4054CT, and EPCR 4054CC.

| Rows | GENOTYPE | CONTROL | XIGRIS ™ | ALL |
|---|---|---|---|---|
| 1 | CC | 49% (25/51) | 44% (18/44) | 48% (29/60) |
| 2 | CT | 55.26 (63/114) | 61% (11/18) | 56% (74/132) |
| 3 | TT | 47% (31/66) | 74% (14/19) | 53% (45/85) |
| | ALL | 52% (119/231) | 63% (29/46) | 53% (148/277) |

There was a statistically significant increase in survival of patients who were EPCR 4054TT who were treated with XIGRIS™ (74%) compared to patients who were EPCR 4054TT who were control (survival=47%)(not treated with XIGRIS™) (Row 3). In contrast, patients who were EPCR 4054CT and patients who were EPCR 4054CC had little change in survival when treated with XIGRIS™ compared to control patients who were not treated with XIGRIS™ (Rows 1 and 2).

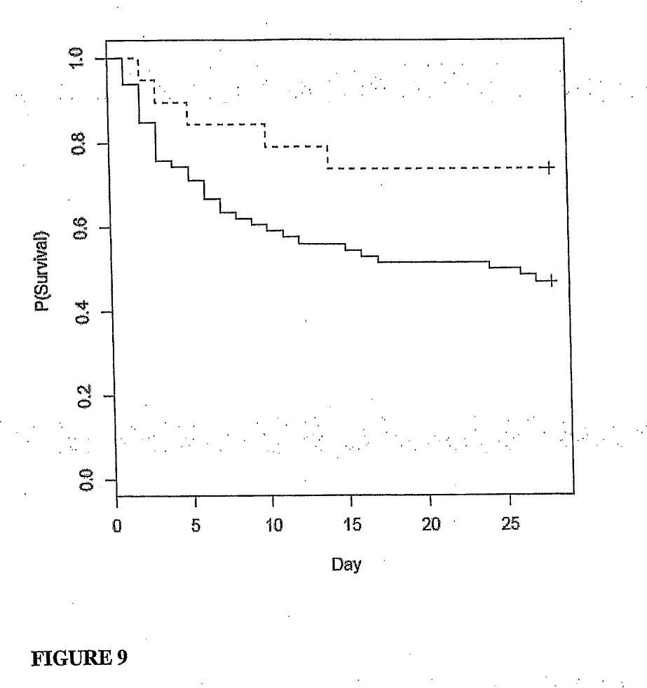

FIG. 9 shows Kaplan-Meier survival curves of patients who were EPCR 4054TT who were and who were not treated with XIGRIS™. The solid line indicates patients who were EPCR 4054TT who were not treated with XIGRIS™ (i.e. control) and the dashed line indicates patients who were EPCR 4054TT who were treated with XIGRIS™.

Statistical Testing by Cox Proportional Hazards

Treatment with XIGRIS™ coefficient=−0.895 for risk of death (i.e. XIGRIS™ decreased the risk of death), relative risk of death was 0.408 (of patients who were EPCR 20 4054TT XIGRIS™-treated compared to patients who were EPCR 4054TT not treated with XIGRIS™), overall p-value=0.037 for reduced risk of death when XIGRIS™ treatment is given to patients who were EPCR 4054TT (high risk genotype). This indicates that treatment with XIGRIS™ significantly increases survival (compared to no treatment) of patients who were EPCR 4054TT.

Statistical Testing by Log Rank Test

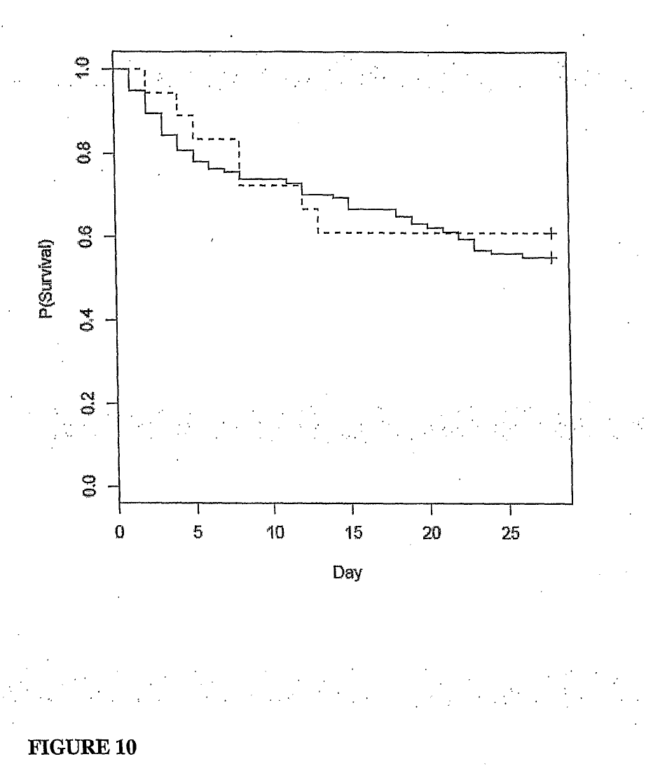

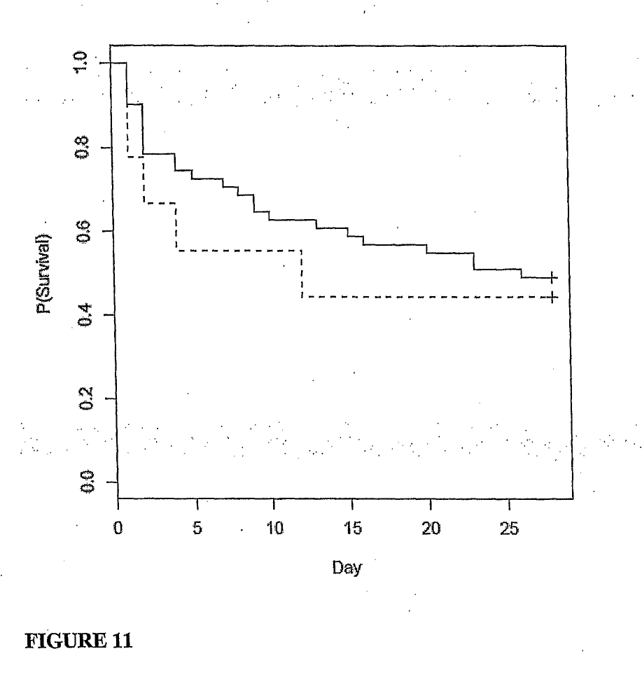

Treatment with XIGRIS™ increased survival in patients who were EPCR 4054TT compared to patients who were EPCR 4054TT who were not treated with XIGRIS™(P value=0.051). Thus, XIGRIS™ treatment significantly increased survival rate over 28 days in patients who were EPCR 4054TT. Compare also to FIGS. 10 and 11 in which XIGRIS™ treatment had virtually no effect on survival rate over 28 days in patients who were EPCR 4054CT and patients who were EPCR 4054CC.

Bootstrap Analysis

Risk group (patients who were EPCR 4054TT): Relative risk of-death when treated with XIGRIS™ compared to not treated with XIGRIS™; median=0.407, 95% confidence intervals 0.0725, 0.8431 respectively. This again demonstrates that XIGRIS™ treatment significantly increased survival rate over 28 days in patients who were EPCR 4054TT compared to patients who were EPCR 4054TT who were not treated with XIGRIS™.

FIG. 10 shows Kaplan-Meier survival curves of patients who were EPCR 4054CT who were and were not treated with XIGRIS™. The solid line indicates patients who were EPCR 4054CT who were not treated with XIGRIS™ (i.e. control) and the dashed line indicates patients who were EPCR 4054CT who were treated with XIGRIS™.

Statistical Testing by Cox Proportional Hazards

Treatment with XIGRIS™ coefficient=−0. 17 for reduced risk of death (i.e. XIGRIS™ did not decrease the risk of death) of patients who were EPCR 4054CT XIGRIS™-treated compared to patients who were EPCR 4054CT not treated with XIGRIS™, relative risk of death was 0.844, overall p-value 0.666 for reduced risk of death when XIGRIS™ treatment is given to patients who were EPCR 4054CT. Accordingly, XIGRIS™ treatment had virtually no effect on survival rate over 28 days in patients who were EPCR 4054CT.

Statistical Testing by Log Rank Test

Treatment with XIGRIS™ did not increase survival in patients who were EPCR 4054CT compared to patients who were EPCR 4054CT who were not treated with XIGRIS™ (P value=0.67). Accordingly, XIGRIS™ treatment had virtually no effect on survival rate over 28 days in patients who were EPCR 4054CT.

Bootstrap Analysis

Risk group (patients who were EPCR 4054CT: Relative risk of death when treated with XIGRIS™ compared to not treated, with XIGRIS™; median=0.842, 95% confidence intervals 0.331, 1.6212 respectively. This again demonstrates that XIGRIS™ treatment had virtually no effect on survival rate over 28 days in patients who were EPCR 4054CT.

FIG. 11 shows Kaplan-Meier survival curves of patients who were EPCR 4054CC who, were and were not treated with XIGRIS™ The solid line indicates patients who were EPCR 4054CC who were not treated with XIGRIS™ (i.e. control) and the dashed line indicates patients who were EPCR 4054CC who were treated with XIGRIS™.

Statistical Testing by Cox Proportional Hazards

Treatment with XIGRIS™ coefficient=0.254 for reduced risk of death (i.e. XIGRIS™ did not decrease the risk of death) of patients who were EPCR 4054CC XIGRIS™-treated compared to patients who were EPCR 4054CC not treated with XIGRIS™, relative risk of death was 1.289, overall p-value 0.61 for reduced risk of death when XIGRIS™ treatment is given to patients who were EPCR 4054CC. Accordingly, XIGRIS™ treatment had virtually no effect on survival rate over 28 days in patients who were EPCR 4054CC.

Statistical Testing by Log Rank Test

Treatment with XIGRIS™ did not increase survival in patients who were EPCR 4054CC compared to patients who were EPCR 40540C who were not treated with XIGRIS™ (P value=0.617). Accordingly, XIGRIS™ treatment had virtually no effect on survival rate over 28 days in patients who were EPCR 4054CC.

Bootstrap Analysis

Risk group (patients who were EPCR 4054CC: Relative risk of death when treated with XIGRIS™ compared to not treated with XIGRIS™; median=1.274, 95% confidence intervals 0.439, 2.842 respectively. This again demonstrates that XIGRIS™ treatment had virtually no effect on survival rate over 28 days in patients who were EPCR 4054CC.

TABLE 18
Organ Dysfunction Scored as Days Alive and Free (DAF)
of Organ Dysfunction (Median, 25% ile, 75% ile) of Patients
who were not treated with XIGRIS ™ according to
Endothelial Protein C Receptor T4054C (patients who were
Endothelial Protein C Receptor 4054TT (Risk) and patients
who were Endothelial Protein C Receptor 4054CT and patients
who were Endothelial Protein C Receptor 4054CC (Other).

| | | | Combined |
|---|---|---|---|
| NO XIGRIS ™ | RISK (N = 180) | OTHER (N = 51) | (N = 231) |
| SURV | 52% (94) | 49% (25) | 52% (119) |
| ALI.DAF | 2.00/13.50/27.25 | 1.00/5.00/26.00 | 2.00/10.00/26.75 |
| PRESS.DAF | 2.0/19.0/26.0 | 1.5/15.0/25.0 | 2.0/18.0/26.0 |
| INO.DAF | 4.75/25.00/28.00 | 4.00/26.00/28.00 | 4.00/24.50/28.00 |
| MSIRS4.DAF | 3.75/20.00/27.00 | 3.50/15.00/27.00 | 3.00/19.00/27.00 |
| CVS.DAF | 1.0/14.0/24.0 | 1.0/12.0/24.5 | 1.0/13.0/24.0 |
| RESP.DAF | 0.0/7.5/21.0 | 0.0/1.0/18.5 | 0.0/3.5/20.0 |
| CNS.DAF | 3/17/2027 | 2/19/2026 | 3/16/2027 |
| COAG.DAF | 4.0/23.5/28.0 | 3.0/17.0/28.0 | 4.0/21.0/28.0 |
| INR.DAF | 3.0/22.0/28.0 | 2.5/17.0/27.5 | 3.0/21.0/28.0 |
| ACRF.DAF | 2.0/13.0/27.0 | 2.0/13.0/28.0 | 2.0/12.5/27.0 |
| ANYREN.- | 0.0/8.5/26.0 | 1.0/8.0/26.5 | 0.0/7.5/26.0 |
| DAF | | | |
| RENSUP.DAF | 2.0/13.0/28.0 | 1.0/13.0/28.0 | 2.0/12.5/28.0 |
| ACHEP.DAF | 5.0/27.0/28.0 | 4.5/23.0/28.0 | 5.0/25.0/28.0 |
| ANYHEP.DAF | 5.0/27.0/28.0 | 4.5/23.0/28.0 | 5.0/25.0/28.0 |

In general, patients who were not treated with XIGRIS™ who were EPCR 4054TT had more organ dysfunction as indicated by fewer days alive and free of organ dysfunction compared to patients who were not treated who were EPCR 4054CT/CC. This indicates that the EPCR 4054TT is a risk for organ dysfunction in the absence of treatment with XIGRIS™.

TABLE 19
Organ Dysfunction Scored as Days Alive and Free (DAF)
of Organ Dysfunction (25% Quartile/Median/75% Quartile)
of Patients who were treated with XIGRIS ™ according
to EPCR T4054C (patients who were EPCR 4054TT
(risk) vs. patients who were 4054CT/CC (other)).

| | | | Combined |
|---|---|---|---|
| XIGRIS ™ | RISK (N = 37) | OTHER (N = 9) | (N = 46) |
| SURV | 68% (25) | 44% (4) | 63% (29) |
| ALI.DAF | 3.00/9.00/24.00 | 1.00/1.00/17.00 | 2.00/8.50/23.25 |
| PRESS.DAF | 4/24/2027 | 0/0/25 | 0/23/26 |
| INO.DAF | 10.00/28.00/28.00 | 1.00/12.00/28.00 | 7.75/27.00/28.00 |
| MSIRS4.DAF | 7.00/22.00/27.00 | 0.00/8.00/19.00 | 6.25/17.50/26.00 |
| CVS.DAF | 4.0/20.0/26.0 | 0.0/0.0/22.0 | 0.0/15.5/25.0 |
| RESP.DAF | 1.0/17.0/22.0 | 0.0/0.0/14.0 | 0.0/7.5/21.0 |
| CNS.DAF | 6.00/25.00/27.00 | 2.00/11.00/23.00 | 4.75/23.00/26.25 |
| COAG.DAF | 8.00/27.00/28.00 | 2.00/7.00/28.00 | 5.75/21.50/28.00 |
| INR.DAF | 6/28/2028 | 1/5/2027 | 5/27/2028 |
| ACRF.DAF | 5.0/25.0/28.0 | 1.0/4.0/28.0 | 2.5/17.0/28.0 |
| ANYREN.- | 5.00/18.00/28.00 | 1.00/4.00/28.00 | 1.75/14.00/28.00 |
| DAF | | | |
| RENSUP.DAF | 2/15/2028 | 1/6/2028 | 1/12/2028 |
| ACHEP.DAF | 8.0/27.0/28.0 | 1.0/4.0/28.0 | 4.0/26.5/28.0 |
| ANYHEP.DAF | 4.00/26.00/28.00 | 1.00/4.00/28.00 | 2.75/25.00/28.00 |

In general, patients who were treated with XIGRIS™ had less organ dysfunction as indicated by greater days alive and free (DAF) of organ dysfunction. It can be seen that XIGRIS™ treatment of patients who were EPCR 4054TT have higher survival and more days alive and free of organ dysfunction than patients who were 4054CT/CC (other). Accordingly, treatment with XIGRIS™ lessened the risk of organ dysfunction in the patients who had the risk genotype (EPCR 4054TT). As compared to TABLE 18 in the absence of XIGRIS™ treatment, patients who have the EPCR 4054TT normally have increased risk of organ dysfunction.

TABLE 20
Organ Dysfunction Scored as Days Alive and Free (DAF) of Organ
Dysfunction (Median, 25% ile, 75% ile) of Patients who were not treated with
XIGRIS ™ and patients who were treated with
XIGRIS ™ according to protein C allele (patients who were
protein C 4732 CC/CT vs. patients who were wild type (protein C 4732 TT).

| | TREATMENT | | | | | |
|---|---|---|---|---|---|---|
| | NO XIGRIS ™ (N = 231) | | | | | |
| | RISK | | | XIGRIS ™ (N = 46) | | |
| | (N = 180) | OTHER (N = 51) | Δ | RISK | OTHER | Δ |
| GENOTYPE | A | B | A − B | (N = 37) | (N = 9) | C − D |
| SURV (%) | 52 | 49 | +3 | 68 | 44 | 24 |
| ALI.DAF | 13.5 | 5 | +8.5 | 9 | 1 | +8 |
| PRESS.DAF | 19 | 15 | 4 | 24 | 0 | +24 |
| INO.DAF | 25 | 26 | −1 | 28 | 12 | +16 |
| MSIRS4.DAF | 20 | 15 | +5 | 22 | 8 | +14 |
| CVS.DAF | 14 | 1 | +13 | 20 | 0 | +20 |
| RESP.DAF | 7.5 | 1 | +6.5 | 17 | 0 | +17 |
| CNS.DAF | 17 | 19 | −2 | 25 | 11 | +14 |
| COAG.DAF | 23.5 | 17 | +6.5 | 27 | 7 | +20 |
| INR.DAF | 22 | 17 | +5 | 28 | 5 | +23 |
| ACRF.DAF | 13 | 13 | 0 | 25 | 4 | +21 |
| ANYREN.DAF | 8.5 | 8 | +0.5 | 18 | 4 | +14 |
| RENSUP.DAF | 13 | 13 | 0 | 15 | 6 | +9 |
| ACHEP.DAF | 27 | 23 | +4 | 27 | 4 | +23 |
| ANYHEP.DAF | 27 | 23 | +4 | 26 | 4 | +22 |

Patients not treated with XIGRIS™ (2^{nd}, 3^{rd} and 4^{th} columns), the survival and organ dysfunction scored as days alive and free of organ dysfunction of patients who have protein C 4732 CC/CT is compared to patients who are protein C 4732 TT by subtracting the survival and organ dysfunction scored as days alive and free of organ dysfunction of protein C 4732 CC/CT (marked A) from the survival and organ dysfunction scored as days alive and free of organ dysfunction of protein C 4732 TT (marked B) shown in the fourth column (marked A-B). Patients not treated with XIGRIS™, patients who have protein C 4732 CC/CT do consistently worse lower survival and fewer days alive and free of organ dysfunction) than patients who are protein C 4732 TT.

Patients treated with XIGRIS™ (5^{th}, 6^{th} and 7^{th} columns), the survival and organ dysfunction scored as days alive and free of organ dysfunction of patients who have protein C 4732 CC/CT is compared to patients who are protein C 4732 TT by subtracting the survival and organ dysfunction scored as days alive and free of organ dysfunction of protein C 4732 CC/CT (marked C) from the survival and organ-dysfunction scored as days alive and free of organ dysfunction of protein C 4732 TT (marked D) shown in the seventh column (marked C-D). It can be seen that in patients who are treated with XIGRIS™, patients who have protein C 4732 CC/CT do consistently better as shown by higher survival and more days alive and free of organ dysfunction) than patients who are protein C 4732 TT. This emphasizes the beneficial effects of XIGRIS™ in patients who have protein C 4732 CC/CT.

### Example 4C

### Protein C 4732C and EPCR 4054T Improved Response Polymorphism and XIGRIS™ Treatment

Sample Size

There were 13 patients who carried at least one copy of protein C 4732C and one copy of EPCR 4054 T (risk-risk) who were treated with XIGRIS™ and 78 control patients (not treated with XIGRIS™) who carried at least one copy of protein C 4732C and one copy of EPCR 4054 T (risk-risk).

There were 28 patients who carried one copy of either protein C 4732C or EPCR 4054 (risk-protective) who were treated with XIGRIS™ and 120 control patients (not treated with XIGRIS™) who carried one copy of either protein C 4732C or EPCR 4054 (risk-protective).

There were 3 patients who carried no copies of either protein C 4732C or EPCR 4054 (protective-protective) who were treated with XIGRIS™ and 26 control patients (not treated with XIGRIS™) who carried no copies of either protein C 4732C or EPCR 4054 (protective-protective).

Due to the relatively small sample size of patients who carried no copies of either protein C 4732C or EPCR 4054 (protective-protective), the risk-protective and the protective-protective groups were combined into a group called “other” for comparison to the patients who carried at least one copy of protein C 4732C and one copy of EPCR 4054 T (risk-risk).

TABLE 21
Baseline characteristics (age, gender, % surgical, APACHE II)
are for patients who were treated with XIGRIS ™ and
control patients (not treated with XIGRIS ™) of patients
who were genotyped for the combined genotype classified as
patients who carried at least one copy of protein C 4732C and
one copy of EPCR 4054 T (risk - risk), patients who carried one
copy of either protein C 4732C or EPCR 4054 (risk - protective)
and patients who carried no copies of either protein C 4732C
or EPCR 4054 (protective - protective) who were genotyped for
protein C T4732 C and for EPCR T4054C.

| | CONTROL | XIGRIS ™ | |
|---|---|---|---|
| VARIABLE | (N = 224) | (N = 44) | Test Statistic |
| AGE | 51.00/63.00/73.00 | 37.00/52.00/64.75 | F = 10.91, |
| | | | DF = 1.266, |
| | | | P = 0.00109 |
| APACHEII | 27.00/29.00/34.00 | 23.00/30.50/36.25 | F = 0.81, |
| | | | DF = 1.266, |
| | | | P = 0.369 |
| GENDER | 65% (145) | 57% (25) | Chisquare = 0.99, |
| | | | DF = 1, |
| | | | P = 0.319 |
| SURGICAL | 20% (44) | 27% (12) | Chisquare = 1.3, |
| | | | DF = 1, |
| | | | P = 0.255 |

TABLE 22
Survival (28-day) of patients treated with XIGRIS ™ and
control patients (not treated with XIGRIS ™) of patients who
were classified as patients who carried at least one copy of protein
C 4732C and one copy of EPCR 4054 T (risk - risk) compared to
patients who carried one copy of either protein C 4732C or EPCR
4054 (risk - protective) and patients who carried no copies of either
protein C 4732C or EPCR 4054 (protective - protective) pooled
and called Other.

| GENOTYPE | CONTROL | XIGRIS ™ | ALL |
|---|---|---|---|
| RISK-RISK | 49% (38/78) | 85% (11/13) | 54% (49/91) |
| Other | 52% (76/146) | 55% (17/31) | 53% (93/177) |
| ALL | 51% (114/224) | 64% (28/44) | 53% (142/268) |

There was a substantial increase in survival of patients who carried at least one copy of protein C 4732C and one copy of EPCR 4054 T (risk-risk) who were treated with XIGRIS™ (85%) compared to patients who carried at least one copy of protein C 4732C and one copy of EPCR 4054 T (risk-risk) who were control (not treated with XIGRIS™)(survival 49%).

In contrast, patients who carried one copy of either protein C 4732C or EPCR 4054 (risk-protective) and patients who carried no copies of either protein C 4732C or EPCR 4054 (protective-protective) (pooled and called other) had little change in survival when treated with XIGRIS™ compared to control patients who were not treated with XIGRIS™.

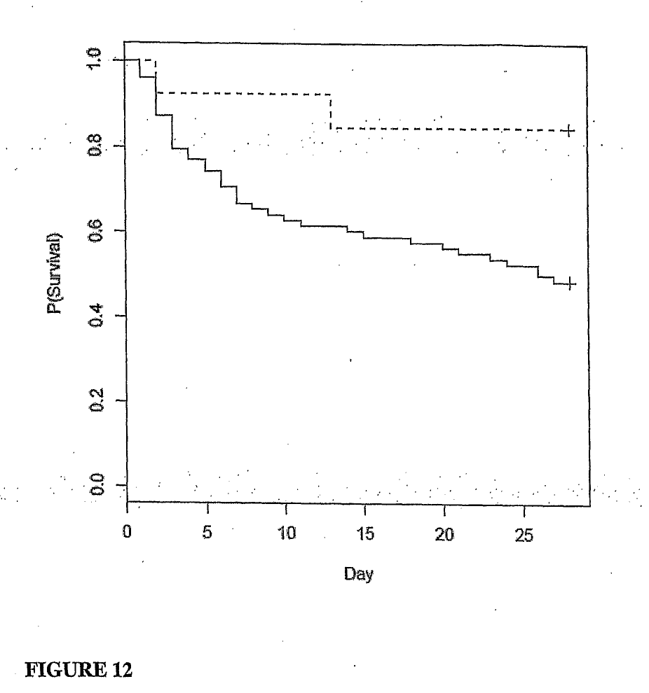

FIG. 12 shows Kaplan-Meier survival curves of patients who were classified as patients who carried at least one copy of protein C 4732C and one copy of EPCR 4054 T (risk-risk) who were and who were not treated with XIGRIS™. The solid line indicates patients who were classified as patients who carried at least one copy of protein C 4732C and one copy of EPCR 4054 T (risk-risk) not treated with XIGRIS™ (i.e. control) and the dashed line indicates patients who were classified as patients who carried at least one copy of protein C 4732C and one copy of EPCR 4054 T risk-risk) who were treated with XIGRIS™.

Statistical Testing by Cox Proportional Hazards

Treatment with XIGRIS™ coefficient=−1.436 for risk of death (i.e. XIGRIS™ decreased the risk of death), relative risk of death was 0.238 (of patients who were classified as patients who carried at least one copy of protein C 4732C and one copy of EPCR 4054 T (risk-risk) XIGRIS™-treated compared to patients who were classified as patients who carried at least one copy of protein C 4732C and one copy of EPCR 4054 T (risk-risk) not treated with XIGRIS™), overall p-value 0.013 for reduced risk of death when XIGRIS™ treatment is given to patients who were classified as patients who carried at least one copy of protein C 4732C and one copy of EPCR 4054 T (risk-risk). Suggesting that XIGRIS™ treatment significantly increases survival (compared to no treatment) of patients who carried at least one copy of protein C 4732C and one copy of EPCR 4054 T (risk-risk).

Statistical Testing by Log Rank Test

Treatment with XIGRIS™ increased survival in patients who were classified as patients who carried at least one copy of protein C 4732C and one copy of EPCR 4054 T (risk-risk) compared to patients who were classified as patients who carried at least one copy of protein C 4732C and one copy of EPCR 4054 T (risk-risk) who were not treated with XIGRIS™ (P value=0.03). Accordingly, XIGRIS™ treatment had a substantial effect on survival rate over 28 days in patients who were classified as patients who carried at least one copy of protein C 4732C and one copy of EPCR 4054 T (risk-risk).

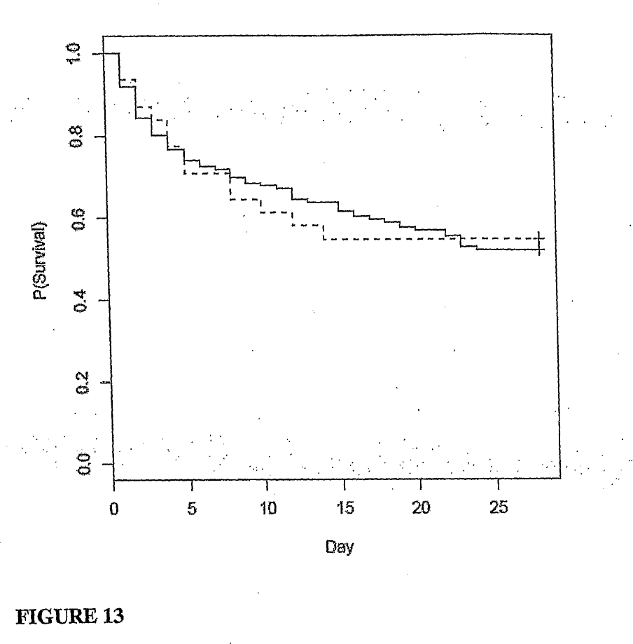

Compare also to FIG. 13 in which XIGRIS™ treatment had virtually no effect on survival rate over 28 days patients who carried one copy of either protein C 4732C or EPCR 4054T (risk-protective) or who carried no copies of either protein C 4732C or EPCR 4054T (protective-protective).

Bootstrap Analysis

Risk group (patients who were classified as patients who carried at least one copy of protein C 4732C and one copy of EPCR 4054 T (risk-risk): Relative risk of death when treated with XIGRIS™ compared to not treated with XIGRIS™; median 0.2369, 95% confidence intervals 0.00, 664 respectively. This again demonstrates that XIGRIS™ treatment significantly increases survival rate over 28 days of patients who were classified as patients who carried at least one copy of protein C 4732C and one copy of EPCR 4054 T (risk-risk) compared to patients who carried at least one copy of protein C 4732C and one copy of EPCR 4054 T (risk-risk) who were not treated with XIGRIS™.

FIG. 13 shows Kaplan-Meier survival curves of patients who carried one copy of either protein C 4732C or EPCR 4054T (risk-protective) and the protective-protective groups combined into a group called “other” who were and were not treated with XIGRIS™. The solid line indicates patients who carried one copy of either protein C 4732C or EPCR 4054T(risk-protective) and the protective-protective groups combined into a group called “other” who were not treated with XIGRIS™ (i.e. control) and the dashed line indicates patients who carried one copy of either protein C 4732C or EPCR 4054T (risk-protective) and the protective-protective groups combined into a group called “other” who were treated with XIGRIS™.

Statistical Testing by Cox Proportional Hazards

Treatment with XIGRIS™ coefficient=−0.043 for reduced risk of death (i.e. XIGRIS™ did not decrease the risk of death) of patients who carried one copy of either protein C 4732C or EPCR 4054 (risk-protective) and the protective-protective groups combined into a group called “other” XIGRIS™-treated compared to patients who carried one copy of either protein C 4732C or EPCR 4054 (risk-protective) and the protective-protective groups combined into a group called “other” not treated with XIGRIS™, relative risk of death was 0.958, overall p-value 0.88 for reduced risk of death when XIGRIS™ treatment is given to patients who carried one copy of either protein C 4732C or EPCR 4054 (risk-protective) and the protective-protective groups combined into a group called “other”. Thus, XIGRIS™ treatment had virtually no effect on survival rate over 28 days in patients who carried one copy of either protein C 4732C or EPCR 4054 (risk-protective) and the protective-protective groups combined into a group called “other”.

Statistical Testing by Log Rank Test

Treatment with XIGRIS™ did not increase survival in patients who carried one copy of either protein C 4732C or EPCR 4054 (risk-protective) and the protective-protective groups combined into a group called “other” compared to patients who carried one copy of either protein C 4732C or EPCR 4054 (risk-protective) and the protective-protective groups combined into a group called “other” who were not treated with XIGRIS™ (P value=0.881). Thus, XIGRIS™ treatment had virtually no effect on survival rate over 28 days in patients who carried one copy of either protein C 4732C or EPCR 4054 (risk-protective) and the protective-protective groups combined into a group called “other”.

Bootstrap Analysis

Risk group (patients who carried one copy of either protein C 4732C or EPCR 4054 (risk-protective) and the protective-protective groups combined into a group called “other”). Relative risk of death when treated with XIGRIS™ compared to not treated with XIGRIS™; median 0.952, 95% confidence intervals 0.497, 1.519 respectively. This again demonstrates that XIGRIS™ treatment had virtually no effect on survival rate over 28 days in patients who carried one copy of either protein C 4732C or EPCR 4054 (risk-protective) and the protective-protective groups combined into the “other” group.

TABLE 23
Organ Dysfunction Scored as Days Alive and Free (DAF) of
Organ Dysfunction (Median, 25% ile, 75% ile) of Patients who
were not treated with XIGRIS ™ according to protein
C allele (patients who carried at least one copy of protein
C 4732C and one copy of EPCR 4054 T (risk - risk) compared
to patients who carried one copy of either protein C 4732C or
EPCR 4054 (risk - protective) and the protective - protective
groups combined into a group called “other”.

| NO XIGRIS ™ | RISK-RISK (N = 78) | OTHER (N = 146) |
|---|---|---|
| SURV | 49% (38) | 52% (76) |
| ALI.DAF | 2.00/11.50/23.75 | 2.00/9.50/27.00 |
| PRESS.DAF | 2.0/18.0/25.0 | 2.0/18.5/26.0 |
| INO.DAF | 4.25/25.00/28.00 | 4.00/24.50/28.00 |
| MSIRS4.DAF | 3.00/16.50/24.75 | 4.00/19.50/27.00 |
| CVS.DAF | 0.25/11.00/22.75 | 1.00/13.50/24.00 |
| RESP.DAF | 0.00/5.50/19.75 | 0.00/3.00/20.75 |
| CNS.DAF | 3.0/14.0/26.0 | 3.0/20.5/27.0 |
| COAG.DAF | 4.00/20.00/28.00 | 3.25/22.50/28.00 |
| INR.DAF | 4.25/21.00/28.00 | 3.00/20.00/28.00 |
| ACRF.DAF | 2.00/11.00/26.75 | 2.00/13.00/28.00 |
| ANYREN.DAF | 0.0/5.5/23.0 | 1.0/8.5/27.0 |
| RENSUP.DAF | 2.0/6.5/28.0 | 2.0/14.5/28.0 |
| ACHEP.DAF | 4.25/20.50/28.00 | 5.00/28.00/28.00 |
| ANYHEP.DAF | 4.0/18.5/28.0 | 5.0/28.0/28.0 |

In general, patients who were not treated with XIGRIS™ who carried at least one copy of protein C 4732C and one copy of EPCR 4054 T (risk-risk) had more organ dysfunction as indicated by fewer days alive and free of organ dysfunction compared to patients who carried one copy of either protein C 4732C or 18PCR 4054 (risk-protective) and the protective-protective groups combined into a group called “other”.

This indicates that one copy of protein C 4732C and one copy of EPCR 4054 T (risk-risk) is a risk for organ dysfunction in the absence of treatment with XIGRIS™.

TABLE 24
Organ Dysfunction Scored as Days Alive and Free (DAF) of Organ
Dysfunction (Median, 25% ile, 75% ile) of Patients who were
treated with XIGRIS ™ according to patients who carried at
least one copy of protein C 4732C and one copy of EPCR 4054 T
(risk-risk) compared to patients who carried one copy of either
protein C 4732C or EPCR 4054 (risk-protective) and the
protective-protective groups combined into a group called “other”.

| XIGRIS ™ | RISK-RISK (N = 13) | OTHER (N = 31) |
|---|---|---|
| SURV | 85% (11) | 55% (17) |
| ALI.DAF | 4.00/19.00/25.00 | 2.00/5.00/22.00 |
| PRESS.DAF | 17/24/27 | 0/22/26 |
| INO.DAF | 25.00/28.00/28.00 | 3.50/26.00/28.00 |
| MSIRS4.DAF | 16.00/22.00/25.00 | 4.00/14.00/26.50 |
| CVS.DAF | 11.0/22.0/25.0 | 0.0/15.0/25.5 |
| RESP.DAF | 2.0/17.0/22.0 | 0.0/4.0/20.5 |
| CNS.DAF | 19.00/24.00/27.00 | 4.50/23.00/25.50 |
| COAG.DAF | 20.00/28.00/28.00 | 3.50/14.00/28.00 |
| INR.DAF | 26.0/28.0/28.0 | 4.5/26.0/28.0 |
| ACRF.DAF | 12.0/26.0/28.0 | 2.0/14.0/28.0 |
| ANYREN.DAF | 2.00/19.00/28.00 | 2.00/14.00/28.00 |
| RENSUP.DAF | 1/25/2028 | 2/14/2028 |
| ACHEP.DAF | 4.0/28.0/28.0 | 4.5/23.0/28.0 |
| ANYHEP.DAF | 4.00/28.00/28.00 | 3.50/19.00/28.00 |

In general, patients who were treated with XIGRIS™ had less organ dysfunction as indicated by greater days alive and, free (DAF) of organ dysfunction. This indicates that the treatment with XIGRIS™ lessened the risk of organ dysfunction in the patients who had the high risk genotype (i.e. patients who carried at least one copy of protein C 4732C and one copy of EPCR 4054 T (risk-risk).

As compared to TABLE 23 in the absence of XIGRIS™ treatment, patients who carried at least one copy of protein C 4732C and one copy of EPCR 4054 T (risk-risk) normally have increased risk of organ dysfunction

TABLE 25
Organ Dysfunction Scored as Days Alive and Free (DAF) of Organ
Dysfunction (Median, 25% ile, 75% ile) of Patients who were not treated with
XIGRIS ™ and Patients who were treated with XIGRIS ™ according to patients who
carried at least one copy of protein C 4732C and one copy of EPCR 4054 T (risk-risk)
compared to patients who carried one copy of either protein C 4732C or EPCR 4054 (risk-
protective) and the protective-protective groups combined into a group called “other”.

| | NO XIGRIS ™ (N = 224) | | | XIGRIS ™ (N = 44) | | |
|---|---|---|---|---|---|---|
| | RISK-RISK | OTHER | | RISK-RISK | OTHER | |
| TREATMENT | (N = 78) | (N = 146) | Δ | (N = 13) | (N = 31) | Δ |
| GENOTYPE | A | B | A − B | C | D | C − D |
| ALI.DAF | 11.5 | 9.5 | 2 | 19 | 5 | 14 |
| PRESS.DAF | 18 | 18.5 | −0.5 | 24 | 22 | 2 |
| PRESS2.DAF | 18 | 18.5 | −0.5 | 25 | 22 | 3 |
| PRESS5.DAF | 19 | 19.5 | −0.5 | 26 | 22 | 4 |
| PRESS15.DAF | 21 | 23 | −2 | 27 | 25 | 2 |
| INO.DAF | 25 | 24.5 | 0.5 | 28 | 26 | 2 |
| MSIRS4.DAF | 16.5 | 19.5 | −3 | 22 | 14 | 8 |
| CVS.DAF | 11 | 13.5 | −2.5 | 22 | 15 | 7 |
| RESP.DAF | 5.5 | 3 | 2.5 | 17 | 4 | 13 |
| CNS.DAF | 14 | 20.5 | −6.5 | 24 | 23 | 1 |
| COAG.DAF | 20 | 22.5 | −2.5 | 28 | 14 | 14 |
| INR.DAF | 21 | 20 | 1 | 28 | 26 | 2 |
| ACRF.DAF | 11 | 13 | −2 | 26 | 14 | 12 |
| ANYREN.DAF | 5.5 | 8.5 | −3 | 19 | 14 | 5 |
| RENSUP.DAF | 6.5 | 14.5 | −8 | 25 | 14 | 11 |
| ACHEP.DAF | 20.5 | 28 | −7.5 | 28 | 23 | 5 |
| ANYHEP.DAF | 18.5 | 28 | −9.5 | 28 | 19 | 9 |

Patients not treated with XIGRIS™ (2^{nd}, 3^{rd} and 4^{th} columns), the survival and organ dysfunction scored as days alive and free of organ dysfunction of patients who have (risk-risk) is compared to patients who carried one copy of either protein C 4732C or EPCR 4054 (risk-protective) and the protective-protective groups combined into a group called “other” by subtracting the survival and organ dysfunction scored as days alive and free of organ dysfunction of (risk-risk) (marked column A) from the survival and organ dysfunction scored as days alive and free of organ dysfunction of patients who carried one copy of either protein C 4732C or EPCR,4054 (risk-protective) and the protective-protective groups combined into a group called “other” (marked column B) shown in the fourth column (marked column A-B). It can be seen that in patients not treated with XIGRIS™, patients who carried at least one copy of protein C 4732C and one copy of EPCR 4054 T (risk-risk) do consistently worse as indicated by lower survival and fewer days alive and free of organ dysfunction (hence negative values in column A-B) than patients who carried one copy of either protein C 4732C or EPCR 4054 (risk-protective) and the protective-protective groups combined into a group called “other”.

Patients treated with XIGRIS™ (5^{th}, 6^{th} and 7^{th} columns), the survival and organ dysfunction scored as days alive and free of organ dysfunction of patients who have (risk-risk) is compared to patients who carried one copy of either protein C 4732C or EPCR 4054 (risk-protective) and the protective-protective groups combined into a group called “other” by subtracting the survival and organ dysfunction scored as days alive and free of organ dysfunction of (risk-risk) (marked column C) from the survival and organ dysfunction scored as days alive and free of organ dysfunction of patients who carried one copy of either protein C 4732C or EPCR 4054 (risk-protective) and the protective-protective groups combined into a group called “other” (marked column D) shown in the seventh column (marked column C-D). It can be seen that in patients who are treated with XIGRIS™, patients who have (risk-risk) do consistently better as shown by higher survival and more days alive and free of organ dysfunction) than patients who carried one copy of either protein C 4732C or EPCR 4054 (risk-protective) and the protective-protective groups combined into a group called “other”. This emphasizes the beneficial effects of XIGRIS™ in patients who carried at least one copy of protein C 4732C and one copy of EPCR 4054 T (risk-risk).

### Example 4D

### Effect of XIGRIS™ on protein C 2418

Similar to the results above for protein C 4732 and EPCR 4054 (alone and in combination), XIGRIS™ showed an absolute increase in survival prediction according to protein C 2418 AA genotype (risk) and was useful in predicting benefit from treatment with XIGRIS™. Even with a biased control sample, patients not treated with XIGRIS™ showed a 58% sepsis survival, whereas Sepsis survival in patients treated with XIGRIS™ was 64% for an absolute increase of 6% and a relative increase of 10%.

### Example 5

### Protein C T4732C in Cohort of Critically Ill Asian Patients

To reduce the potential for error due to population stratification analysis was also carried out on an Asian patient population. Of these 126 were successfully genotyped for protein C T4732C. Inclusion criteria, genotyping, clinical phenotype determinations, sample collection and statistical analysis were as described above.

Protein C T4732C was determined in a cohort of 126 ICU patients all of whom had SIRS. 33% of patients were TT homozygotes; 52% of patients were TC heterozygotes, and 15% of patients were CC homozygotes. The frequency of the T allele was 59% and the frequency of the C allele was 41% and these alleles were in Hardy Weinberg equilibrium in this population (Table 26). There were no differences in age, gender and APACHE II score, and distribution of medical vs. surgical admission status between protein C 4732 TT, CT and CC (Table 26).

TABLE 26
Baseline characteristics of critically ill Asian patients who had
SIRS who were genotyped for protein C 4732 T/C. There were
no differences in baseline characteristics (age, gender,
% surgical, APACHE II score) according to genotype.

| | Protein C | Protein C | Protein C |
|---|---|---|---|
| Variable | 4732 TT | 4732 TC | 4732 CC |
| N (%) | 41 (33%) | 66 (52%) | 19 (15%) |
| Age (mean ± SE) | 64 ± 3 | 62 ± 2 | 66 ± 4 |
| Gender (% Male) | 63 | 62 | 58 |
| APACHE II Score | 25 ± 1 | 23 ± 1 | 26 ± 2 |
| (mean ± SE) | | | |
| % Surgical | 34 | 15 | 21 |
| Diagnosis | | | |

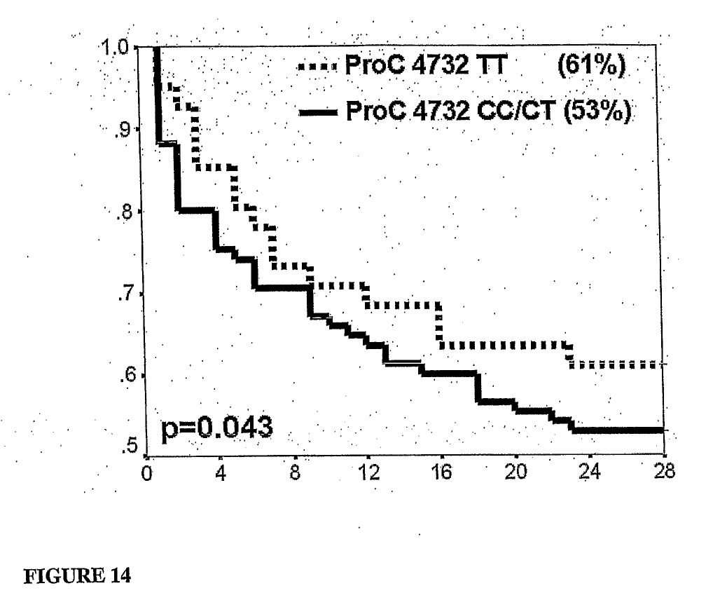

There was a significant association of protein C 4732 T/C genotype with 28-day survival in critically ill Asian patients with SIRS, Patients with the CC or CT genotype had a survival of 53% while patients with the TT genotype had a survival of 61% (Table 27, FIG. 14). This association was significant upon Cox proportional hazard regression analysis (p=0.043,Table 28).

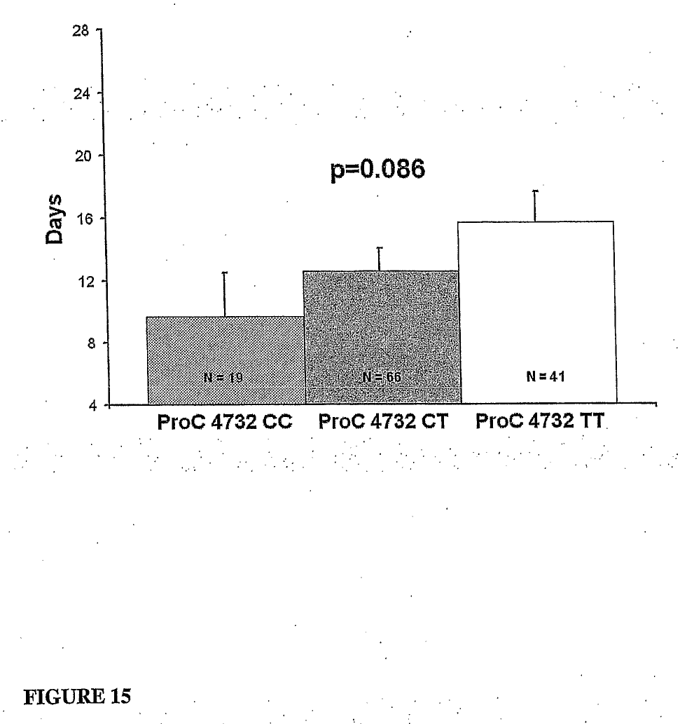

There was a trend toward association of protein C 4732 T/C genotype and days alive and free of steroid support (p=0.086) C(able 27, FIG. 15).

TABLE 27
Organ dysfunction of critically ill Asian patients who had SIRS
who were genotyped for protein C 4732 T/C.

| | Protein C | Protein C | Protein C | |
|---|---|---|---|---|
| Variable | 4732 TT | 4732 TC | 4732 CC | p Value |
| 28-Day Survival (%) | 61 | 53 | 53 | 0.043 |
| DAF Steroid Support | 16 ± 2 | 13 ± 1 | 10 ± 3 | 0.025 |
| (mean ± SE) | | | | |

TABLE 28
Cox proportional hazard regression analysis on 28-day survival with
covariates age, sex, APACHE II score, surgical diagnosis and
protein C 4732 T/C genotype in critically ill Asian patients SIRS.

| | | 95% | |
|---|---|---|---|
| Covariate | Hazard Ratio | Confidence Interval | p Value |
| APACHE II Score | 1.09 | 1.05-1.13 | <0.001 |
| Protein C 4732 CC or | 1.86 | 1.02-3.41 | 0.043 |
| CT Genotype | | | |
| Age | 1.02 | 1.00-1.04. | 0.058 |
| Surgical Diagnosis | 1.60 | 0.88-2.92 | 0.125 |
| Sex | 1.01 | 0.55-1.84 | 0.972 |

| | Protein C | Protein C | Protein C |
|---|---|---|---|
| Variable | 4732 TT | 4732 TC | 4732 CC |
| N (%) | 32 (32%) | 52 (52%) | 16 (16%) |
| Age (mean ± SE) | 67 ± 3 | 63 ± 2 | 68 ± 4 |
| Gender (% Male) | 62 | 63 | 62 |
| APACHE II Score | 25 ± 2 | 24 ± 1 | 28 ± 2 |
| (mean ± SE) | | | |
| % Surgical | 34 | 19 | 19 |
| Diagnosis | | | |

Sepsis Subgroup

We examined the protein C 4732 T/C polymorphism in a subgroup of 100 critically ill Asians all of whom had sepsis as defined in the methods. 34% of patients were TT homozygotes, 49% were TC heterozygotes and 18% were CC homozygotes. The frequency of the T allele was 58% and the frequency of the C allele was 42%, and these alleles were in Hardy-Weinberg equilibrium. There were no significant differences in age, gender, APACHE II score or distribution of medical vs. surgical admission status between protein C 4732 4732 TT, TCT or CC genotype groups.

Baseline characteristics of critically ill Asian patients who had sepsis who were genotyped for protein C 4732 T/C. There were no differences in baseline characteristics (age, gender, % surgical, APACHE II score) according to genotype.

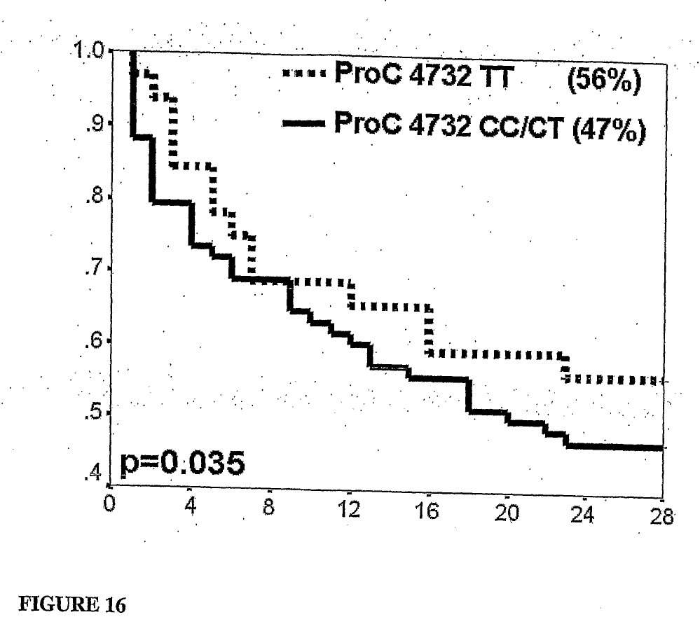

There was a significant association of protein C 4732 T/C genotype with 28-day survival in critically ill Asian patients with sepsis. Patients with the CC or CT genotype had a survival of 47% while patients with the TT genotype had a survival of 56% (Table 29, FIG. 16). This association was significant upon Cox proportional hazard regression analysis (p=0.035,Table 30).

TABLE 29
Organ dysfunction of critically ill Asian patients who had sepsis
who were genotyped for protein C 4732 T/C.

| | Protein C | Protein C | Protein C | p |
|---|---|---|---|---|
| Variable | 4732 TT | 4732 TC | 4732 CC | Value |
| 28-Day Survival (%) | 56 | 48 | 44 | 0.035 |
| DAF Steroid Support | 16 ± 2 | 11 ± 2 | 8 ± 3 | 0.020 |
| (mean ± SE) | | | | |
| DAF Coagulation | 18 ± 2 | 15 ± 2 | 12 ± 3 | 0.038 |
| (mean ± SE) | | | | |

TABLE 30
Cox proportional hazard regression analysis on 28-day survival
with covariates age, sex, APACHE II score, surgical diagnosis
and protein C 4732 T/C genotype in critically ill Asian patients
with sepsis.

| | | 95% | |
|---|---|---|---|
| Covariate | Hazard Ratio | Confidence Interval | p Value |
| APACHE II Score | 1.08 | 1.04-1.12 | <0.001 |
| Age | 1.02 | 1.00-1.05 | 0.019 |
| Protein C 4732 CC or | 2.00 | 1.05-3.81 | 0.035 |
| CT Genotype | | | |
| Surgical Diagnosis | 1.66 | 0.89-3.08 | 0.111 |
| Sex | 1.05 | 0.56-1.96 | 0.877 |

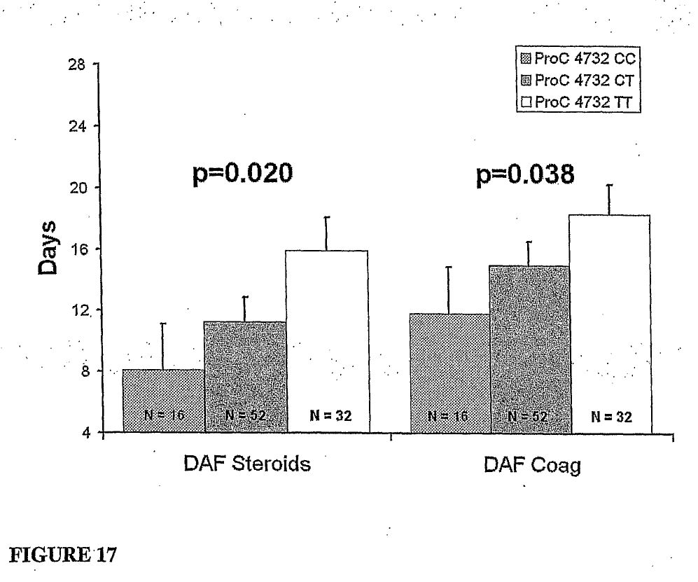

There was a significant association of protein C 4732 T/C genotype with days alive and free of steroid use (p=0.020) and between genotype and days alive and free of coagulation (p=0.038) in critically ill Asians with sepsis (Table 29, FIG. 17).

### SUMMARY

Clinical Implications

Subjects with sepsis, severe sepsis or SIRS may be genotyped to assess their protein C 2418, protein C 4732 and EPCR 4054 genotypes or the genotypes of polymorphic sites in linkage disequilibrium with protein C 2418, protein C 4732 and EPCR 4054. Subjects could then be classified by genotype into a risk category regarding their unique risk of death by genotype. Furthermore, the subject's genotype can be used to also determine how well they will respond to activated protein C (XIGRIS™) or other anti-inflammatory agents or anti-coagulant agents. It was found that there was a clear graded increase in absolute survival when XIGRIS™ was used as treatment according to the genotype of the subject for EPCR 4054, protein C 2418, protein C 4732, and the combined risk-risk genotype of protein C 4732/EPCR 4054. Thus, clinicians can now administer XIGRIS™ according to a subject-tailored risk assessment. Each subject's unique genotype of protein C and EPCR can be used to make a unique assessment of risk of death and to predict whether or not a subject is likely to benefit from XIGRIS™ treatment.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for obtaining a prognosis for a subject having, or at risk of developing, an inflammatory condition, the method comprising determining a genotype of said subject which includes one or more polymorphic sites in the subject's protein C sequence; EPCR sequence or a combination thereof, wherein said genotype is indicative of an ability of the subject to recover from the inflammatory condition.

2. The method of claim 1, wherein the polymorphic site is
(a) position 4732 Of SEQ ID NO: 1; or position 4054 of SEQ ID NO:2; or a polymorphic site in linkage disequilibrium thereto; or
(b) a combination of Protein C and EPCR sequences, wherein said polymorphic sites are at two or more of positions selected from 4732 of SEQ ID NO:1: 4054 of SEQ ID NO:2, 2418 of SEQ ID NO:1; and a polymorphic site in linkage disequilibrium thereto.

3. (canceled)

4. The method of claim 2, wherein
(a) the polymorphic site in linkage disequilibrium with position 4732 may be selected from positions 4813, 6379, 6762, 7779, 8058, 8915 and 12228 of SEQ ID NO:1;
(b) the polymorphic site in linkage disequilibrium with position 4054 may be selected from positions 2973, 3063, 3402. 4946, 5515 and 6196 of SEQ ID NO:2;
(c) the polymorphic site in linkage disequilibrium with position 2418 may be selected from positions 1386, 2583 and 3920 in SEQ ID NO:1;
(d) the polymorphic site in linkage disequilibrium with position 4732 may be selected from a combination of two polymorphic sites, which sites occur at any of the following combinations of positions in SEQ ID NO:1:
9198 and 5867;
9198 and 4800;
3220 and 5867; and
3220 and 4800;
and/or
(e) the polymorphic site in linkage disequilibrium with position 2418 may be selected from a combination of two polymorphic sites which sites occur at any of the following combinations of positions in SEQ ID NO: 1:
5867 and 2405;
5867 and 4919;
5867 and 4956;
5867 and 6187;
5867 and 12109;
4800 and 2405;
4800 and 4919;
4800 and 4956;
4800 and 6187; and
4800 and 12109.

5-9. (canceled)

10. The method of claim 1, further comprising obtaining protein C sequence information or EPCR sequence information for the subject.

11. The method of claim 1, wherein the genotype is determined using a nucleic acid sample from the subject.

12. The method of claim 11, further comprising obtaining the nucleic acid sample from the subject.

13. The method of claim 1, wherein said genotype is determined using one or more of the following techniques:
(a) restriction fragment length analysis;
(b) sequencing;
(c) hybridization;
(d) oligonucleotide ligation assay;
(e) ligation rolling circle amplification;
(f) 5′ nuclease assay;
(g) polymerase proofreading methods;
(h) allele specific PCR; and
(i) reading sequence data.

14. The method of claim 1, wherein
(a) the genotype of the subject is indicative of a decreased ability to recover from the inflammatory condition, or
(b) the subject is critically ill and the genotype is indicative of a prognosis of severe cardiovascular or respiratory dysfunction.

15. (canceled)

16. The method of claim 14, wherein the genotype comprises
(a) at least one of the following single polymorphic nucleotides or combinations of polymorphic nucleotides at the indicated positions of SEQ ID NO: 1:
4732 C;
4813 A;
6379 G:
6762 A;
7779 C;
8058 T;
8915 T;
12228 T;
9198 C and 5867 A;
9198 C and 4800 G;
3220 A and 5867 A; and
3220 A and 4800 G,
or
1386 T;
2418 A;
2583 A;
3920T:
5867 A and 2405 T;
5867 A and 4919 A;
5867 A and 4956 T;
5867 A and 6187 C;
5867 A and 12109 T;
4800 G and 2405 T;
4800 G and 4919 A;
4800 G and 4956 T;
4800 G and 6187 C; and
4800 G and 12109 T;
and
(b) at least one of the following EPCR polymorphic nucleotides at the indicated positions of SEQ ID NO: 2:
6196 G;
5515 T;
4946 T;
4054 T;
3402 G;
3063 G; and
2973 C.

17. (canceled)

18. The method of claim 16, wherein the genotype of the subject is indicative of an increased ability to recover from the inflammatory condition.

19. The method of claim 18, wherein the subject is critically ill and the genotype is indicative of a prognosis of mild cardiovascular or respiratory dysfunction.

20. The method of claim 14, wherein the genotype comprises
(a) at least one of the following genotypes or genotype combinations within SEQ ID NO: 1:
4732 T;
4813 G;
6379 A;
6762 G;
7779-;
8058 C;
8915 G;
12228 C;
9198 A and 5867 G;
9198 A and 4800 C;
3220 G and 5867 G; and
3220 G and 4800 C,
or
1386 C;
2418 G;
2583 T;
3920 C;
5867 G and 2405 C;
5867 G and 4919 G;
5867 G and 4956 C;
5867 G and 6187 T;
5867 G and 12109 C;
4800 C and 2405 C;
4800 C and 4919 G;
4800 C and 4956 C;
4800 C and 6187 T; and
4800 C and 12109 C;
and
(b) at least one of the following genotypes within SEQ ID NO: 2:
6196 C;
5515 C;
4946 C;
4054 C;
3402 C;
3063 A; and
2973 T.

21. (canceled)

22. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystis carinii, pneumonia, Leishmaniasis, hemolytic uremic syndrome/thrombotic thrombocytopenic purpura. Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

23. The method of claim 22, wherein the inflammatory condition is SIRS.

24-31. (canceled)

32. A method for selecting a group of subjects for determining the efficacy of a candidate drug known or suspected of being useful for the treatment of an inflammatory condition, the method comprising determining a genotype at one or more polymorphic sites in the protein C sequence or EPCR sequence for each subject, wherein said genotype is indicative of the subject's ability to recover from the inflammatory condition and sorting subjects based on their genotype.

33. The method of claim 32 further comprising, administering the candidate drug to the subjects or a subset of subjects and determining each subject's ability to recover from the inflammatory condition.

34. The method of claim 33, further comprising comparing subject response to the candidate drug based on genotype of the subject.

35. (canceled)

36. A method of treating an inflammatory condition in a subject in need thereof, the method comprising:
(a) selecting a subject having a risk genotype in their protein C sequence or EPCR sequence; and
(b) administering to said subject an anti-inflammatory agent or an anti-coagulant agent.

37-43. (canceled)

44. The method of claim 36, further comprising determining the subject's APACHE II score as an assessment of subject risk.

45. The method of claim 36, further comprising determining the number of organ system failures for the subject as an assessment of subject risk.

46. The method of claim 44, wherein, an APACHE II score ≧25 is indicative of increased risk.

47. The method of claim 45, wherein two or more organ system failures are indicative of increased subject risk.

48. The method of claim 36, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystis carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

49. The method of claim 48, wherein the inflammatory condition is systemic inflammatory response syndrome.

50. The method claim 36, wherein the risk genotype is located at a polymorphic site at one or more of the following positions: 4732 of SEQ ID NO:1; 4054 of SEQ ID NO:2; 2418 of SEQ ID NO:1; or a polymorphic site or combination of sites in linkage disequilibrium thereto.

51-52. (canceled)

53. The method of claim 50, wherein the polymorphic site in linkage disequilibrium with position 4732 is at
(a) position 4813, 6379, 6762, 7779, 8058, 8915 or 12228 of SEQ ID NO: 1;
(b) position 2973, 3063, 3402, 4946, 5515 or 6196 of SEQ ID NO:2; or
(c) position 1386, 2583 or 3920 in SEQ ID NO:1.

54-55. (canceled)

56. The method of claim 50, wherein the risk genotype is selected from the group of positions in SEQ ID NO: 1 consisting of:
9198 and 5867;
9198 and 4800;
3220 and 5867;
3220 and 4800;
5867 and 2405;
5867 and 4919;
5867 and 4956;
5867 and 6187;
5867 and 12109;
4800 and 2405;
4800 and 4919;
4800 and 4956;
4800 and 6187; and
4800 and 12109.

57. (canceled)

58. The method of claim 36, wherein the risk genotype is selected from:
(a) the following genotypes in SEQ ID NO: 1:
4732 C;
4813 A;
6379 G;
6762 A;
7779 C;
8058 T;
8915 T;
12228 T;
9198 C and 5867 A;
9198 C and 4800 G:
3220 A and 5867 A;
3220 A and 4800 G
1386 T:
2418 A;
2583 A;
3920 T;
5867 A and 2405 T;
5867 A and 4919 A;
5867 A and 4956 T;
5867 A and 6187 C;
5867 A and 12109 T;
4800 G and 2405 T;
4800 G and 4919 A;
4800 G and 4956 T;
4800 G and 6187 C; and
4800 G and 12109T;
and
(b) the following genotypes in SEQ ID NO: 2:
6196 G;
5515 T;
4946 T;
4054 T;
3402 G;
3063 G; and
2973 C.

59. (canceled)

60. The method of claim 58, wherein the genotype of the subject is indicative of an increased risk of poor outcome from an inflammatory condition.

61. The method of claim 60, wherein the subject having an increased risk of poor outcome from an inflammatory condition is preferentially selected for administration of the anti-inflammatory agent or the anti-coagulant agent.

62. The method of claim 36, wherein the genotype for a decreased risk is selected from:
(a) the following genotypes in SEQ ID NO: 1:
4732 T;
4813 G;
6379 A;
6762 G;
7779 -;
8058 C;
8915 G;
12228 C;
9198 A and 5867 G;
9198 A and 4800 C;
3220 G and 5867 G;
3220 G and 4800 C;
1386 C;
2418 G;
2583 T;
3920 C;
5867 G and 2405 C:
5867 G and 4919 G;
5867 G and 4956 C;
5867 G and 6187 T;
5867 G and 12109 C;
4800 C and 2405 C;
4800 C and 4919 G;
4800 C and 4956 C;
4800 C and 6187 T; and
4800 C and 12109 C;
and
(b) the following genotypes in SEQ ID NO: 2:
6196 C;
5515 C;
4946 C;
4054 C;
3402 C;
3063 A; and
2973 T.

63. (canceled)

64. The method of claim 62, wherein the genotype of the subject is indicative of a decreased risk of poor outcome from an inflammatory condition.

65. The method of claim of 64, wherein the subject having a decreased risk of poor outcome from an inflammatory condition is preferentially not selected for administration the anti-inflammatory agent or the anti-coagulant agent.

66. The method of claim 36, wherein the anti-inflammatory agent or the anti-coagulant agent is one or more of:
(a) activated protein C;
(b) a tissue factor pathway inhibitor;
(c) platelet activating factor hydrolase;
(d) PAF-AH enzyme analogues;
(e) antibody to tumor necrosis factor α alpha;
(f) soluble tumor necrosis factor receptor-immunoglobulin G1;
(g) procysteine;
(h) elastase inhibitor;
(i) a human recombinant interleukin 1 receptor antagonist; and
(j) an antibody specific for, an inhibitor of, or an antagonist of: endotoxin, tumour necrosis factor receptor, interleukin-6, high mobility group box, tissue plasminogen activator, bradykinin, CD-14 or interleukin-10.

67. The method of claim 66, wherein the anti-inflammatory agent or anti-coagulant agent is activated protein C.

68. The method of claim 36, wherein the anti-inflammatory anti-coagulant agent is drotecogin alfa activated.

69. An oligonucleotide of about 10 to about 400 nucleotides that hybridizes specifically to a sequence contained in a human target sequence consisting of:
(a) SEQ ID NO:1, a complementary sequence of the target sequence or RNA equivalent of the target sequence and wherein the oligonucleotide is operable in determining a polymorphism genotype at position 4732, 4813, 6379, 6762, 7779, 8058, 8915, 12228, 9198, 5867, 4800, 3220, 1386, 2418, 2583, 3920, 2405, 4919, 4956, 6187 or 12109 of SEQ ID NO:1; or
(b) SEQ ID NO:2, a complementary sequence of the target sequence or RNA equivalent of the target sequence and wherein the oligonucleotide is operable in determining a polymorphism genotype at position 6196, 5515, 4946, 4054, 3402, 3063 or 2973 of SEQ ID NO:2.

70-72. (canceled)

73. An oligonucleotide probe selected from the group consisting of:
(a) a probe that hybridizes under high stringency conditions to a nucleic acid molecule comprising SEQ ID NO:1 having a C at position 4732 but not to a nucleic acid molecule comprising SEQ ID NO:1 having a T at position 4732;
(b) a probe that hybridizes under high stringency conditions to a nucleic acid molecule comprising SEQ ID NO:1 having a T at position 4732 but not to a nucleic acid molecule comprising SEQ ID NO:1 having a C at position 4732;
(c) a probe that hybridizes under high stringency conditions to a nucleic acid molecule comprising SEQ ID NO:1 having a A at position 4813 but not to a nucleic acid molecule comprising SEQ ID NO:1 having a G at position 4813;
(d) a probe that hybridizes under high stringency conditions to a nucleic acid molecule comprising SEQ ID NO:1 having a G at position 4813 but not to a nucleic acid molecule comprising SEQ ID NO:1 having a A at position 4813;
(e) a probe that hybridizes under high stringency conditions to a nucleic acid molecule comprising SEQ ID NO:1 having a A at position 6379 but not to a nucleic acid molecule comprising SEQ ID NO:1 having a G at position 6379;
(f) a probe that hybridizes under high stringency conditions to a nucleic acid molecule comprising SEQ ID NO:1 having a G at position 6379 but not to a nucleic acid molecule comprising SEQ ID NO:1 having a A at position 6379;
(g) a probe that hybridizes under high stringency conditions to a nucleic acid molecule comprising SEQ ID NO:1 having a A at position 6762 but not to a nucleic acid molecule comprising SEQ ID NO:1 having a G at position 6762;
(h) a probe that hybridizes under high stringency conditions to a nucleic acid molecule comprising SEQ ID NO:1 having a G at position 6762 but not to a nucleic acid molecule comprising SEQ ID NO:1 having a A at position 6762;
(i) a probe that hybridizes under high stringency conditions to a nucleic acid molecule comprising SEQ ID NO:1 having a C at position 7779 but not to a nucleic acid molecule comprising SEQ ID NO:46 having a T at position 7779;
(j) a probe that hybridizes under high stringency conditions to a nucleic acid molecule comprising SEQ ID NO:46 having a T at position 7779 but not to a nucleic acid molecule comprising SEQ ID NO:I having a C at position 7779;
(k) a probe that hybridizes under high stringency conditions to a nucleic acid molecule comprising SEQ ID NO:1 having a T at position 8058 but not to a nucleic acid molecule comprising SEQ ID NO:1 having a C at position 8058;
(l) a probe that hybridizes under high stringency conditions to a nucleic acid molecule comprising SEQ ID NO:1 having a C at position 8058 but not to a nucleic acid molecule comprising SEQ ID NO:1 having a T at position 8058;
(m) a probe that hybridizes under high stringency conditions to a nucleic acid molecule comprising SEQ ID NO:1 having a T at position 8915 but not to a nucleic acid molecule comprising SEQ ID NO:1 having a G at position 8915;
(n) a probe that hybridizes under high stringency conditions to a nucleic acid molecule comprising SEQ ID NO:1 having a G at position 8915 but not to a nucleic acid molecule comprising SEQ ID NO:1 having a T at position 8915;
(o) a probe that hybridizes under high stringency conditions to a nucleic acid molecule comprising SEQ ID NO:1 having a T at position 12228 but not to a nucleic acid molecule comprising SEQ ID NO:1 having a C at position 12228;
(p) a probe that hybridizes under high stringency conditions to a nucleic acid molecule comprising SEQ ID NO:1 having a C at position 12228 but not to a nucleic acid molecule comprising SEQ ID NO:1 having a in at position 12228;
(q) a probe that hybridizes under high stringency conditions to a nucleic acid molecule comprising SEQ ID NO:1 having a A at position 5867 but not to a nucleic acid molecule comprising SEQ ID NO:1 having a G at position 5867;
(r) a probe that hybridizes under high stringency conditions to a nucleic acid molecule comprising SEQ ID NO:1 having a G at position 5867 but not to a nucleic acid molecule comprising SEQ ID NO:1 having a A at position 5867;
(s) a probe that hybridizes under high stringency conditions to a nucleic acid molecule comprising SEQ ID NO:1 having a C at position 9198 but not to a nucleic acid molecule comprising SEQ ID NO:1 having a A at position 9198;
(t) a probe that hybridizes under high stringency conditions to a nucleic acid molecule comprising SEQ ID NO:1 having a A at position 9198 but not to a nucleic acid molecule comprising SEQ ID NO:1 having a C at position 9198;
(u) a probe that hybridizes under high stringency conditions to a nucleic acid molecule comprising SEQ ID NO:1 having a G at position 4800 but not to a nucleic acid molecule comprising SEQ ID NO:1 having a C at position 4800;
(v) a probe that hybridizes under high stringency conditions to a nucleic acid molecule comprising SEQ ID NO:1 having a C at position 4800 but not to a nucleic acid molecule comprising SEQ ID NO:1 having a G at position 4800;
(w) a probe that hybridizes under high stringency conditions to a nucleic acid molecule comprising SEQ ID NO:1 having a A at position 3220 but not to a nucleic acid molecule comprising SEQ ID NO:1 having a G at position 3220;
(x) a probe that hybridizes under high stringency conditions to a nucleic acid molecule comprising SEQ ID NO:1 having a G at position 3220 but not to a nucleic acid molecule comprising SEQ ID NO:1 having a A at position 3220;
(y) a probe that hybridizes under high stringency conditions to a nucleic acid molecule comprising SEQ ID NO:1 having a A at position 2418 but not to a nucleic acid molecule comprising SEQ ID NO:1 having a G at position 2418;
(z) a probe that hybridizes under high stringency conditions to a nucleic acid molecule comprising SEQ ID NO:1 having a G at position 2418 but not to a nucleic acid molecule comprising SEQ ID NO:1 having a A at position 2418;
(aa) a probe that hybridizes under high stringency conditions to a nucleic acid molecule comprising SEQ ID NO:2 having a C at position 2973 but not to a nucleic acid molecule comprising SEQ ID NO:2 having a T at position 2973;
(bb) a probe that hybridizes under high stringency conditions to a nucleic acid molecule comprising SEQ ID NO:2 having a T at position 2973 but not to a nucleic acid molecule comprising SEQ ID NO:2 having a C at position 2973;
(cc) a probe that hybridizes under high stringency conditions to a nucleic acid molecule comprising SEQ ID NO:2 having a G at position 3063 but not to a nucleic acid molecule comprising SEQ ID NO:2 having a A at position 3063;
(dd) a probe that hybridizes under high stringency conditions to a nucleic acid molecule comprising SEQ ID NO:2 having a A at position 3063 but not to a nucleic acid molecule comprising SEQ ID NO:2 having a G at position 3063;
(cc) a probe that hybridizes under high stringency conditions to a nucleic acid molecule comprising SEQ ID NO:2 having a G at position 3402 but not to a nucleic acid molecule comprising SEQ ID NO:2 having a C at position 3402;
(ff) a probe that hybridizes under high stringency conditions to a nucleic acid molecule comprising SEQ ID NO:2 having a C at position 3402 but not to a nucleic acid molecule comprising SEQ ID NO:2 having a G at position 3402;
(gg) a probe that hybridizes under high stringency conditions to a nucleic acid molecule comprising SEQ ID NO:2 having a T at position 4054 but not to a nucleic acid molecule comprising SEQ ID NO:2 having a C at position 4054;
(hh) a probe that hybridizes under high stringency conditions to a nucleic acid molecule comprising SEQ ID NO:2 having a C at position 4054 but not to a nucleic acid molecule comprising SEQ ID NO:2 having a T at position 4054;
(ii) a probe that hybridizes under high stringency conditions to a nucleic acid molecule comprising SEQ ID NO:2 having a T at position 4946 but not to a nucleic acid molecule comprising SEQ ID NO:2 having a C at position 4946;
(jj) a probe that hybridizes under high stringency conditions to a nucleic acid molecule comprising SEQ ID NO:2 having a C at position 4946 but not to a nucleic acid molecule comprising SEQ ID NO:2 having a T at position 4946;
(kk) a probe that hybridizes under high stringency conditions to a nucleic acid molecule comprising SEQ ID NO:2 having a T at position 5515 but not to a nucleic acid molecule comprising SEQ ID NO:2 having a C at position 5515;
(ll) a probe that hybridizes under high stringency conditions to a nucleic acid molecule comprising SEQ ID NO:2 having a C at position 5515 but not to a nucleic acid molecule comprising SEQ ID NO:2 having a T at position 5515;
(mm) a probe that hybridizes under high stringency conditions to a nucleic acid molecule comprising SEQ ID NO:2 having a G at position 6196 but not to a nucleic acid molecule comprising SEQ ID NO:2 having a C at position 6196; and
(nn) a probe that hybridizes under high stringency conditions to a nucleic acid molecule comprising SEQ ID NO:2 having a C at position 6196 but not to a nucleic acid molecule comprising SEQ ID NO:2 having a G at position 6196.

74. (canceled)

75. An array of nucleic acid molecules attached to a solid support, the array comprising one or more oligonucleotides selected from the group consisting of:
(a) an oligonucleotide that will hybridize to a nucleic acid molecule comprising SEQ ID NO:1 having a C at position 4732 but not to a nucleic acid molecule comprising SEQ ID NO:1 having a T at position 4732;
(b) an oligonucleotide that will hybridize to a nucleic acid molecule comprising SEQ ID NO:1 having a T at position 4732 but not to a nucleic acid molecule comprising SEQ ID NO:1 having a C at position 4732;
(c) an oligonucleotide that will hybridize to a nucleic acid molecule comprising SEQ ID NO:1 having a A at position 4813 but not to a nucleic acid molecule comprising SEQ ID NO:1 having a G at position 4813;
(d) an oligonucleotide that will hybridize to a nucleic acid molecule comprising SEQ ID NO:1 having a G at position 4813 but not to a nucleic acid molecule comprising SEQ ID NO:1 having a A at position 4813;
(e) an oligonucleotide that will hybridize to a nucleic acid molecule comprising SEQ ID NO:1 having a A at position 6379 but not to a nucleic acid molecule comprising SEQ ID NO:1 having a G at position 6379;
(f) an oligonucleotide that will hybridize to a nucleic acid molecule comprising SEQ ID NO:1 having a G at position 6379 but not to a nucleic acid molecule comprising SEQ ID NO:1 having a A at position 6379;
(g) an oligonucleotide that will hybridize to a nucleic acid molecule comprising SEQ ID NO:1 having a A at position 6762 but not to a nucleic acid molecule comprising SEQ ID NO:1 having a G at position 6762;
(h) an oligonucleotide that will hybridize to a nucleic acid molecule comprising SEQ ID NO:1 having a G at position 6762 but not to a nucleic acid molecule comprising SEQ ID NO:1 having a A at position 6762;
(i) an oligonucleotide that will hybridize to a nucleic acid molecule comprising SEQ ID NO:1 having a C at position 7779 but not to a nucleic acid molecule comprising SEQ ID NO:46 having a T at position 7779;
(j) an oligonucleotide that will hybridize to a nucleic acid molecule comprising SEQ ID NO:46 having a T at position 7779 but not to a nucleic acid molecule comprising, SEQ ID NO:1 having a C at position 7779;
(k) an oligonucleotide that will hybridize to a nucleic acid molecule comprising SEQ ID NO:1 having a T at position 8058 but not to a nucleic acid molecule comprising SEQ ID NO:1 having a C at position 8058;
(l) an oligonucleotide that will hybridize to a nucleic acid molecule comprising SEQ ID NO:1 having a C at position 8058 but not to a nucleic acid molecule comprising SEQ ID NO:1 having a T at position 8058;
(m) an oligonucleotide that will hybridize to a nucleic acid molecule comprising SEQ ID NO:1 having a T at position 8915 but not to a nucleic acid molecule comprising SEQ ID NO:1 having a G at position 8915;
(n) an oligonucleotide that will hybridize to a nucleic acid molecule comprising SEQ ID NO:1 having a G at position 8915 but not to a nucleic acid molecule comprising SEQ ID NO:1 having a T at position 8915;
(o) an oligonucleotide that will hybridize to a nucleic acid molecule comprising SEQ ID NO:1 having a T at position 12228 but not to a nucleic acid molecule comprising SEQ ID NO:1 having a C at position 12228;
(p) an oligonucleotide that will hybridize to a nucleic acid molecule comprising SEQ ID NO:1 having a C at position 12228 but not to a nucleic acid molecule comprising SEQ ID NO:1 having a T at position 12228;
(q) an oligonucleotide that will hybridize to a nucleic acid molecule comprising SEQ ID NO:1 having a A at position 5867 but not to a nucleic acid molecule comprising SEQ ID NO:1 having a G at position 5867;
(r) an oligonucleotide that will hybridize to a nucleic acid molecule comprising SEQ ID NO:1 having a G at position 5867 but not to a nucleic acid molecule comprising SEQ ID NO: 1 having a A at position 5867;
(s) an oligonucleotide that will hybridize to a nucleic acid molecule comprising SEQ ID NO:1 having a C at position 9198 but not to a nucleic acid molecule comprising SEQ ID NO:1 having a A at position 9198;
(t) an oligonucleotide that will hybridize to a nucleic acid molecule comprising SEQ ID NO:1 having a A at position 9198 but not to a nucleic acid molecule comprising SEQ ID NO:1 having a C at position 9198;
(u) an oligonucleotide that will hybridize to a nucleic acid molecule comprising SEQ ID NO:1 having a G at position 4800 but not to a nucleic acid molecule comprising SEQ ID NO:1 having a C at position 4800;
(v) an oligonucleotide that will hybridize to a nucleic acid molecule comprising SEQ ID NO:1 having a C at position 4800 but not to a nucleic acid molecule comprising SEQ ID NO:1 having a G at position 4800;
(w) an oligonucleotide that will hybridize to a nucleic acid molecule comprising SEQ ID NO:1 having a A at position 3220 but not to a nucleic acid molecule comprising SEQ ID NO:1 having a G at position 3220;
(x) an oligonucleotide that will hybridize to a nucleic acid molecule comprising SEQ ID NO:1 having a G at position 3220 but not to a nucleic acid molecule comprising SEQ ID NO:1 having a A at position 3220;
(y) an oligonucleotide that will hybridize to a nucleic acid molecule comprising SEQ ID NO:1 having a A at position 2418 but not to a nucleic acid molecule comprising SEQ ID NO:1 having a G at position 2418;
(z) an oligonucleotide that will hybridize to a nucleic acid molecule comprising SEQ ID NO:1 having a G at position 2418 but not to a nucleic acid molecule comprising SEQ ID NO:1 having a A at position 2418;
(aa) an oligonucleotide that will hybridize to a nucleic acid molecule comprising SEQ ID NO:2 having a C at position 2973 but not to a nucleic acid molecule comprising SEQ ID NO:2 having a T at position 2973;
(bb) an oligonucleotide that will hybridize to a nucleic acid molecule comprising SEQ ID NO:2 having a T at position 2973 but not to a nucleic acid molecule comprising SEQ ID NO:2 having a C at position 2973;
(cc) an oligonucleotide that will hybridize to a nucleic acid molecule comprising SEQ ID NO:2 having a G at position 3063 but not to a nucleic acid molecule comprising SEQ ID NO:2 having a A at position 3063;
(dd) an oligonucleotide that will hybridize to a nucleic acid molecule comprising SEQ ID NO:2 having a A at position 3063 but not to a nucleic acid molecule comprising SEQ ID NO:2 having a G at position 3063;
(ee) an oligonucleotide that will hybridize to a nucleic acid molecule comprising SEQ ID NO:2 having a G at position 3402 but not to a nucleic acid molecule comprising SEQ ID NO:2 having a C at position 3402;
(ff) an oligonucleotide that will hybridize to a nucleic acid molecule comprising SEQ ID NO:2 having a C at position 3402 but not to a nucleic acid molecule comprising SEQ ID NO:2 having a G at position 3402;
(gg) an oligonucleotide that will hybridize to a nucleic acid molecule comprising SEQ ID NO:2 having a T at position 4054 but not to a nucleic acid molecule comprising SEQ ID NO:2 having a C at position 4054;
(hh) an oligonucleotide that will hybridize to a nucleic acid molecule comprising SEQ ID NO:2 having a C at position 4054 but not to a nucleic acid molecule comprising SEQ ID NO:2 having a T at position 4054;
(ii) an oligonucleotide that will hybridize to a nucleic acid molecule comprising SEQ ID NO:2 having a T at position 4946 but not to a nucleic acid molecule comprising SEQ ID NO:2 having a C at position 4946;
(jj) an oligonucleotide that will hybridize to a nucleic acid molecule comprising SEQ ID NO:2 having a C at position 4946 but not to a nucleic acid molecule comprising SEQ ID NO:2 having a T at position 4946;
(kk) an oligonucleotide that will hybridize to a nucleic acid molecule comprising SEQ ID NO:2 having a T at position 5515 but not to a nucleic acid molecule comprising SEQ ID NO:2 having a C at position 5515;
(ll) an oligonucleotide that will hybridize to a nucleic acid molecule comprising SEQ ID NO:2 having a C at position 5515 but not to a nucleic acid molecule comprising SEQ ID NO:2 having a T at position 5515;
(mm) an oligonucleotide that will hybridize to a nucleic acid molecule comprising SEQ ID NO:2 having a G at position 6196 but not to a nucleic acid molecule comprising SEQ ID NO:2 having a C at position 6196;
(nn) an oligonucleotide that will hybridize to a nucleic acid molecule comprising SEQ ID NO:2 having a C at position 6196 but not to a nucleic acid molecule comprising SEQ ID NO:2 having a G at position 6196;
(oo) an oligonucleotide that will hybridize to a nucleic acid molecule comprising SEQ ID NO:3 having a G at position 201 but not to a nucleic acid molecule represented by the same SEQ ID NO having a A at position 201;
(pp) an oligonucleotide that will hybridize to a nucleic acid molecule comprising SEQ ID NO:3 having a A at position 201 but not to a nucleic acid molecule represented by the same SEQ ID NO having a G at position 201;
(qq) an oligonucleotide that will hybridize to a nucleic acid molecule comprising SEQ ID NO:4, SEQ ID NO:10, SEQ ID NO:11, SEQ ID NO:14, SEQ ID NO:15, SEQ ID NO:18, SEQ ID NO:19, or SEQ ID NO:20 having a T at position 201 but not to a nucleic acid molecule represented by the same SEQ ID NO having a C at position 201;
(rr) an oligonucleotide that will hybridize to a nucleic acid molecule comprising SEQ ID NO:4, SEQ ID NO:10, SEQ ID NO:11, SEQ ID NO:14, SEQ ID NO:15, SEQ ID NO:18, SEQ ID NO:19, or SEQ ID NO:20 having a C at position 201 but not to a nucleic acid molecule represented by the same SEQ ID NO having a T at position 201;
(ss) an oligonucleotide that will hybridize to a nucleic acid molecule comprising SEQ ID NO:5, SEQ ID NO:17, or SEQ ID NO:21 having a C at position 201 but not to a nucleic acid molecule represented by the same SEQ ID NO having a G at position 201;
(tt) an oligonucleotide that will hybridize to a nucleic acid molecule comprising SEQ ID NO:5, SEQ ID NO:17, or SEQ ID NO:21 having a G at position 201 but not to a nucleic acid molecule represented by the same SEQ ID NO having a C at position 201;
(uu) an oligonucleotide that will hybridize to a nucleic acid molecule comprising SEQ ID NO:12 having, a G at position 201 but not to a nucleic acid molecule comprising SEQ ID NO:12 having a T at position 201;
(vv) an oligonucleotide that will hybridize to a nucleic acid molecule comprising SEQ ID NO:12 having a T at position 201 but not to a nucleic acid molecule comprising SEQ ID NO:12 having a G at position 201;
(ww) an oligonucleotide that will hybridize to a nucleic acid molecule comprising SEQ ID NO:12 having a T at position 201 but not to a nucleic acid molecule comprising SEQ ID NO:12 having a G at position 201;
(xx) an oligonucleotide that will hybridize to a nucleic acid molecule comprising SEQ ID NO:13 having a A at position 201 but not to a nucleic acid molecule comprising SEQ ID NO:13 having a C at position 201;
(yy) an oligonucleotide that will hybridize to a nucleic acid molecule comprising SEQ ID NO:22 SEQ ID NO:23, or SEQ ID NO:41 having, a C at position 15 but not to a nucleic acid molecule represented by the same SEQ ID NO having a G at position 15;
(zz) an oligonucleotide that will hybridize to a nucleic acid molecule comprising SEQ ID NO:22, SE ID NO:23, or SEQ ID NO:41 having a G at position 15 but not to a nucleic acid molecule represented by the same SEQ ID NO having a C at position 15;
(aaa) an oligonucleotide that will hybridize to a nucleic acid molecule comprising SEQ ID NO:24, SEQ ID NO:27, SEQ ID NO:28, SEQ ID NO:29, SEQ ID NO:31, SEQ ID NO:35, SEQ ID NO:36, SEQ ID NO:39, or SEQ ID NO:44 having a C at position 15 but not to a nucleic acid molecule represented by the same SEQ ID NO having a G at position 15;
(bbb) an oligonucleotide that will hybridize to a nucleic acid molecule comprising SEQ ID NO:24, SEQ ID NO:27, SEQ ID NO:28, SEQ ID NO:29, SEQ ID NO:3 1, SEQ ID NO:35, SEQ ID NO:36, SEQ ID NO:39, or SEQ ID NO:44 having a G at position 15 but not to a nucleic acid molecule represented by the same SEQ ID NO having a C at position 15;
(ccc) an oligonucleotide that will hybridize to a nucleic acid molecule comprising SEQ ID NO:25 or SEQ ID NO:49 having a A at position 15 but not to a nucleic acid molecule represented by the same SEQ ID NO having a T at position 15;
(ddd) an oligonucleotide that will hybridize to a nucleic acid molecule comprising SEQ ID NO:25 or SEQ ID NO:49 having a T at position 15 but not to a nucleic acid molecule represented by the same SEQ ID NO having a A at position 15;
(eee) an oligonucleotide that will hybridize to a nucleic acid molecule comprising SEQ ID NO:26, SEQ ID NO:33, SEQ ID NO:34, SEQ ID NO:38, or SEQ ID NO:40 having a A at position 15 but not to a nucleic acid molecule represented by the same SEQ ID NO having a G at position 15;
(fff) an oligonucleotide that will hybridize to a nucleic acid molecule comprising SEQ ID NO:26, SEQ ID NO:33, SEQ ID NO:34, SEQ ID NO:38, or SEQ ID NO:40 having a G at position 15 but not to a nucleic acid molecule represented by the same SEQ ID NO having a A at position 15;
(ggg) an oligonucleotide that will hybridize to a nucleic acid molecule comprising SEQ ID NO:30, SEQ ID NO:32, SEQ ID NO:37, SEQ ID NO:42, or SEQ ID NO:43 having a C at position 15 but not to a nucleic acid molecule represented by the same SEQ ID NO having a A at position 15; and
(hhh) an oligonucleotide that will hybridize to a nucleic acid molecule comprising SEQ ID NO:30, SEQ ID NO:32, SEQ ID NO:37, SEQ ID NO:42, or SEQ ID NO:43 having a A at position 15 but not to a nucleic acid molecule represented by the same SEQ ID NO having a C at position 15.

76-78. (canceled)

79. An array of nucleic acid molecules attached to a solid support, the array comprising an oligonucleotide selected from one of more of the following:
(a) an oligonucleotide that will hybridize to a nucleic acid molecule consisting of SEQ ID NO:1, wherein the nucleotide at position 4732 is C, under conditions in which the oligonucleotide will not substantially hybridize to a nucleic acid molecule consisting of SEQ ID NO:1 wherein the nucleotide at position 4732 is T;
(b) an oligonucleotide that will hybridize to a nucleic acid molecule consisting of SEQ ID NO:1, wherein the nucleotide at position 4732 is T, under conditions in which the oligonucleotide will not substantially hybridize to a nucleic acid molecule consisting of SEQ ID NO:1 wherein the nucleotide at position 4732 is C;
(c) an oligonucleotide that will hybridize to a nucleic acid molecule consisting of SEQ ID NO:2, wherein the nucleotide at position 4054 is T, under conditions in which the oligonucleotide will not substantially hybridize to a nucleic acid molecule consisting of SEQ ID NO:2 wherein the nucleotide at position 4054 is C; and
(d) an oligonucleotide that will hybridize to a nucleic acid molecule consisting of SEQ ID NO:1, wherein the nucleotide at position 4054 is C, under conditions in which the oligonucleotide will not substantially hybridize to a nucleic acid molecule consisting of SEQ ID NO:1 wherein the nucleotide at position 4054 is T.

80-82. (canceled)

83. The oligonucleotide of claim 69, further comprising one or more of the following:
(i) a detectable label;
(ii) a quencher;
(iii) a mobility modifier; or
(iv) a contiguous non-target sequence situated
(1) 5′ or 3′ to the target sequence, or
(2) 5′ and 3′ to the target sequence.

84. A computer readable medium comprising a plurality o f digitally encoded genotype correlations selected from the Protein C and EPCR genotype correlations in TABLE 1E, wherein each correlation of the plurality has a value representing an ability to recover from an inflammatory condition and an indication of responsiveness to treatment of an inflammatory condition with an anti-inflammatory agent or an anti-coagulant agent.

85. The oligonucleotide of claim 73, further comprising one or more of the following:
(i) a detectable label;
(ii) a quencher;
(iii) a mobility modifier;
(iv) a contiguous non-target sequence situated
(1) 5′ or 3′ to the target sequence, or
(2) 5′ and 3′ to the target sequence.

86. The array of claim 75, wherein the oligonucleotide further comprising one or more of the following:
i) a detectable label;
(ii) a quencher;
(iii) a mobility modifier;
(iv) a contiguous non-target sequence situated
(1) 5′ or 3′ to the target sequence, or
(2) 5′ and 3′ to the target sequence.

87. The array of claim 79, wherein the oligonucleotide further comprising one or more of the following:
i) a detectable label;
(ii) a quencher;
(iii) a mobility modifier;
(iv) a contiguous non-target sequence situated
(1) 5′ or 3′ to the target sequence, or
(2) 5′ and 3′ to the target sequence.
